(19) United States
(12) Patent Application Publication
(10) Pub. No.: US 2026/0327604 A1
(43) Pub. Date: Oct. 22, 2026

(54) TAGMENTATION AND LOOPED LIGATION LIBRARY PREPARATION

(71) Applicant: Canal Biosciences, Inc., Lowell, MA (US)

(72) Inventors: Yu ZHENG, Lowell, MA (US); Matthew HIMS, Lowell, MA (US)

(21) Appl. No.: 19/643,321

(22) Filed: Apr. 9, 2026

Related U.S. Application Data
(60) Provisional application No. 63/785,962, filed on Apr. 9, 2025.

(51) Int. Cl. C12N 15/10; C12N 9/00; C12N 9/12; C12N 9/16; C12N 9/24; C12N 9/88

(52) U.S. Cl. CPC C12N 15/1065; C12N 9/1241; C12N 9/16; C12N 9/2497; C12N 9/88; C12N 9/93; C12Y 301/21002; C12Y 302/02027; C12Y 402/99018; C12Y 605/01003

(57) ABSTRACT

Provided herein are compositions, kits, systems, and methods for tagmenting duplex nucleic acid molecules with at least partially double-stranded transposons to generate duplex fragments with loop-like adapters at each end. In some embodiments, the at least partially double stranded transposons herein comprise first and second adapter sequences (e.g., that are separated by a cleavable region), or have a first adapter (e.g., with no cleavable region). In certain embodiments, at least one of the loop like adapters are cleaved at a cleavable region to generate a two-part adapter at one end, or at each end, of the duplex fragment. In certain embodiments, such two-part adapter duplex fragments are subjected to amplification and sequenced to generate sequence reads.

The present application claims priority to U.S. Provisional application Ser. No. 63/785,962, filed on Apr. 9, 2025, which is herein incorporated by reference in its entirety.

### SEQUENCE LISTING PARAGRAPH

The text of the computer readable sequence listing filed herewith, titled “CANAL_44852_202_SequenceListing.xml”, created Apr. 9, 2026, having a file size of 246,416 bytes, is hereby incorporated by reference in its entirety.

### FIELD

Provided herein are compositions, kits, systems, and methods for tagmenting duplex nucleic acid molecules with at least partially double-stranded transposons to generate duplex fragments with loop-like adapters at each end. In some embodiments, the at least partially double stranded transposons herein comprise first and second adapter sequences (e.g., that are separated by a cleavable region), or have a first adapter (e.g., with no cleavable region). In certain embodiments, at least one of the loop like adapters are cleaved at a cleavable region to generate a two-part adapter at one end, or at each end, of the duplex fragment. In certain embodiments, such two-part adapter duplex fragments are subjected to amplification and sequenced to generate sequence reads.

### BACKGROUND

Transposon-base NGS library preparations have proven to be very useful in NGS library preparation as they enable fast and efficient workflows (1). Transposons enable simultaneous fragmentation and adapter tagging of input DNA molecules in a single step, in a so called “Tagmentation” reaction. The resulting DNA molecules are ready for PCR amplification and sequencing (see, e.g., U.S. Pat. No. 10,184,122, FIG. 1). Based on the Tn5 transposase tagmentation principle, a series of commercial products (under name “Nextera”) have been introduced by vendors such as Epicentre Biotechnologies and later Illumina. Other Tn5-based NGS workflows that address a variety of applications soon followed. For examples, by introducing biotin onto the transposon, Mate-pair sequencing was developed for long-range sequencing (2); ATAC-seq was developed to map the accessible chromatin information (3); Tn5mC-seq was developed to map DNA modification information (4).

Although the tagmentation methodology efficiently and effectively generates NGS libraries, there remain some distinct disadvantages to standard Transposon-based methods. The first disadvantage includes loss of template material due to the challenge of attaching at opposite ends of DNA molecules two different adapter sequences (adapter A and adapter B), which is required to successfully amplify, cluster and sequence NGS libraries. In a standard Nextera library preparation, typically only 50% of tagmented molecules will consist of adapter A and adapter B tagged molecules, and 50% of molecules will only have adapter A or adapter B sequences attached on both sides (FIG. 3A). Molecules that lack a combination of adapter A and B, are not viable sequencing molecules as they will not efficiently amplify or cluster in downstream steps. By introducing multiple barcodes onto transposons, Dip-C sequencing library construction utilizes 20 barcodes, which can potentially reduce the initial loss to 1/20 (4). However, such strategy does not eliminate the root cause of the template loss.

A second disadvantage comes from the need to use a polymerase enzyme to complete the addition of adapter sequences at the 3′ end of tagmented DNA strands (FIG. 3B) via an extension step. The inclusion of a polymerase at this stage “writes” directly to the input DNA sample and such writing can lead to the introduction of base replication errors during polymerase extension. After sequencing, such errors could be interpreted as mutations present in the input DNA sample. In addition, the re-synthesized DNA during polymerase extension is void of any biological modification, such as 5-methylcytosine, potentially leading to loss of or wrong estimation of methylation information. The DNA resynthesis can happen both at the 3′ ends of molecules and even internally to a molecule if DNA is damaged with nicks (5).

### SUMMARY

Provided herein are compositions, kits, systems, and methods for tagmenting duplex nucleic acid molecules (e.g., DNA) with at least partially double-stranded transposons to generate duplex fragments with loop-like adapters at each end. In some embodiments, the at least partially double stranded transposons herein comprise first and second adapter sequences (e.g., that are separated by a cleavable region), or have a first adapter (e.g., with no cleavable region). In certain embodiments, at least one of the loop like adapters are cleaved at a cleavable region to generate a two-part adapter at one end, or at each end, of the duplex fragment. In certain embodiments, such two-part adapter duplex fragments are subjected to amplification and sequenced to generate sequence reads. In some embodiments, the methods herein are employed for generating sequencing libraries for diagnosing diseases, such as cancer.

In some embodiments, provided herein are methods comprising: a) generating a mixture that comprises a nucleic acid duplex molecule and a first transposome complex, wherein the first transposome complex comprises: i) a first transposase, ii) a first transposon strand comprising: A) a first adapter sequence comprising a 5′ end and free 3′ end, and a first transposon sequence, B) a first cleavable region, and C) a second adapter sequence comprising a free 5′ end and 3′ end, wherein the first cleavable region is between the first and second adapter sequences; and iii) a second transposon strand comprising a sequence all or partially complementary to the first transposon sequence; and b) incubating the mixture at a temperature such that the nucleic acid duplex molecule and the first transposome complex participate in a tagmentation reaction whereby the first transposase: i) cleaves the nucleic acid duplex molecule creating a duplex fragment that comprises a first duplex end which comprise a free 3′ end and a free 5′ end, and ii) ligates the free 3′ end of the first adapter sequence to the free 5′ end of the first end of the duplex fragment.

In particular embodiments, the methods further comprise: c) contacting the mixture with a ligase enzyme at a temperature such that the free 5′ end of the second adapter sequence is ligated to the free 3′ end of the first end of the duplex fragment, thereby generating a first end loop-like structure at the first end of the duplex fragment. In additional embodiments, the methods further comprise: d) contacting the mixture with one or more enzymes such that the first cleavable region is cleaved thereby generating a first two-part adaptor at the first end of the duplex fragment. In certain embodiments, the method further comprise: c) incubating the mixture at a temperature such that the duplex fragment strands separate to form at least a single-stranded tagged fragment with a free 5′ end and free 3′ end; and d) contacting the mixture with a ligase enzyme at a temperature such that the free 5′ end of the single-stranded tagged fragment is ligated to the free 3′ end of the single-stranded tagged fragment to generate a circularized structure; and optionally further comprising: d) contacting the mixture with one or more enzymes such that the first cleavable region is cleaved in the circularized structure, thereby generating a single stranded dual-tagged construct.

In some embodiments, the mixture in a) further comprises a second transposome complex, wherein the second transposome complex comprises: i) a second transposase, ii) a third transposon strand comprising: A) a third adapter sequence comprising a 5′ end and a free 3′ end, and a second transposon sequence, B) a second cleavable region, and C) a fourth adapter sequence comprising a free 5′ end and 3′ end, wherein the second cleavable region is between the third and fourth adapter sequences; and iii) a fourth transposon strand comprising a sequence all or partially complementary to the third transposon sequence. In further embodiments, the duplex fragment comprises a target insert sequence, wherein the target insert sequence optionally comprises a mutation of interest, wherein the mutation of interest is optionally a cancer-related mutation and/or a SNV, insertion, or deletion, or rearrangement. In certain embodiments, the first and second transposome complexes are identical; and/or wherein the first and third transposon strands are the same or are different; and/or wherein the second and fourth transposon strands are the same or different. In some embodiments, the incubating in step b) causes the second transposome complex to participate in a tagmentation reaction whereby the second transposase: iii) cleaves the duplex fragment creating a second duplex end which comprise a free 3′ end and a free 5′ end, and iv) ligates the free 3′ end of the third adapter sequence of the third transposon strand to the free 5′ end of the second duplex end of the duplex fragment.

In particular embodiments, provided herein are methods comprising: a) generating a mixture that comprises a plurality of nucleic acid duplex molecules and a plurality of transposome complexes, wherein each of the transposome complexes comprises: i) a transposase, ii) a first transposon strand comprising: A) a first adapter sequence comprising a 5′ end and a free 3′ end, and a first transposon sequence, B) a cleavable region, and C) a second adapter sequence comprising a free 5′ end and 3′ end, wherein the cleavable region is between the first and second adapter sequences; and iii) a second transposon strand comprising a sequence all or partially complementary to the first transposon sequence; and b) incubating the mixture at a temperature such that the plurality of nucleic acid duplex molecules and the plurality of transposome complexes participate in tagmentation reactions whereby a plurality of dual-tagged fragments are generated, wherein each of the dual-tagged fragments comprises: i) a double-stranded fragment of a nucleic acid duplex molecule with a first 5′ end and a first 3′ end, and a second 5′ end and a second 3′ end, ii) a first transposon strand is ligated to the first 5′ end of the double-stranded fragment, and iii) a first transposon strand is ligated to the second 5′ end of the double-stranded fragment; and c) contacting the mixture with a ligase enzyme at a temperature such that the first transposon strands previously ligated in step b) are ligated to the first and second 3′ ends of the double-stranded fragment thereby generating a barbell (aka dumbbell) like structure comprising a loop-like structure at both ends. In other embodiments, the methods further comprise: d) contacting the mixture with one or more enzymes such that the cleavable regions are cleaved thereby generating duplex structures with two part adaptors at the first and second ends.

In certain embodiments, provided herein are methods comprising: a) generating a mixture that comprises a nucleic acid duplex molecule and a first transposome complex, wherein the first transposome complex comprises: i) a first transposase, ii) a first transposon strand comprising: A) a first adapter sequence comprising a 5′ end and free 3′ end, and a first transposon sequence, and B) a second adapter sequence comprising a free 5′ end and 3′ end; and iii) a second transposon strand comprising a sequence all or partially complementary to the first transposon sequence; b) incubating the mixture at a temperature such that the nucleic acid duplex molecule and the first transposome complex participate in a tagmentation reaction whereby the first transposase: i) cleaves the nucleic acid duplex molecule creating a duplex fragment that comprises a first duplex end which comprise a 3′ end and a 5′ end, and ii) ligates the 3′ end end of the first adapter sequence to the 5′ end of the first end of the duplex fragment; and c) contacting the mixture with a ligase enzyme at a temperature such that the 5′ end of the second adapter sequence is ligated to the 3′ end of the first end of the duplex fragment, thereby generating a first end loop-like structure at the first end of the duplex fragment. In further embodiments, the methods further comprise: amplifying the end loop-containing duplex fragment using rolling circle amplification.

In some embodiments, the contacting the mixture with the ligase enzyme at the temperature such that the 5′ end of the fourth adapter sequence is ligated to the free 3′ end of the second duplex end of the duplex fragment, thereby generating a second end loop-like structure at the second duplex end of the duplex fragment. In other embodiments, the methods further comprise: d) contacting the mixture with one or more enzymes such that the first and second cleavable regions of the first and third transposon strands are cleaved thereby generating: i) a first two-part adapter at the first duplex end of the duplex fragment that comprises the first and second adapter sequences, and ii) a second two-part adapter at the second duplex end of the duplex fragment that comprises the third and fourth adapter sequences.

In particular embodiments, the first and second adapter sequences of the first two-part adapter do not hybridize, or do not substantially hybridize, with each other; and/or wherein the third and fourth adapter sequences of the second two-part adapter do not hybridize, or do not substantially hybridize, with each other. In other embodiments, each of the first and second two-part adapters have a general Y-shape or V-shape.

In some embodiments, the free 5′ end of the first end of the duplex fragment generated by the cleavage is a first single-stranded protruding end that extends past the free 3′ end of the first end of the duplex fragment, and is exactly, or is at least, 1 or 2 nucleotides in length, and optionally, is exactly, or is at least, 3, 4, 5, 6, 7, 8, 9, 10, 11, or 12 nucleotides in length; and/or wherein the first single-stranded protruding end comprises at least one cytosine that is methylated. In other embodiments, the free 3′ end of the first end of the duplex fragment that is generated by the cleavage is a first single-stranded protruding end that extends past the free 5′ end of the first end of the duplex fragment, and is exactly, or is at least, 1 or 2 nucleotides in length, and optionally, is exactly, or is at least, 3, 4, 5, 6, 7, 8, 9, 10, 11, or 12 nucleotides in length; and/or wherein the first single-stranded protruding end comprises at least one cytosine that is methylated.

In other embodiments, wherein: A) the free 5′ end of the second end of the duplex fragment is a second single-stranded protruding end that extends past the free 3′ end of the second end of the duplex fragment, and is exactly, or is at least, 1 or 2 nucleotides in length, and optionally, is exactly, or is at least, 3, 4, 5, 6, 7, 8, 9, 10, 11, or 12 nucleotides in length; and/or wherein the second single-stranded protruding end comprises at least one cytosine that is methylated; or B) the free 3′ end of the second end of the duplex fragment is a second single-stranded protruding end that extends past the free 5′ end of the second end of the duplex fragment, and is exactly, or is at least, 1 or 2 nucleotides in length, and optionally, is exactly, or is at least, 3, 4, 5, 6, 7, 8, 9, 10, 11, or 12 nucleotides in length; and/or wherein the second single-stranded protruding end comprises at least one cytosine that is methylated. In additional embodiments, the first and/or third transposon sequence comprises a 3′ end transposon end sequence. In certain embodiments, the methods further comprise: amplifying the duplex fragment by adding a plurality of first and second primers and a polymerase to the mixture, wherein the first primers generate a plurality of first amplicons from the duplex fragment, and wherein the second primers generate a plurality of second amplicons from the first amplicons, wherein each of the first primers comprises a 3′ region that at least partially hybridizes to the second adapter sequence and/or the fourth adapter sequence, and wherein each of the second primers comprises a 3′ region that at least partially hybridizes to the complement of the first and/or third adapter sequences. In some embodiments, the methods further comprise: sequencing at least a portion of the first and second amplicons to generate a plurality of sequencing reads. In particular embodiments, the first and/or second primers further comprise at least one element selected from: a flow cell attachment sequence, a unique barcode sequence, a non-unique barcode sequence, a sample-identifying index sequence, and a universal PCR amplification primer binding sequence.

In other embodiments, the first and/or second cleavable regions comprises: A) a modified non-canonical base, which is optionally deoxyuridine or deoxyinosine, B) a nucleic acid sequence backbone linkage, which is optionally a phosphorothioate or a phosphodiester bond, or C) an endonuclease recognition site, wherein the endonuclease recognition site is formed at the junction of the 5′ end of the first or third adapter sequence and the 3′ end of the second or fourth adapter sequence, or is composed of a sequence between the first and second adapter sequences, and/or third and fourth adaptor sequences, or D) one or more RNA bases, wherein optionally the rest of the first and third transposon strands are composed of DNA, or E) comprises a sequence that forms a endonuclease recognition sequence when a secondary oligonucleotide is added. In certain embodiments, no end-repair and/or A-tailing is performed on the duplex fragment. In some embodiments, the temperature of the incubating in c) is between about 55 and 80 degrees Celsius, or between about 60 and 70 degrees Celsius, or is about 60 degrees Celsius, or is about 35-50 degrees Celsius, or is about 49 degrees Celsius. In additional embodiments, the ligase enzyme is selected from: Circligase I, Circligase II, Mth DNA ligase, RtcB ligase from E. coli, thermostable RtcB, LoopLigase, Tph ssDNA ligase SDL, T4 DNA ligase, Hyper-ligase, Taq DNA ligase, T4 RNA ligase 1, and T4 RNA ligase 2. In other embodiments, the ligase enzyme comprises a single strand ligase, and optionally, wherein the single strand ligase has step 3 ligase activity, but not step 2 adenylyl transfer activity, and the free 5′ ends of the second or fourth adaptor sequences of the first or third transposon strand are adenylated. In further embodiments, the single strand ligase is a thermostable lysine-mutant ssDNA/RNA ligase. In other embodiments, the thermostable lysine-mutant is a mutated version of a precursor thermostable ssDNA/RNA ligase, wherein the precursor thermostable ssDNA/RNA ligase has a Motif I EKx(D/N/H)G, and wherein the thermostable lysine-mutant replaces K in the Motif I with any other amino acid or is selected from alanine (A), serine (S), cysteine (C), valine (V), threonine (T), and Glycine (G). In further embodiments, the single strand ligase has an amino acid sequence that is at least 95% or 99% or 100% identical to any one of SEQ ID Nos:23-43.

In certain embodiments, the nucleic acid duplex molecule comprises genomic DNA, cell-free DNA (cfDNA), RNA, an RNA/DNA hybrid, or cDNA, and optionally, wherein the nucleic acid duplex is from a subject. In further embodiments, the subject is a human (e.g., with cancer), and optionally wherein the nucleic acid duplex molecule is extracted from tissue or a bodily fluid, wherein aid bodily fluid is optionally plasma, urine, blood, or CSF fluid. In other embodiments, the free 5′ end of the second adapter is non-phosphorylated, and/or wherein the free 5′ end of the second adapter comprises a blocking group, and wherein, before step c), the mixture is contacted with a phosphorylating or deblocking agent, and/or wherein the deblocking agent is selected from: an enzyme that has phosphorylation activity, which is optionally T4 polynucleotide kinase.

In particular embodiments, the first and/or second adapter sequences, further comprise at least one element selected from: a flow cell attachment sequence, a unique barcode sequence, a non-unique barcode sequence, a sample-identifying index sequence, and a universal PCR amplification primer binding sequence. In other embodiments, the first and/or second adapter sequences comprise a read 1 sequencing primer binding sequence (e.g., all of or part of, SEQ ID NOs:44 or 46) or a read 2 sequencing primer binding sequence (e.g., all of, or part of, SEQ ID NOs:45 or 47). In other embodiments, the third and/or fourth adapter sequences comprise a read 1 sequencing primer binding sequence or a read 2 sequencing primer binding sequence. In some embodiments, the first and second adapter sequences, and optionally the third and fourth adapter sequences, each comprise barcode sequences, and optionally wherein the barcodes sequences have a predefined relationship to each other, and optionally wherein the barcodes sequences are non-complementary to each other, and optionally where the barcode sequences are of different lengths, and optionally where the barcode sequences are same. In additional embodiments, the first adapter sequence comprises a first barcode and the second adapter sequence comprises a second barcode, and wherein some of the plurality of reads comprise a sequence of the first barcode and some of the plurality of reads comprise a sequence of the second barcode; and optionally wherein third adapter sequence comprises a third barcode and the fourth adapter sequences comprises a fourth barcode, and wherein some of the plurality of reads comprise a sequence of the first barcode, and optionally the third barcode, and some of the plurality of reads comprise a sequence of the second barcode, and optionally the fourth barcode. In further embodiments, the methods further comprise: grouping the read based at least in part based on possessing same the first and/or fourth barcode sequences to generate a top strand family, the second and/or third to generate a bottom strand family, wherein the top strand and bottom strand families represent complementary strands which originate from the same the nucleic acid duplex molecule.

In further embodiments, the one more enzymes are selected from: an endonuclease, endonuclease V, endonuclease VIII, an endonuclease and uracil DNA glycosylase, thermostable oxoguanine glycosylase (OGG), and iodine. In other embodiments, the first and/or second transposases are selected from Tn5 transposase, Sleeping Beauty (SB) transposase, Vibrio harveyi, MuA transposase and a Mu transposase recognition site comprising R1 and R2 end sequences, Staphylococcus aureus Tn552, Ty1, Tn7 transposase, Tn/O and IS10, Mariner transposase, Tc1, P Element, Tn3, bacterial insertion sequences, retroviruses, retrotransposon of yeast, IS5, Tn10, Tn903, and IS911. In other embodiments, the first and/or second transposon sequence is from an organism selected from any of the organisms recited above or herein, and optionally wherein the first and/or second 3′ end transposon end sequence are Mosaic ends from Tn5.

In some embodiments, provided herein are compositions (e.g., aqueous compositions) comprising: a first transposon strand comprising: i) a first adapter sequence comprising a 5′ end and a free 3′ end, and a first transposon sequence, ii) a first cleavable region, and iii) a second adapter sequence comprising a free 5′ end and 3′ end, wherein the first cleavable region is between the first and second adapter sequences. In some embodiments, the compositions further comprise: a second transposon strand comprising a sequence all or partially complementary to the first transposon sequence. In other embodiments, the compositions further comprise a first transposase; and/or wherein the first transposon sequence comprises a 3′ end transposon end sequence. In other embodiments, the compositions further comprise a nucleic acid duplex molecule, which is optionally genomic DNA, RNA, RNA/DNA hybrid, cDNA, or cfDNA, or ctDNA. In other embodiments, the compositions further comprise an aqueous buffer.

In some embodiments, the compositions further comprise: a ligase enzyme selected from: Circligase II, Mth DNA ligase, RtcB ligase from E. coli., thermostable RtcB, LoopLigase, Tph ssDNA ligase SDL, T4 DNA ligase, Hyper-ligase, Taq DNA ligase, T4 RNA ligase 1, and T4 RNA ligase 2, optionally selected from ssDNA/RNA ligase mutant wherein the single strand ligase has step 3 ligase activity, but not step 2 adenylyl transfer activity. In other embodiments, the compositions further comprise: one or more enzymes that cleave the cleavable region of the first adapter sequence

In some embodiments, the compositions further comprise: a third transposon strand comprising: i) a third adapter sequence comprising a 5′ end and a free 3′ end, and a second transposon sequence, ii) a second cleavable region, and iii) a fourth adapter sequence comprising a free 5′ end and 3′ end, wherein the second cleavable region is between the third and fourth adapter sequences, and optionally wherein the third transposon strand has a different sequence than the first transposon strand. In certain embodiments, the compositions further comprise: a fourth transposon strand comprising a sequence all or partially complementary to the third transposon sequence. In additional embodiments, the compositions further comprise a second transposase, and optionally that is different from the first transposase. In other embodiments, the first and second adapter sequences do not, or do not substantially, hybridize to each other.

In some embodiments, the compositions further comprise: a polymerase and/or a plurality of first primers, wherein each of the first primers comprises a 3′ region that at least partially hybridizes to the first or second adapter sequence. In other embodiments, the first primers further comprise at least one element selected from: a flow cell attachment sequence, a unique barcode sequence, a non-unique barcode sequence, a sample-identifying index sequence, and a universal PCR amplification primer binding sequence. In further embodiments, the first and/or second cleavable region comprises: A) a modified non-canonical base, which is optionally deoxy-uridine, B) a nucleic acid sequence backbone linkage, which is optionally a phosphorothioate or a phosphodiester bond, or C) an endonuclease recognition site, wherein the endonuclease recognition site is formed at the junction of the first adaptor 5′ end and the second adapter 3′ end, or is composed of a sequence between the first adaptor 5′ end and second adaptor 3′ end, or D) one or more RNA bases, wherein optionally the rest of the first and/or second transposon strands are composed of DNA, or E) comprises a sequence that forms a endonuclease recognition sequence when a secondary oligonucleotide is added.

In particular embodiments, the ligase comprises Circligase I. In other embodiments, the ligase enzyme comprises a single strand ligase. In other embodiments, the single strand ligase is a thermostable lysine-mutant ssDNA/RNA ligase. In further embodiments, the thermostable lysine-mutant is a mutated version of a precursor thermostable ssDNA/RNA ligase, wherein the precursor thermostable ssDNA/RNA ligase has a Motif I EKx(D/N/H)G, and wherein the thermostable lysine-mutant replaces K in the Motif I with any other amino acid or is selected from alanine (A), serine (S), cysteine (C), valine (V), threonine (T), and Glycine (G). In some embodiments, the single strand ligase has an amino acid sequence that is at least 95% or 99% or 100% identical to any one of SEQ ID Nos:23-43.

In certain embodiments, the free 5′ end of the second adapter sequence is un-phosphorylated, and/or wherein the 5′ end of the second adapter sequence comprises a blocking group. In other embodiments, the compositions further comprise a deblocking agent. In particular embodiments, the deblocking agent is selected from: an enzyme that has phosphorylation activity, which is optionally T4 polynucleotide kinase.

In further embodiments, the first and/or second adapter sequences further comprise at least one element selected from: a flow cell attachment sequence, a unique barcode sequence, a non-unique barcode sequence, a sample-identifying index sequence, and a universal PCR amplification primer binding sequence. In certain embodiments, the first adapter sequence comprises a read 1 sequencing primer binding sequence or a read 2 sequencing primer binding sequence. In other embodiments, the second adapter sequence comprises a read 1 sequencing primer binding sequence or a read 2 sequencing primer binding sequence. In other embodiments, the first and second adapter sequences each comprise barcode sequences, and optionally wherein the barcodes sequences have a predefined relationship to each other, and optionally wherein the barcodes sequences are non-complementary to each other, and optionally where the barcodes are of different lengths, and optionally where the barcodes are same. In some embodiments, the first adapter sequence comprises a first barcode and the second adapter sequence comprises a second barcode that is different from the first barcode.

In particular embodiments, the compositions further comprises one more enzymes are selected from: an endonuclease, endonuclease V, endonuclease VIII, an endonuclease and uracil DNA glycosylase, thermostable oxoguanine glycosylase (OGG). In other embodiments, the first and/or second transposases are selected from Tn5 transposase, Sleeping Beauty (SB) transposase, Vibrio harveyi, MuA transposase and a Mu transposase recognition site comprising R1 and R2 end sequences, Staphylococcus aureus Tn552, Ty1, Tn7 transposase, Tn/O and IS10, Mariner transposase, Tc1, P Element, Tn3, bacterial insertion sequences, retroviruses, retrotransposon of yeast, IS5, Tn10, Tn903, and IS911. In other embodiments, the first and/or second transposon sequence is from an organism selected from any of the organisms recited above or herein, and optionally wherein the first and/or second transposon sequence are Mosaic ends from Tn5.

In additional embodiments, provided herein are kits and system comprising: a) a first transposon strand comprising: i) a first adapter sequence comprising a 5′ end and a free 3′ end, and a first transposon sequence, ii) a first cleavable region, and iii) a second adapter sequence comprising a free 5′ end and 3′ end, wherein the first cleavable region is between the first and second adapter sequences; and b) at least one of the following: i) a transposase, ii) a second transposon strand comprising a sequence all or partially complementary to the first transposon sequence, iii) a nucleic acid duplex molecule, which is optionally genomic DNA, or RNA, or RNA/DNA hybrid, or cfDNA, or ctDNA; iv) an aqueous buffer; v) a ligase enzyme, vi) one or more enzymes that cleave the cleavable region; vii) a polymerase; viii) a plurality of first primers, wherein each of the first primers comprises a 3′ region that at least partially hybridizes to the first or second adapter sequence; and ix) one or more containers configured to house any one, all of, the components recited above (e.g., individual vials, a shipping container or wrapper, etc.).

In certain embodiments, the first and second adapter sequences do not, or do not substantially, hybridize to each other. In other embodiments, the first primers are present and further comprise at least one element selected from: a flow cell attachment sequence, a unique barcode sequence, a non-unique barcode sequence, a sample-identifying index sequence, and a universal PCR amplification primer binding sequence. In particular embodiments, the cleavable region comprises: A) a modified non-canonical base, which is optionally deoxyuridine, B) a nucleic acid sequence backbone linkage, which is optionally a phosphorothioate or a phosphodiester bond, or C) an endonuclease recognition site, wherein the endonuclease recognition site is formed at the junction of the first adapter 5′ end and the second adaptor 3′ end, or is composed of a sequence between the first and second adapter, or D) one or more RNA bases, wherein optionally the rest of the first and/or second transposon strands are composed of DNA, or E) comprises a sequence that forms a endonuclease recognition sequence when a secondary oligonucleotide is added.

In further embodiments, the ligase enzyme is present and is selected from: Circligase I, Circligase II, Mth DNA ligase, RtcB ligase from E. coli., thermostable RtcB, LoopLigase, Tph ssDNA ligase SDL, T4 DNA ligase, Hyper-ligase, Taq DNA ligase, T4 RNA ligase 1, and T4 RNA ligase 2. In other embodiments, the ligase enzyme is present and comprises a single strand ligase, and optionally wherein the single strand ligase has step 3 ligase activity, but not step 2 adenylyl transfer activity. In further embodiments, the single strand ligase is a thermostable lysine-mutant ssDNA/RNA ligase. In other embodiments, the thermostable lysine-mutant is a mutated version of a precursor thermostable ssDNA/RNA ligase, wherein the precursor thermostable ssDNA/RNA ligase has a Motif I EKx(D/N/H)G, and wherein the thermostable lysine-mutant replaces K in the Motif I with any other amino acid or is selected from alanine (A), serine (S), cysteine (C), valine (V), threonine (T), and Glycine (G). In additional embodiments, the single strand ligase has an amino acid sequence that is at least 95% or 99% or 100% identical to any one of SEQ ID Nos:23-43.

In some embodiments, the 5′ end of the second adapter sequence is un-phosphorylated, and/or wherein the 5′ end of the second adapter sequence comprises a blocking group. In certain embodiments, the kits and systems further comprise: a deblocking agent. In further embodiments, the deblocking agent is selected from: an enzyme that has phosphorylation activity, which is optionally T4 polynucleotide kinase.

In other embodiments, the first and/or second adapter sequences further comprise at least one element selected from: a flow cell attachment sequence, a unique barcode sequence, a non-unique barcode sequence, a sample-identifying index sequence, and a universal PCR amplification primer binding sequence. In certain embodiments, the first adapter sequence comprises a read 1 sequencing primer binding sequence or a read 2 sequencing primer binding sequence. In other embodiments, the second adapter sequence comprises a read 1 sequencing primer binding sequence or a read 2 sequencing primer binding sequence.

In additional embodiments, the first and second adapter sequences each comprise barcode sequences, and optionally wherein the barcodes sequences have a predefined relationship to each other, and optionally wherein the barcodes sequences are non-complementary to each other, and optionally where the barcodes are of different lengths, and optionally where the barcodes are same. In other embodiments, the first adapter sequence comprises a first barcode and the second adapter sequence comprises a second barcode that is different from the first barcode. In particular embodiments, the one more enzymes are present and are selected from: an endonuclease, endonuclease V, endonuclease VIII, an endonuclease and uracil DNA glycosylase, and thermostable oxoguanine glycosylase (OGG).

In other embodiments, the transposase is present and is selected from Tn5 transposase, Sleeping Beauty (SB) transposase, Vibrio harveyi, MuA transposase and a Mu transposase recognition site comprising R1 and R2 end sequences, Staphylococcus aureus Tn552, Ty1, Tn7 transposase, Tn/O and IS10, Mariner transposase, Tc1, P Element, Tn3, 5 bacterial insertion sequences, retroviruses, retrotransposon of yeast, IS5, Tn10, Tn903, and IS911. In certain embodiments, the transposon sequence is from an organism selected from any of the organisms recited in claim 88, and optionally wherein the 3′ end transposon end sequence is a Mosaic end from Tn5, and optionally wherein the transposon sequence comprises a 3′ end transposon end sequence. In additional embodiments, the ligase enzyme is present and is a single strand ligase, and optionally wherein the single strand ligase has step 3 ligase activity, but not step 2 adenylyl transfer activity.

In some embodiments, provided herein are methods comprising: a) generating a mixture that comprises a nucleic acid duplex molecule and a first transposome complex, wherein the nucleic acid duplex (e.g., cell-free DNA for tumor detection) comprises a top and bottom strand target sequences, wherein the first transposome complex comprises: i) a first transposase, ii) a first transposon strand comprising: a first adapter sequence comprising free 5′ and 3′ ends and a first transposon sequence; and iii) a second transposon strand comprising a sequence all or partially complementary to the first transposon sequence; b) incubating the mixture at a temperature such that the nucleic acid duplex molecule and the first transposome complex participate in a tagmentation reaction whereby the first transposase: i) cleaves the nucleic acid duplex molecule creating a duplex fragment that comprises a first duplex end which comprise a free 3′ end and a free 5′ end, and ii) ligates the free 3′ end of the first adapter sequence to the free 5′ end of the first end of the duplex fragment; c) contacting the mixture with a ligase enzyme (e.g., single strand ligase) at a temperature such that the free 5′ end of the first adapter sequence is ligated to the free 3′ end of the first end of the duplex fragment, thereby generating a first end loop-like structure at the first end of the duplex fragment.

In some embodiments, the mixture in a) further comprises a second transposome complex, wherein the second transposome complex comprises: i) a second transposase, ii) a third transposon strand comprising: A) a second adapter sequence comprising a 5′ end and a free 3′ end, and a second transposon sequence, B) a cleavable region, and C) a third adapter sequence comprising a free 5′ end and 3′ end, wherein the cleavable region is between the second and third adapter sequences; and iii) a fourth transposon strand comprising a sequence all or partially complementary to the second transposon sequence. In some embodiments, the incubating in step b) causes the second transposome complex to participate in a tagmentation reaction whereby the second transposase: iii) cleaves the duplex fragment creating a second duplex end which comprise a free 3′ end and a free 5′ end, and iv) ligates the free 3′ end of the second adapter sequence of the third transposon strand to the free 5′ end of the second duplex end of the duplex fragment. In some embodiments, the contacting the mixture with the ligase enzyme at the temperature such that the 5′ end of the third adapter sequence is ligated to the free 3′ end of the second duplex end of the duplex fragment, thereby generating a second end loop-like structure at the second duplex end of the duplex fragment. In some embodiments, the methods further comprise: d) contacting the mixture with one or more enzymes (e.g., UDG and endo VIII) such that the cleavable region of the third transposon strand is cleaved thereby generating a branched-end hairpin-tethered duplex molecule comprising: i) a two-part adapter at the second duplex end of the duplex fragment that comprises the second and third adapter sequences, and ii) the first end loop-like structure at the first end of the duplex fragment.

In some embodiments, the methods further comprise: contacting the branched-end hairpin-tethered duplex molecule with a primer that hybridizes to the third adapter sequence and performing single-primer extension to generate amplicons, wherein each of the amplicons comprises a copy of the top and bottom target sequences which may or may not have one or more errors introduced. In further embodiments, the methods further comprise: generating an Xpandomer from at least one of the amplicons, and/or directly from the branched-end hairpin-tethered duplex molecule.

In some embodiments, generating the Xpandomer comprises: i) hybridizing the branched-end hairpin-tethered duplex molecule or one of the amplicons to an extension oligo anchored to a surface, ii) extend the extension oligo via an Xp synthase and X-NTPs (expandable nucleotide triphosphates), to generate an extension product, iii) acid treating the extension product to provide cleavage and expansion to generate an Xpandomer, and iv) release the Xpandomer from the surface. In some embodiments, the X-NTPs each comprise a modified dNTP that comprises: i) one of the four canonical bases, ii) a reporter moiety, iii) a translocation control element (TCE), and an acid-cleavable bond. In some embodiments, the methods further comprise: sequencing the Xpandomer (e.g., to identify variants associated with diseases, such as cancer). In some embodiments, the sequencing comprises sequencing by expansion (SBX). In some embodiments, the sequencing by expansion comprises threading the Xpandomer through a Current-Modulation Sensor System to generate sequencing reads. In some embodiments, the methods further comprise: computationally determining the native top and bottom strand target sequences. In some embodiments, the release is by photocleavage.

In some embodiments, the mixture in a) further comprises a second transposome complex. In some embodiments, the mixture in a) further comprises a second transposome complex, wherein the second transposome complex comprises: i) a second transposase, ii) a third transposon strand comprising: a second adapter sequence comprising free 5′ and 3′ ends and a second transposon sequence, wherein the third transposon strand is non-cleavable, except by an enzyme that would also cleave the nucleic acid duplex in separated single-strand form; and iii) a fourth transposon strand comprising a sequence all or partially complementary to the second transposon sequence; wherein the incubating in b) causes the nucleic acid duplex molecule (e.g., cell free DNA, such as tumor cell free DNA) and the second transposome complex participate in a tagmentation reaction whereby the second transposase: i) cleaves the nucleic acid duplex molecule creating a duplex fragment that comprises a second duplex end which comprise a free 3′ end and a free 5′ end, and ii) ligates the free 3′ end of the second adapter sequence to the free 5′ end of the second end of the duplex fragment; and wherein the contacting in c) causes the free 5′ end of the second adapter sequence to be ligated to the free 3′ end of the second end of the duplex fragment, thereby generating a dumbbell template comprising: i) a first end loop-like structure at the first end of the duplex fragment, and ii) a second end loop-like structure at the second end of the duplex fragment.

In some embodiments, the methods further comprise sequencing the dumbbell template. In some embodiments, the methods further comprise: loading the dumbbell template into a zero-mode waveguide (ZMW) such that the dumbbell template is immobilized within the ZMW by association with a polymerase anchored at the base of the ZMW. In some embodiments, the methods further comprise: extending a primer annealed to the dumbbell template by the immobilized polymerase under strand-displacing conditions, thereby generating a continuous nascent strand comprising sequence complementary to both the top and bottom strand target sequences in a single processive synthesis reaction. In some embodiments, the first transposon strand is non-cleavable, except by an enzyme that would also cleave the nucleic acid duplex in separated single-strand form. In some embodiments, the duplex fragment comprises a target insert sequence, wherein the target insert sequence optionally comprises a mutation of interest, wherein the mutation of interest is optionally a cancer-related mutation and/or a SNV, insertion, or deletion, or rearrangement. In some embodiments, the second and third adapter sequences do not hybridize, or do not substantially hybridize, with each other.

In some embodiments, the cleavable region comprises: A) a modified non-canonical base, which is optionally deoxyuridine or deoxyinosine, B) a nucleic acid sequence backbone linkage, which is optionally a phosphorothioate or a phosphodiester bond, or C) an endonuclease recognition site, wherein the endonuclease recognition site is formed at the junction of the 5′ end of the second adapter sequence and the 3′ end of the third adapter sequence, or D) one or more RNA bases, wherein optionally the rest of the first and third transposon strands are composed of DNA, or E) comprises a sequence that forms a endonuclease recognition sequence when a secondary oligonucleotide is added. In some embodiments, the ligase enzyme is selected from: Circligase I, Circligase II, Mth DNA ligase, RtcB ligase from E. coli., thermostable RtcB, LoopLigase, Tph ssDNA ligase SDL, T4 DNA ligase, Hyper-ligase, Taq DNA ligase, T4 RNA ligase 1, and T4 RNA ligase 2. In some embodiments, the ligase enzyme comprises a single strand ligase, and optionally, wherein the single strand ligase has step 3 ligase activity, but not step 2 adenylyl transfer activity, and the free 5′ ends of the first and/or third adaptor sequences are adenylated. In some embodiments, the single strand ligase is a lysine-mutant ssDNA/RNA ligase (e.g., thermostable). In some embodiments, the lysine-mutant (e.g., thermostable) is a mutated version of a precursor ssDNA/RNA ligase, wherein the precursor ssDNA/RNA ligase has a Motif I EKx(D/N/H)G, and wherein the lysine-mutant replaces K in the Motif I with any other amino acid or is selected from alanine (A), serine (S), cysteine (C), valine (V), threonine (T), and Glycine (G).

In some embodiments, the free 5′ end of the first end of the duplex fragment generated by the cleavage is a first single-stranded protruding end that extends past the free 3′ end of the first end of the duplex fragment, and is exactly, or is at least, 1 or 2 nucleotides in length, and optionally, is exactly, or is at least, 3, 4, 5, 6, 7, 8, 9, 10, 11, or 12 nucleotides in length; and/or wherein the first single-stranded protruding end comprises at least one cytosine that is methylated. In some embodiments, the free 3′ end of the first end of the duplex fragment that is generated by the cleavage is a first single-stranded protruding end that extends past the free 5′ end of the first end of the duplex fragment, and is exactly, or is at least, 1 or 2 nucleotides in length, and optionally, is exactly, or is at least, 3, 4, 5, 6, 7, 8, 9, 10, 11, or 12 nucleotides in length; and/or wherein the first single-stranded protruding end comprises at least one cytosine that is methylated.

In certain embodiments, the nucleic acid duplex molecule comprises genomic DNA, cell-free DNA (cfDNA), RNA, an RNA/DNA hybrid, or cDNA, and optionally, wherein the nucleic acid duplex is from a subject. In some embodiments, the subject is a human (e.g., with cancer or potentially with cancer), and optionally wherein the nucleic acid duplex molecule is extracted from tissue or a bodily fluid, wherein aid bodily fluid is optionally plasma, urine, blood, or CSF fluid.

In some embodiments, the free 5′ end of the first adapter sequence is non-phosphorylated, and/or wherein the free 5′ end of the first adapter sequence comprises a blocking group, and wherein, before step c), the mixture is contacted with a phosphorylating or deblocking agent, and/or wherein the deblocking agent is selected from: an enzyme that has phosphorylation activity, which is optionally T4 polynucleotide kinase. In some embodiments, the first and/or second adapter sequences, further comprise at least one element selected from: a flow cell attachment sequence, a unique barcode sequence, a non-unique barcode sequence, a sample-identifying index sequence, and a universal PCR amplification primer binding sequence. In other embodiments, the first and/or second adapter sequences comprise a read 1 sequencing primer binding sequence or a read 2 sequencing primer binding sequence. In some embodiments, the first and second adapter sequences, and optionally the third sequence, each comprise barcode sequences, and optionally wherein the barcodes sequences have a predefined relationship to each other, and optionally wherein the barcodes sequences are non-complementary to each other, and optionally where the barcode sequences are of different lengths, and optionally where the barcode sequences are same. In some embodiments, the second adapter sequence comprises a first barcode and the third adapter sequence comprises a second barcode, and wherein some of the plurality of reads comprise a sequence of the first barcode and some of the plurality of reads comprise a sequence of the second barcode.

In some embodiments, the one more enzymes are selected from: an endonuclease, endonuclease V, endonuclease VIII, an endonuclease and uracil DNA glycosylase, thermostable oxoguanine glycosylase (OGG), and iodine. In some embodiments, the first and/or second transposases are selected from Tn5 transposase, Sleeping Beauty (SB) transposase, Vibrio harveyi, MuA transposase and a Mu transposase recognition site comprising R1 and R2 end sequences, Staphylococcus aureus Tn552, Ty1, Tn7 transposase, Tn/O and IS10, Mariner transposase, Tc1, P Element, Tn3, bacterial insertion sequences, retroviruses, retrotransposon of yeast, IS5, Tn10, Tn903, and IS911. In some embodiments, the first and/or second transposon sequence is from an organism selected from any of the organisms recited in claim 132, and optionally wherein the first and/or second 3′ end transposon end sequence are Mosaic ends from Tn5.

In some embodiments, provided herein are compositions, kits, and systems comprising: a) a first transposon strand comprising: a first adapter sequence comprising free 5′ and 3′ ends and a first transposon sequence, and b) a second transposon strand comprising a sequence all or partially complementary to said first transposon sequence; and c) at least one of the following: i) a transposase, ii) a nucleic acid duplex molecule, which is optionally genomic DNA, or RNA, or RNA/DNA hybrid, or cfDNA, or ctDNA; iii) an aqueous buffer; iv) a ligase enzyme, which is optionally a single strand ligase; v) a polymerase; vi) a plurality of first primers, wherein each of said first primers comprises a 3′ region that at least partially hybridizes to said adapter sequence; and vii) one or more containers configured to house any one, all of, the components recited above.

In some embodiments, the ligase is the single strand ligase which comprises Circligase. In some embodiments, the single strand ligase comprises a lysine-mutant (e.g., thermostable) ssDNA/RNA ligase. In some embodiments, the lysine-mutant (e.g., thermostable) is a mutated version of a precursor ssDNA/RNA ligase, wherein the precursor ssDNA/RNA ligase has a Motif I EKx(D/N/H)G, and wherein the thermostable lysine-mutant replaces K in the Motif I with any other amino acid or is selected from alanine (A), serine (S), cysteine (C), valine (V), threonine (T), and Glycine (G). In some embodiments, the single strand ligase has an amino acid sequence that is at least 95% or 99% or 100% identical to any one of SEQ ID Nos:23-43. In some embodiments, the single strand ligase is selected from: Circligase II, Mth DNA ligase, RtcB ligase from E. coli., thermostable RtcB, LoopLigase, Tph ssDNA ligase SDL, T4 DNA ligase, Hyper-ligase, Taq DNA ligase, T4 RNA ligase 1, and T4 RNA ligase 2, optionally selected from ssDNA/RNA ligase mutant wherein the single strand ligase has step 3 ligase activity, but not step 2 adenylyl transfer activity.

In some embodiments, the compositions, kits, and systems further comprise: i) a nucleic acid duplex molecule, which is optionally genomic DNA, RNA, RNA/DNA hybrid, cDNA, or cfDNA, or ctDNA, and/or ii) an aqueous buffer. In some embodiments, the compositions, kits, and systems further comprise: c) second transposome complex comprises: i) a second transposase, ii) a third transposon strand comprising: A) a second adapter sequence comprising a 5′ end and a free 3′ end and a second transposon sequence, B) a cleavable region, and C) a third adapter sequence comprising a free 5′ end and 3′ end, wherein the cleavable region is between the second and third adapter sequences; and iii) a fourth transposon strand comprising a sequence all or partially complementary to the second transposon sequence. In some embodiments, the second and third adapter sequences do not, or do not substantially, hybridize to each other.

In some embodiments, the compositions, kits, and systems further comprise: one or more enzymes that cleave the cleavable region, which are optionally selected from: an endonuclease, endonuclease V, endonuclease VIII, an endonuclease and uracil DNA glycosylase, thermostable oxoguanine glycosylase (OGG). In some embodiments, the compositions, kits, and systems further comprise a polymerase and/or a plurality of first primers, wherein each of the first primers comprises a 3′ region that at least partially hybridizes to the second or third adapter sequence. In some embodiments, the first primers further comprise at least one element selected from: a flow cell attachment sequence, a unique barcode sequence, a non-unique barcode sequence, a sample-identifying index sequence, and a universal PCR amplification primer binding sequence. In some embodiments, the cleavable region comprises: A) a modified non-canonical base, which is optionally deoxy-uridine, B) a nucleic acid sequence backbone linkage, which is optionally a phosphorothioate or a phosphodiester bond, or C) an endonuclease recognition site, wherein the endonuclease recognition site is formed at the junction of the second adaptor 5′ end and the third adapter 3′ end, or is composed of a sequence between the second adaptor 5′ end and third adaptor 3′ end, or D) one or more RNA bases, wherein optionally the rest of the third and/or fourth transposon strands are composed of DNA, or E) comprises a sequence that forms a endonuclease recognition sequence when a secondary oligonucleotide is added.

In some embodiments, the free 5′ end of the third adapter sequence is un-phosphorylated, and/or wherein the 5′ end of the third adapter sequence comprises a blocking group. In some embodiments, the compositions, kits, and systems, further comprise a deblocking agent, which is optionally selected from: an enzyme that has phosphorylation activity, which is optionally T4 polynucleotide kinase.

In some embodiments, the second and/or third adapter sequences further comprise at least one element selected from: a flow cell attachment sequence, a unique barcode sequence, a non-unique barcode sequence, a sample-identifying index sequence, and a universal PCR amplification primer binding sequence. In some embodiments, the third adapter sequence comprises a read 1 sequencing primer binding sequence or a read 2 sequencing primer binding sequence. In some embodiments, the second and third adapter sequences each comprise barcode sequences, and optionally wherein the barcodes sequences have a predefined relationship to each other, and optionally wherein the barcodes sequences are non-complementary to each other, and optionally where the barcodes are of different lengths, and optionally where the barcodes are same. In some embodiments, the second adapter sequence comprises a first barcode and the third adapter sequence comprises a second barcode that is different from the first barcode. In some embodiments, the first and/or second transposases are selected from Tn5 transposase, Sleeping Beauty (SB) transposase, Vibrio harveyi, MuA transposase and a Mu transposase recognition site comprising R1 and R2 end sequences, Staphylococcus aureus Tn552, Ty1, Tn7 transposase, Tn/O and IS10, Mariner transposase, Tc1, P Element, Tn3, bacterial insertion sequences, retroviruses, retrotransposon of yeast, IS5, Tn10, Tn903, and IS911. In some embodiments, the first and/or second transposon sequence is from an organism selected from any of the organisms recited in claim 154, and optionally wherein the first and/or second transposon sequence are Mosaic ends from Tn5.

In some embodiments, the compositions, kits further comprise: one or more containers configured to house any one, all of, the recited components. In some embodiments, the compositions, kits, and systems further comprise: c) a second transposome complex comprising: i) a second transposase, ii) a third transposon strand comprising: a second adapter sequence comprising free 5′ and 3′ ends and a second transposon sequence; and iii) a fourth transposon strand comprising a sequence all or partially complementary to the second transposon sequence. In some embodiments, the first and second transposome complexes are identical. In some embodiments, the first and second transposome complexes have at least one difference selected from: i) type of transposase, ii) nucleic acid sequence of the adapters sequences, and iii) nucleic acid sequence of the transposon sequences.

### BRIEF DESCRIPTION OF THE DRAWINGS

FIGS. 1A-B. Illustration of an exemplary embodiment of Tagment and Looped Ligation (TagALL) library preparation. FIG. 1A: Example Transposome assembled from Tn5 Transposase and novel transposon oligo design. FIG. 1B: Exemplary Tagmentation and looped ligation workflow to generate NGS libraries.

FIGS. 2A-B. Illustration of an exemplary embodiment of Tagment and Looped Ligation (TagALL) library preparation via Single Stranded circularization. FIG. 2A: Exemplary Transposome assembled from Tn5 Transposase and novel transposon oligo design. FIG. 2B: Exemplary Tagmentation and single strand ligation workflow to generate NGS libraries.

FIGS. 3A-B depicts the challenges of standard “Nextera” Tn5 Transposon-based Library preparation, including: FIG. 3A: inefficient template usage, and FIG. 3B: polymerase extension and gap filling, leading to potential base error incorporation and loss of native methylation signal.

FIG. 4 shows sequence structure of exemplary transposons and schematic representation of exemplary Transposomes.

FIGS. 5A-B shows the impact of adapter sequence composition on looped ligation efficiency, where in FIG. 5A complementary mosaic ends could impede proximal loop ligations, where in FIG. 5B non-complementary ends may have improved proximal loop ligation.

FIG. 6 shows results of exemplary Tagmentation reactions of Human genomic DNA, using several exemplary Transposomes and a control standard Transposon Tagmentation reaction.

FIG. 7 shows an Agilent High Sensitivity Tapestation analysis of exemplary final amplified library products prior to sequencing. Control Transposome based libraries representing industry standard library we generated following the protocol described in the Example below.

FIGS. 8A-E shows various exemplary transposon (Tn) designs iterations which are partially double stranded transposon complexes composed of a first transposon strand and a second transposon strand (e.g., comprising a mosaic end sequence). FIG. 8A shows an exemplary Stubby Tn used in Example 1. In this construct, R1 and R2 sequences are included in the transposon, no UMI (barcode) is included, and samples indexes and clustering sequences could be added by PCR primers. The R1 primer binding sequence in this construct includes both the gray and orange in the top strand, where the grey is a Mosaic sequence (ME) which also forms part of the R1 sequence primer binding site. FIG. 8B shows an exemplary ‘full length Tn’ composed of R1+R2 (sequencing read primer binding sites), i5 and i7 index sites (e.g., for pooling), and P5 and P7 flow cell binding sequences. FIG. 8C shows an exemplary ‘full length Tn+UMI,’ where u5 and u7 are added as barcodes (e.g., 1 to 20 bases in length, which can be random). FIG. 8D shows an exemplary ‘full length Tn+UMI+ME’ construct that places the UMI's inline with R1 and R2, and adds a mosaic end separated from R1. FIG. 8E shows an exemplary ‘stubby Tn+UMI+ME,” that has the UMI's inline with R1 and R2, and adds mosaic end separated from R1.

FIGS. 9A-C shows base composition from standard libraries and from three different TagALL compositions. The differing base composition at the start of R1 and R2 demonstrates that a looped ligation event is successfully being completed. GC percentage base composition (Y-axis) are shown by read cycle (X-axis) for Read 1 and Read 2 for distinct transposon designs. The data shows matched base-composition patterns based on anticipated tagmented molecule ends, and a distinct base-composition pattern at anticipated looped ligation molecule ends. Data is shown for both double stranded looped ligation and single stranded circularizations, base compositions for both workflows are matching for a given transposon. This evidence taken together indicates successful tagmentation and subsequent ligation events from the TagALL transposons. FIG. 9A shows standard Transposon-based library prep. Due to the polymerase “fill-in” step, both read 1 (5′ end) and read 2 (3′-end) show similar base composition. FIG. 9B shows base composition from TagALL TPL1 & TPL2 libraries. Based on TPL1 and TPL2 transposon design, read 1 originates from 5′ tagmentation sites and Read 2 from 3′ looped ligation, for which different base composition can be observed. FIG. 9C shows base composition from TagALL TPL3 library. Based on TPL3 transposon design, read 1 originates from 3′ looped ligation, and Read 2 originates from 5′ tagmentation fragment end, for which base composition patterns switched compared to TPL1 &TPL2 libraries.

FIG. 10A. Exemplary diagram of duplex information recovery using first and second adaptor barcodes with predefined association (see look up tables 5-8). The Duplex DNA fragment shows both ends with a protruding end of about 9 nucleotides. Duplex DNA fragment ends are ligated (by the transposase) with adaptors with first and second adaptor barcodes forming the dumbbell (aka barbell) like DNA molecule shown with the protruding ends that are preserved. The two loops of this dumbbell DNA molecule are then cleaved. PCR amplification is then employed, such as with universal primers. These amplicons are then sequenced to generate sequence reads. Read pairs with proper association are used to recover duplex, as shown in the figure. Note that the recovered duplex is not necessarily blunt ended. The end coordinates can be used to recover the ends present in the starting material.

FIG. 10B shows the same exemplary figure as FIG. 10A, but without barcodes added and with 3′ protruding ends.

FIG. 10C shows a similar exemplary figure without barcodes and 5′ protruding ends.

FIG. 11A. Transposons with barcodes are loaded to Tn5 separately. In this example, as many as 96 transposons with distinct set of barcodes are loaded onto Tn5 and used for tagmentation.

FIG. 11B. The product of TagALL process after using such transposome mixture on a long duplex DNA molecule. At each insertion site, two duplex ends are generated, and each end is attached with the same barcode coming from the transposome.

FIGS. 11C, D and E. Sequenced read pairs with distance of 0 bp(C), −9 bp(D), 9 bp (E), and the same barcode, can be “concatenated” to restore contiguity information.

FIGS. 12A-B. shows an exemplary TagALL library preparation using a mixture of cleavable and un-cleavable transposons. FIG. 12A: An exemplary design of cleavable (based on the modified base deoxyuracil) and un-cleavable transposon. FIG. 12B: After tagmentation, a portion of the genomic DNA fragments will have a cleavable transposon ligated at one end and an un-cleavable transposon ligated at the other end. After proximal duplex ligation (i.e., loop ligation), the same genomic DNA fragments will have a cleavable loop at one end and an un-cleavable loop at the other end. After cleavage, the cleavable loop becomes a two-part adaptor ligated to each duplex strand, while the un-cleavable loop remains and keeps the duplex strands tethered, which can be termed, for example, a branched-end hairpin-tethered duplex molecule (e.g., which may be sequenced with Roche's SBX-D methods).

FIGS. 13A-B shows an exemplary TagALL library preparation using un-cleavable (e.g., not cleavable by enzymes that wouldn't also cleave the target duplex) transposons.

FIG. 13A: An exemplary design of un-cleavable transposon. FIG. 13B: After tagmentation and loop ligation, the genomic fragment has a loop structure at each duplex end, tethering the two duplex strands. Such structures may, for example, be sequenced by PacBio's methods, such as their Single-molecule real-time (SMRT) sequencing (e.g., where they call such closed loop structures SMRTbells™)

FIG. 14 shows the structure of an exemplary TagALL duplex transposon, including the placement of inline duplex UMIs within the adapter sequences and the associated transposon mosaic end (ME) sequence. In this embodiment, the paired UMIs are the same fixed sequence of 6 nucleotides, in a different orientation. A total of 96 distinct duplex transposons were utilized to generate the data described herein (see FIG. 18).

FIG. 15 shows the base composition of sequencing reads generated from a human genomic DNA library prepared using the TagALL duplex 96-UMI transposon pool and sequenced using a 2×75 bp Illumina sequencing run. The observed base composition across read cycles is consistent with successful library construction and confirms the expected read structure.

FIG. 16 shows an example of a duplex molecule composed of two paired-end reads derived from opposite strands, exhibiting: i) read alignments in opposite orientations between strands; ii) matching but oppositely orientated UMIs, and iii) an offset between aligned read positions consistent with transposase-mediated fragmentation. In this example, the observed offset is approximately 9 base pairs, consistent with the expected staggered cleavage pattern generated by Tn5 transposase.

FIG. 17 shows the distribution of alignment offsets for all detected duplex molecules within a representative library. Greater than 60% of identified duplex molecules exhibit an offset of approximately 9 base pairs, consistent with the expected transposase-generated stagger and supporting accurate duplex pairing.

FIG. 18 shows a UMI pairing table and corresponding oligonucleotide sequences for Example 1.

### DEFINITIONS

A single-stranded DNA/RNA ligase enzyme is considered to have “step 3 ligase activity” but not “step 2 adenylyl transfer activity,” when it is able to ligate between 5′-adenylated end and 3′-hydroxyl end of single strand DNA/RNA, but not able to transfer the AMP to the 5′-phosphate-terminated DNA or RNA strand to form a 5′-App-DNA/RNA intermediate. All known DNA and RNA ligases perform the catalysis via a common pathway which involves three nucleotidyl transfer reactions (Lehman et al, Science, 1974; Lindahl et al, Annu Rev Biochem, 1992). In the case of ATP-dependent DNA or RNA ligases, the first step (step 1) involves the attack on the α-phosphate of ATP by ligase, which results in release of pyrophosphate and formation of a ligase-AMP intermediate. AMP is linked covalently to the amino group of a lysine residue within a conserved sequence motif. In the second step (step 2), the AMP nucleotide is transferred to the 5′-phosphate-terminated DNA or RNA strand to form a 5′-App-DNA/RNA intermediate. In the third and final step (step 3), attack by the 3′-OH strand on the 5′-App-DNA/RNA end joins the two polynucleotides and liberates AMP.

“End repair” means fill-in of the 3′-single-strand protruding ends by polymerase and/or resection of the 5′-single-strand protruding ends of the original DNA analyte DNA duplex. In certain embodiments, the methods herein do not employ any type of end repair.

### DETAILED DESCRIPTION

Provided herein are compositions, kits, systems, and methods for tagmenting duplex nucleic acid molecules with at least partially double-stranded transposons to generate duplex fragments with loop-like adapters at each end. In some embodiments, the at least partially double stranded transposons herein comprise first and second adapter sequences (e.g., that are separated by a cleavable region), or have a first adapter (e.g., with no cleavable region). In certain embodiments, at least one of the loop like adapters are cleaved at a cleavable region to generate a two-part adapter at one end, or at each end, of the duplex fragment. In certain embodiments, such two-part adapter duplex fragments are subjected to amplification and sequenced to generate sequence reads.

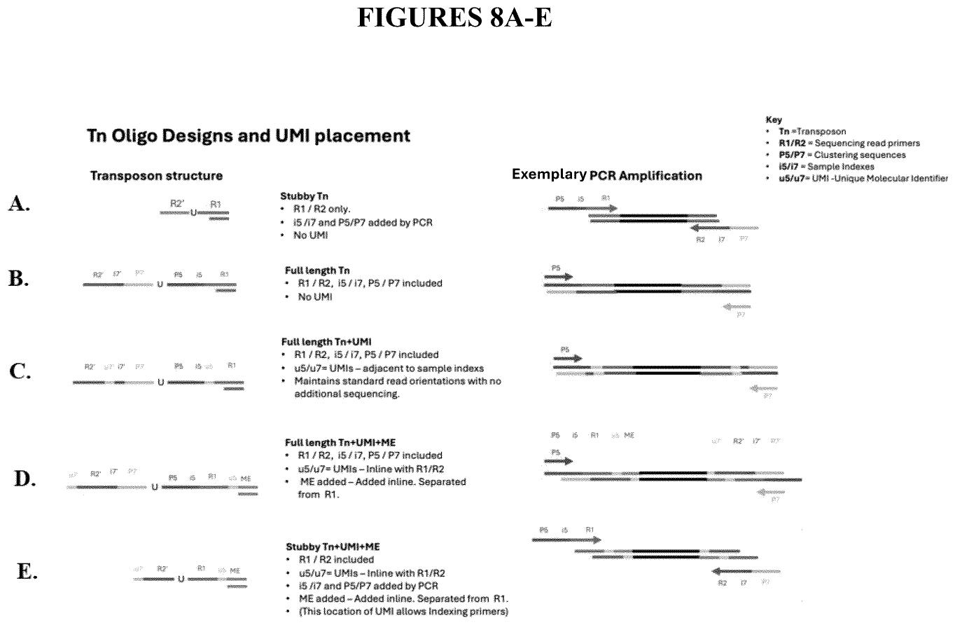

In certain embodiments, the at least partially double stranded transposons herein comprise first and/or second adapter sequences that include a barcode (also known as a UMI, or “unique molecular identifier” that does not necessarily have to be unique). The barcodes can be unique in the population or non-unique, and can be from 1 to about 20 bases in length. Such barcodes allow single molecule tracking and grouping of sequencing reads into families (which allows single strand consensus sequences, that can then be paired into duplex consensus sequences, see, e.g., duplex sequencing methods in U.S. Patents and US Patent Publications: U.S. Pat. Nos. 11,555,220; 11,608,529; 11,584,964; 11,479,807; 2019/0352714; 2023/0193378; and 12,00,6532, all of which are herein incorporated by reference, particularly for sequencing and error correction). Examples of cleavable adapters herein are shown in FIG. 8, most of which contain UMIs (barcodes). FIG. 8A shows a transposon that is composed of R1 and R2 adapter sequences, without any UMI (barcode) sequences (e.g., where samples indexes and clustering sequences can be added by PCR primers). In other embodiments, one could utilize a full length adapter with indexes and clustering sequences present in the starting transposon (e.g., FIGS. 8B-8D). In certain embodiments, such longer adapters would allow the addition of UMI sequences next to the sample index (i5/i7) for example. In other embodiments, the UMIs are ‘inline’ with the read 1 and read 2 sequencing primers (e.g., which could include a transposon ME (Mosaic End) sequence also in-line with sequencing primer at the 5′ end of the transposon). This ME sequence could thus have to be sequenced as part of the insert read, although it is possible to use dark cycles and not image the ME bases. One advantage of using in-line UMIs and ME, is that would enable the use of a Stubby-Tn design, allowing sample index and clustering sequences to be added at PCR stage and thus reducing manufacturing complexity and costs associated with the longer transposons and the need for higher numbers of index and barcode combinations.

In particular embodiments, the transposons herein comprise a first adapter sequence and a second adapter sequence, wherein the first adapter sequence comprises a first barcode sequence, and the second adapter sequence comprises a second barcode sequence. In some embodiments, the first and second barcodes sequences are complementary and may hybridize to each other. In other embodiments, the first and second barcodes are not complementary and do not hybridize to each other.

Such embodiments where the first and second barcode sequences do not hybridize to each other is different than the normal approach in the art where the barcode sequence is in the “stalk” of a Y shaped adaptor, necessitating that the first and second barcodes are always complementary to each other (due to the constraint of base pairing). As the result, in normal approach, adaptor with barcode A ligates to one end of the duplex and adaptor with barcode B ligates to the other end of the duplex, so that read pair from one strand has barcode “AB”, and read pair from the other strand has barcode “BA.” During analysis, read pairs that share the same genomic coordinates of start and stop positions, and also AB/BA barcodes are considered as duplex.

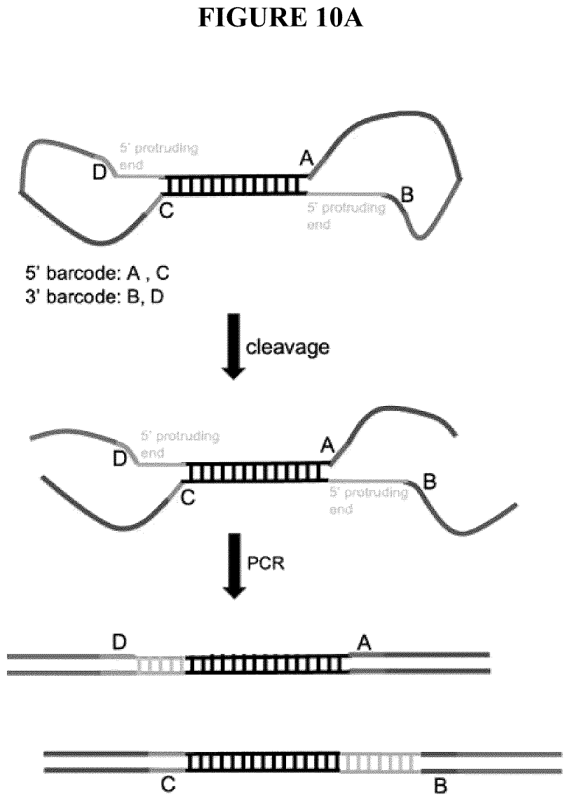

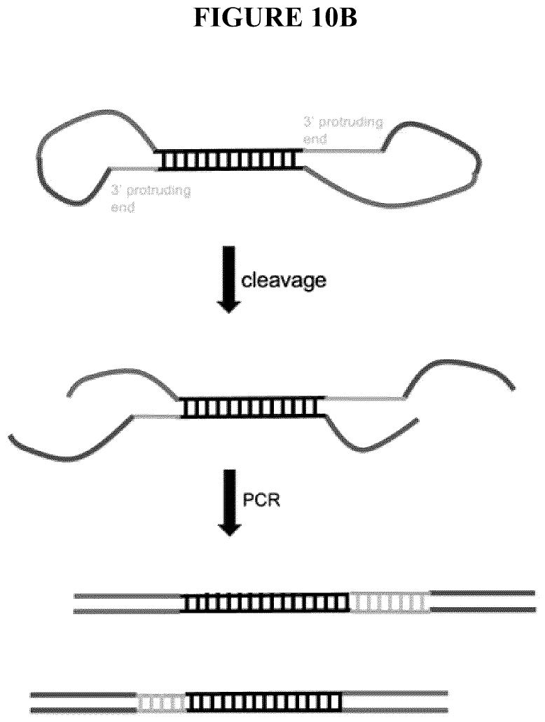

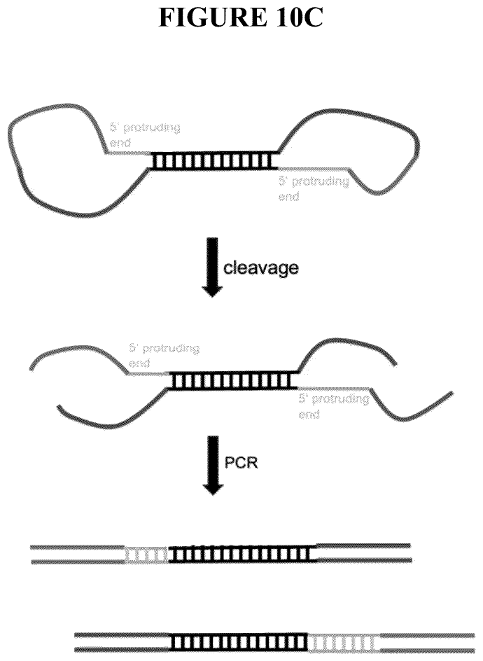

In embodiments herein the first and second adapter barcodes are not complementary to each other, as shown in Table 1 below: where AB, CD, EF, and GH each represent 5′ barcode and 3′ barcode and do not hybridize to each other. First and second adapter sequences with unique combinations of AB, CD, EF, and GH may be synthesized individually, and used as a mixture. As an example, during ligation, first adaptor sequence with barcode A and B ligates to the first duplex end, and second adaptor sequence with barcode C and D ligates to the second duplex end (FIG. 10A). Read pair from one strand has barcodes D and A, and read pair from the other strand has barcodes B and C. During analysis, read pairs that have their start and stop positions differ by approximately 9 bases, and have the corresponding association as defined in the look up table (e.g., as long as they pair with each other in this table, DA/BC in the example, and therefore have a predetermined relationship that can be looked up) are considered duplex. The 9 base gap at the ends is an innate property of the transposase used (e.g., Tn5).

TABLE 1

| 5′ barcode | 3′ barcode |
|---|---|
| A | B |
| C | D |
| E | F |
| G | H |

FIG. 10A provides an exemplary figure demonstrating the use of barcodes that can be looked up (for bottom and top strand) in a look up table such as Table 1 above. FIG. 10A shows that the Duplex DNA fragment (created by cleavage by a transposase) with both ends with a protruding 5′ end of about 9 nucleotides. Duplex DNA fragment ends are ligated with first adapter (by a transposase) and second adaptor (by looped ligation), with barcodes forming the dumbbell (aka barbell like) DNA molecule shown with the protruding template ends that are preserved. The two loops of this dumbbell DNA molecule are then cleaved. PCR amplification is then employed, such as with universal primers. These amplicons are then sequenced to generate sequence reads. Read pairs with proper association are used to recover duplex information, as shown in the figure. Note that the recovered duplex is not necessarily blunt ended.

As discussed above, the barcodes herein do not need to be complimentary, which, for example, expands the design space to 16 possible combinations for 1-mers as shown in Table 2.0 below, in contrast to only 4 possible combinations if complementarity is required.

TABLE 2.0
1-mer duplex barcode design table

| 5′-barcode | 3′-barcode | Mismatch |
|---|---|---|
| A | A | 1 |
| A | C | 1 |
| A | G | 1 |
| A | T | 0 |
| C | C | 1 |
| C | G | 0 |
| C | T | 1 |
| C | A | 1 |
| G | A | 1 |
| G | T | 1 |
| G | C | 0 |
| G | G | 1 |
| T | T | 1 |
| T | A | 0 |
| T | C | 1 |
| T | G | 1 |

A possible look up table (Table 2.1) for the 1 mer duplex barcode is:

TABLE 2.1

| 5′-barcode | 3′-barcode |
|---|---|
| A | A |
| C | T |
| G | G |
| T | C |

Another possible look up table (Table 2.2) for the 1 mer duplex barcode is:

TABLE 2.2

| 5′-barcode | 3′-barcode |
|---|---|
| A | C |
| C | T |
| G | A |
| T | G |

As another example (Table 3), barcodes of equal or unequal length can be used as duplex barcodes.

TABLE 3
barcodes of different length

| 5′-barcode | 3′-barcode |
|---|---|
| AA | CCT |
| CC | TT |
| GC | A |
| TA | CCGG |
| TC | CC |

As another example (Table 4), barcodes of the same sequences can be used as duplex barcodes. This design has the added advantage that a look up table is not even needed.

TABLE 4
an exemplary design of 96 duplex
barcodes with the same sequences

| 5′-Barcode | 3′-Barcode |
|---|---|
| AATGCAC | AATGCAC |
| ATCCTTG | ATCCTTG |
| GTATGCT | GTATGCT |
| AGATGCG | AGATGCG |
| ATGTGGC | ATGTGGC |
| CGATACT | CGATACT |
| GACTGTA | GACTGTA |
| GGTACGT | GGTACGT |
| TAACGCG | TAACGCG |
| TTACGGC | TTACGGC |
| AACCGCA | AACCGCA |
| AACGTCC | AACGTCC |
| ACGCAGT | ACGCAGT |
| AGGCACA | AGGCACA |
| CAACACA | CAACACA |
| GATCGCT | GATCGCT |
| GGATATA | GGATATA |
| GGATCAG | GGATCAG |
| TTGTGCG | TTGTGCG |
| ACCGAAC | ACCGAAC |
| AGCGACT | AGCGACT |
| AGGTTAA | AGGTTAA |
| ATATTGG | ATATTGG |
| ATCACAC | ATCACAC |
| ATTAGGT | ATTAGGT |
| CAATATC | CAATATC |
| CAGTGGA | CAGTGGA |
| CCGATAG | CCGATAG |
| CTACATT | CTACATT |
| CTGTATA | CTGTATA |
| GAGTGAG | GAGTGAG |
| GATCCAG | GATCCAG |
| GCTACAC | GCTACAC |
| GCTCGAA | GCTCGAA |
| GGATTCC | GGATTCC |
| GGTGCAA | GGTGCAA |
| GTAAGAG | GTAAGAG |
| GTACCTG | GTACCTG |
| GTAGTCG | GTAGTCG |
| GTGTACC | GTGTACC |
| TCCGACA | TCCGACA |
| TTAGCCT | TTAGCCT |
| ACTGTTC | ACTGTTC |
| AGAATAC | AGAATAC |
| ATCAATA | ATCAATA |
| ATGCGCT | ATGCGCT |
| ATTGTAG | ATTGTAG |
| CAATGAT | CAATGAT |
| CATACAT | CATACAT |
| CATGCCA | CATGCCA |
| CGCGATA | CGCGATA |
| CGGCAAT | CGGCAAT |
| CTGTTAT | CTGTTAT |
| GAATAGG | GAATAGG |
| GATTATT | GATTATT |
| GCCTTAC | GCCTTAC |
| GTTAGTA | GTTAGTA |
| TACCATA | TACCATA |
| TATAATG | TATAATG |
| TCGAAGG | TCGAAGG |
| TCGACAT | TCGACAT |
| TGAAGTG | TGAAGTG |
| TGTCCAT | TGTCCAT |
| TGTTAAG | TGTTAAG |
| AACTTAG | AACTTAG |
| AAGCTGG | AAGCTGG |
| AATATCG | AATATCG |
| AATGTGT | AATGTGT |
| ACACTTA | ACACTTA |
| ACGAACC | ACGAACC |
| AGGTAGG | AGGTAGG |
| ATAGGCC | ATAGGCC |
| ATATAAC | ATATAAC |
| ATGATTC | ATGATTC |
| CACTCAC | CACTCAC |
| CAGGAAC | CAGGAAC |
| CCGGATT | CCGGATT |
| CGGATGT | CGGATGT |
| CTAATAA | CTAATAA |
| CTCTGTG | CTCTGTG |
| CTGGTGA | CTGGTGA |
| CTTGTCC | CTTGTCC |
| GAGCAGA | GAGCAGA |
| GCAATGG | GCAATGG |
| GCCGTTA | GCCGTTA |
| GCTGAAG | GCTGAAG |
| GTTCTTC | GTTCTTC |
| GTTGGAT | GTTGGAT |
| TAACCTT | TAACCTT |
| TAGCGGT | TAGCGGT |
| TAGGACG | TAGGACG |
| TCGCTCA | TCGCTCA |
| TCGTAAC | TCGTAAC |
| TGATTAT | TGATTAT |
| TGCACAG | TGCACAG |
| TGGATCG | TGGATCG |

In certain embodiments, the two barcodes used herein (for a particular target insert in a duplex) do not have the same length (and may or may not be complimentary, and may be the same sequence). Thus, combinations of barcode design of different lengths further expands the design space that one may employ in the methods and compositions herein. It is noted that one exemplary advantage of this significantly expanded barcode design space reduces the requirement of barcode length for a same level of adaptor pool complexity. This can reduce the length of the adaptor oligonucleotides, and thereby reduce the synthesis cost. In further embodiments, the associated barcodes may contain additional degenerate bases of equal or unequal length. In addition, the ends of the target insert sequence (endogenous barcodes) can also, or alternatively used, as barcodes in any of the methods herein.

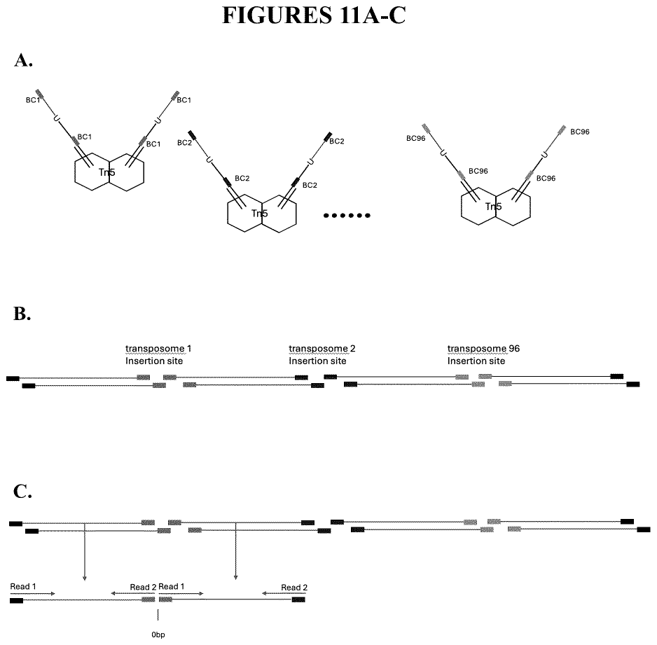

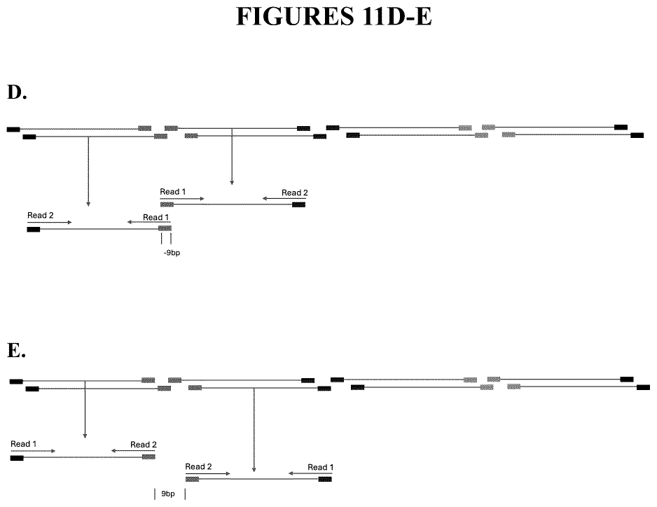

In certain embodiments, transposons with a set of barcodes on the first and second adaptors are loaded onto transposase separately. For example, transposons with 96 sets of barcodes (Table 4) are loaded onto Tn5 separately, forming 96 individual transposome with each having the same barcodes on the adaptor (FIG. 11A). All individually loaded transposomes are then mixed and used for the tagmentation of the duplex molecules.

After transposition, individual transposome targets nucleic acid (e.g., DNA) and generates two duplex ends at the insertion site (FIG. 11B). After the completion of tagmentation and looped ligation and cleavage process, each end of the two duplex ends is attached with a same barcode (FIG. 11B). After sequencing, the genomic coordinates of the ends and the associated barcode information can help to reconstitute the DNA contiguity information.

As shown in FIG. 11C/D/E, amplicons that are derived from dual-tagged strands are sequenced. Among the 4 DNA strands at each insertion site, a minimum of 2 strands (one on each side of the insertion site) should be sequenced to restore the contiguity before the transposome insertion. Read pairs that satisfy: (1) one of the end coordinates of the sequenced strand differ by 0, or 9, or −9 bases (FIG. 11C/D/E); (2) same barcodes are present at the ends in (1). Candidate read pairs can be “concatenated” to form longer reads, extending the contiguity information of the sequenced DNA. For some insertion site, as many as 4 strands can be sequenced. In such cases, not only continuity information, but also duplex information, can be restored simultaneously.

In some embodiments, the barcodes (e.g., non-complementary barcodes; same sequence) may have a length of 1, 2, 3, 4, 5, 6, 7, 8, 9, 10, 11, 12, 13, 14, or more nucleotides. In particular embodiments, the non-complementary barcodes have 1, 2, 3 or more mismatches. In particular embodiments, barcodes that form pairs with maximal mismatches (hamming distance). In other embodiments, the barcodes are exactly the same. In further embodiments, the strand combinations of two barcodes have a length difference, such as 1, 2, 3, 4, or more differences. In particular embodiments, one of the two barcodes in the combination is not present (zero nucleotides). In other the barcodes have certain constant and/or degenerate bases.

In certain embodiments, the ligase employed here is: Circligase I, Circligase II, Mth DNA ligase, RtcB ligase from E. coli., thermostable RtcB, LoopLigase (QIAGEN), Tph ssDNA ligase SDL (see, Miura et al., Nucleic Acids Research, Volume 53, Issue 3, 10 Feb. 2025, herein incorporated by reference), T4 DNA ligase, Hyper-ligase (see Yu Zheng et al., patent EP3430154B1, here incorporated by reference, Ref 14), Taq DNA ligase, T4 RNA ligase 1, and T4 RNA ligase 2. In some embodiments, the ligase is a single stranded ligase, such as a thermostable lysine-mutant such as those provided in SEQ ID Nos:23-43 in Tables 5 and 6 below, or N or C terminal truncated, versions thereof. In some embodiments, when the ligase employed is a single stranded ligase that has step 3 ligase activity, but not step 2 adenylyl transfer activity, the 5′-end of the transferred strand is adenylated to facilitate the subsequent loop ligation.

TABLE 5
Thermostable Lysine-Mutant ssDNA/RNA Ligases

| Amino Acid Sequence | SEQ ID NO: | Species name |
|---|---|---|
| MVSSYFRNLLLKLGLPEERLEVLEGKGALAEDEFEGIRYVRFRDSARNFRRG | 23 | Thermococcus |
| TVVFETGEAVLGFPHIKRVVQLENGIRRVFKNKPFYVEEXVDGYNVRVVKVK | | kodakarensis |
| DKILAITRGGFVCPFTTERIEDFVNFDFFKDYPNLVLVGEMAGPESPYLVEG | | |
| PPYVKEDIEFFLFDIQEKGTGRSLPAEERYRLAEEYGIPQVERFGLYDSSKV | | |
| GELKELIEWLSEEKREGIVMKSPDMRRIAKYVTPYANINDIKIGSHIFFDLP | | |
| HGYFMGRIKRLAFYLAENHVRGEEFENYAKALGTALLRPFVESIHEVANGGE | | |
| VDETFTVRVKNITTAHKMVTHFERLGVKIHIEDIEDLGNGYWRITFKRVYPD | | |
| ATREIRELWNGLAFVD | | |
| Where X is any amino acid except K. | | |
| MVSSHFKEILMRLGLPEDRIEVLEAKGGITEEEFDGIRYLRFKDSARGLRRG | 24 | Pyrococcus yayanosii |
| TVVFDEANVILGFPHIKRVVSLRAGVMRIFKRTPFYVEEXVDGYNVRVALVS | | |
| DRVLAITRGGFVCPFTTERILDFVPEEFFKDYPHLVLVGEMAGPESPYLVEG | | |
| PPYVEEDIRFFLFDIQEKGTGKSLPVQERLKLAEEYGIPHVKVFGLYTVDRI | | |
| EDLYDLIERLSREGREGVVMKSPDMKRVVKYVTPFANVNDVKIGAKVFFELP | | |
| PGYFMSRIMRLAFYVAERRIKGERFEELARNLGKALLEPFVESIWDVEQGDE | | |
| IAEVFRIRVKRIETAYKMVTHFERLGLNIKIEDIEEVGGMWRITFKRAYDEA | | |
| TREIRELIGGRAFVD | | |
| Where X is any amino acid except K. | | |
| MVSSKFKDILYRLGIPEGKVEDLEARGGLVEDKFDDIKYLRIRNSVGKLRRG | 25 | Pyrococcus horikoshii |
| TVVLNDKFIILGFPHIKRIVNLKNGIKRTFKRGEFYVEEXVDGYNVRVVKFR | | |
| GKILGITRGGFICPFTTERISDFIPEEFFKDHPNLILVGEMAGPESPYLVEG | | |
| PPYVKEDIQFFLFDIQELGTGRSLPVEERLKIAEEYGISHVEVFGKFTYKDL | | |
| EEIYEIVERLSREGREGIVMKSPDMRKMVKYVTPYANINDIKIGARVFYELP | | |
| PGYFTSRISRLAFYIAEKRLRGENFEELAKELGKALLQPLVESIHDVEQEDE | | |
| IAEVFKVRVKKIETAYKMVTHFEKLGLRIEIVDIEEMKGGWRITFKRLYPDA | | |
| TEEIRELIGGKSFVD | | |
| Where X is any amino acid except K. | | |
| MKEVVSSVYKEILVKLGLTEDRIETLEMKGGIIEDEFDGIRYVRFKDSAGKL | 26 | Pyrococcus abyssi |
| RRGTVVIDEEYVIPGFPHIKRIINLRSGIRRIFKRGEFYVEEXVDGYNVRVV | | |
| MYKGKMLGITRGGFICPFTTERIPDFVPQEFFKDNPNLILVGEMAGPESPYL | | |
| VEGPPYVKEDIQFFLFDVQEIKTGRSLPVEERLKIAEEYGINHVEVFGKYTK | | |
| DDVDELYQLIERLSKEGREGIIMKSPDMKKIVKYVTPYANINDIKIGARVFY | | |
| ELPPGYFTSRISRLAFYLAEKRIKGEEFERVAKELGSALLQPFVESIFDVEQ | | |
| EEDIHELFKVRVKRIETAYKMVTHFEKLGLKIEIVDIEEIKDGWRITFKRLY | | |
| PDATNEIRELIGGKAFVD | | |
| Where X is any amino acid except K. | | |
| MENMVSSKFKELLYTLGIPEDKVEILEARGGIMEDEFEGIRYLRFKNSVGKL | 27 | Pyrococcus furiosus |
| RRGTVLFEDGTTVFGFPHIKRIVNLSAGVRKIFKSSEFYVEEXVDGYNVRVV | | |
| KFKDRILGITRGGFICPYTTERIAEFVPEEFFKDHKDLVLVGEMAGPESPYL | | |
| VEGPPYVKEDIQFFLFDIQDIKTGSSLPVEERLKLAEEYGINHVEVFGRYSY | | |
| KDIDDLYELIERLSREGREGIVMKSPDMKKIVKYVTPYANINDIKIGARVFY | | |
| ELPGGYFTSRISRLAFYIAEKKIRGEELHNLALQLGKALLQPLVEAIHDVTQ | | |
| GDVIAERFRVRVRKIETAYKMVTHFEKLGLEIEIEDIEEIEGGWRVTFKRVY | | |
| PEATREIRDLIGGKAFVD | | |
| Where X is any amino acid except K. | | |
| MTWIKNPEPWMVNLVAEKLGLDVERVETLARHGTIRFRGYRDVVYALLRREI | 28 | Hyperthermus |
| AGHPEGTVVLLERNGARLVPGYPPIQRMVLPTIALPRHFIDKVVVEEXMNGY | | butylicus |
| NVRLVMFHRKLLAVTRGGFICPYTTARLERLIGGRVRELFREIDPETYTIAG | | |
| EVVGLENPYTRYFYPEAPRFDYFVFDLFHELKPLPPLERNELLEKYGLKHVR | | |
| LLGVIDKNDVEMFKQIVAELDREGREGVVAKDPEYRVPPLKYTTSAVNIGDV | | |
| RYGMRFFMEEGRSFLFSRLLRELFRAYEEGFGDAQLEKLALEFGRAATEPAL | | |
| ESIRKVAMGDMLYEEFELVFADEVELEEFTSYMAELGVDIVVVSTSREDEGL | | |
| RARMRKIKDTWIQLRKVLDTGLSPVD | | |
| Where X is any amino acid except K. | | |
| MTWIKNPEPWMVNLVAEKLGLDVERVETLARHGTIRFRGYRDVVYALLRREI | 29 | HbuRN12K106A |
| AGHPEGTVVLLERNGARLVPGYPPIQRMVLPTIALPRHFIDKVVVEEAMNGY | | |
| NVRLVMFHRKLLAVTRGGFICPYTTARLERLIGGRVRELFREIDPETYTIAG | | |
| EVVGLENPYTRYFYPEAPRFDYFVFDLFHELKPLPPLERNELLEKYGLKHVR | | |
| LLGVIDKNDVEMFKQIVAELDREGREGVVAKDPEYRVPPLKYTTSAVNIGDV | | |
| RYGMRFFMEEGRSFLFSRLLRELFRAYEEGFGDAQLEKLALEFGRAATEPAL | | |
| ESIRKVAMGDMLYEEFELVFADEVELEEFTSYMAELGVDIVVVSTSREDEGL | | |
| RARMRKIKDTWIQLRKVLDTGLSPVD | | |
| MASAAEVLASALRAVGVDPGSVDLEALSTRRSVRVSRFEDVVYVGFRRQFRG | 30 | Aeropyrum pernix |
| VPEGTLVAFRRGEQIVVWGYPSIKRMLLPRVAVPRWFPGPTVLVEEXMNGYN | | |
| VRVFTLGGMVYAATRGGLICPYTTRRLRRLYGGALQKILEDLGAEGSFIAGE | | |
| VVGLENPYTRYYYEEAPGFGYFIFDIFKGGRQLPPRVKFSLAPEYGLKTVNL | | |
| LAEIPATASGVERLYTIVEDLEKRGREGVIVKDPEGRVEPLKYTTSRINIGD | | |
| IRLGMRYPFEEGRSFLFPRILREIFREWETGRRRYGELGEAILAPAIEAVEA | | |
| VSRGGRLVEEFELVFANEVEAEEVIAYFASLGVHLEIAGVARGVDGVRVAFR | | |
| KPRKSEGEIARILETGISPLD | | |
| Where X is any amino acid except K. | | |
| MDENELVNKLSDALGIEYEKLSKHIGRSIRLMKYGELNYVVFRRDLLGYREG | 31 | Staphylothermus |
| TTILLGEEPLIVHGYPSIQRLAFIEGVSKHMIDNVVVEEXMNGYNVRVVYYM | | marinus |
| NNIYAITRGGYICPYTTARIRKLYSKNIKLAYQEYPDTILVGEVVGTENPYV | | |
| IYDYPEARGFDYFIFDTMKKDKLQPLRIRDEIAEKYSLKTVRILDIINKRDI | | |
| DRLKTIINRLEKERREGVVLKDPYQRVPPLKYTTIYINIRDIWEGMRYPFDE | | |
| GRGYLFSRIVRLIAQGYEYDWNNTELDRIALKLGRAILEPAINSLKKRANGE | | |
| IIASKYTLVFPSEDDLSKYIEYAESIGMDFIFRVVEKREDGCIVVELFKMKE | | |
| THNIYTKMLKTGYSPLD | | |
| Where X is any amino acid except K. | | |
| MIRIPLERWMIEKLAEALNVNIEEAERLARRRNVVRLMKWRNVTYFSLRKDV | 32 | Pyrolobus fumarii |
| YGLREGTLIAVWPDGYRVVPGYPSIQRVLLPSVALPKHFIDKIVVEEXLNGY | | |
| NVRVVKLRDEIVAVTRGGLICPYTTQRIRKLYGDKLTSLFREEGEELVVAGE | | |
| VIGLENPYVRFYYPEAGGFAYFIFDIVHGEKFLPPHERKEIVEKHGLLHVPV | | |
| LGEIDKNDIKAFRKIIEDLERRGREGVVLKDPEYRVPPLKYTTSFINIHDIE | | |
| IGMRFPFDEGRNYLFSRILREIFKAVEEGWDDRRLLLAEQNLGKAILEPAIE | | |
| AVKEVKNGKMLYEEFMLPFDTRDDFEEFLDYMASLGVDIIVAGVEQRSDGSI | | |
| VARIRKVKDTWREVQKILETGLSPID | | |
| Where X is any amino acid except K. | | |
| MISPELVKEALKKKKVRSEEAFGLEYLRFNDDYKDIPRGTAIFKDFIIWGYP | 33 | Aquifex aeolicus |
| HIGRIFLLETGLREQFEAPFWVEEXVDGYNTRIFKYGDNYYALSRGGFICPF | | |
| TTDRLPDLIDLRILDENPDLVICAEVAGPENPYIEESPPYVKEDVQLFVFDF | | |
| MKKNEQGFLSQEEKMELIEKYNLPHVEILGRFTASEEGIKKIKEILKRFNEE | | |
| GREGVVFKEDSERNKRAKYITSYANLMDIKTNAKNMLQLPPEYYTNRILRLV | | |
| LFMYEEGLKTTEHLYEELGRAFIDGLFQAIEQFEKEHKVYKTFTCKFRKKEN | | |
| AIALLELLSKTSKHIQVKERRLEKEGDYWRLEFDKVFLNMTGLLGHLLSGGI | | |
| VYD | | |
| Where X is any amino acid except K. | | |

TABLE 6

| Amino Acid Sequence | SEQ ID NO: | Species name |
|---|---|---|
| MTWIHSPESWMLDVVAEALGIDRERVEHLARHRTIRYRVFRGILYASLRREV | 34 | Pyrodictium |
| AGHPEGTVIVFGRGWWRLIPGYPSIQRMVLPSVALPRHFVDKIVVEEXLNGY | | delaneyi |
| NVRVALIDDRIIAVTRGGFICPYTTSRLERIMGNQLKDMLRELGPEEHVAAG | | |
| EVIGLENPYTRYFYPEAPRFGYFVFDVFREGKPLPPGWRDEVTEKHGVPHVP | | |
| VLGVLDKNDIEGFKKIVERLNQEGREGVIVKDPEYRVPPLKYTTPATNIGDI | | |
| RYGMRFFMEEGRGFLFSRLLREIFRVYEEGLIGPRLDALALELGRAALQPAI | | |
| ETVKKVAAGDMVYEEFELEFASRSELEEFMDYMQGLGVDLVLVEIREENGLL | | |
| KTRIRKMKETWLQVRKMLETGLTPID | | |
| Where X is any amino acid except K. | | |
| MRRDVSQFANKLDIGKVSELLDIPEHRITGALKRKTIQYVWGKKELFRFDKP | 35 | Candidatus |
| VSSIEGGTSVFTEPFDIVRGFPKISRTLMLSPALQKHFSSCRKVAVEEXMNG | | Methanoperedens |
| YNVRVALIGDALVALTRGGFICPYTTEKAIDLIGYDFFNDHPDLVLCGEMVG | | |
| PDSPYVPKTFYDIESLDFFVFDIREKITGKPLSVMERRALVDKYGIKSVRLF | | |
| GEFEIGETHSEITRIIKDLGGSQHEGVVIKDPQMVVPPMKYTSSESNCADLR | | |
| YAFEFYNDFGRDFFFGRVCREAFQSVEWDEDEESVEKRCRQLGESLLLPMIK | | |
| TIKKKKDGERIAENVQIRVKSLDTVKEFEEYLKLVGVDAVFEEPEQTGNEYF | | |
| VRIRKMHQSTNDRTEAILGGQLWS | | |
| Where X is any amino acid except K. | | |
| MTWIHRPEPWMLDVVADALGLPRERVEELASRRTLRFREFRGLLYASLRRGV | 36 | Pyrodictium occultum |
| AGHHEGTAVVFGRGWWRVVPGYPPIQRMVLPSVALPRHFLDRVVVEEXLNGY | | |
| NVRVVLVDDRILAVTRGGLICPYTTSRLERLMGDRLREMLRELGPEDHVAAG | | |
| EVIGLENPYTRYFYPEAPRFGYFVFDIFRGGRPLPPRMRDEAAEKHGVPHVP | | |
| VLGVLEKTDVEAFKRIVERLDREGREGVVVKDPDYRVPPLKYTTSSTNIGDI | | |
| RLGMRFFMEEGWSFLFSRILREIFRVYEEGVEGPRLDAIALELGRAALQPAV | | |
| ETVKKVAGGYMVYEEFELEFAGRDELEEFMDYMQSLGVDVVLVEAREEGGVL | | |
| RARMRKIKETWIRVRRILETGVSPID | | |
| Where X is any amino acid except K. | | |
| MGWVQPEPWMVDAVAEALGLERERVESLAKHRTIRFRVFRGILYASLRRELG | 37 | Thermoproteota |
| GYPEGTVVIFGRGWSRVVHGYPPIQRMVLPSVALPRHFVDRIVVEEXLNGYN | | archaeon |
| VRVVLVDGRLLAVTRGGFICPYTTDRIERLLGGRLREMLRELGEEEHVAAGE | | |
| VIGLENPYTRYYYPEAPRFGYFVFDIFRSGKPLPPRVRDEATEKHGVPHVPV | | |
| LGVLDKGDIEGFRSIVEALERRGREGVVVKDPEYRVHPLKYTTHATNVGDIR | | |
| LGMRFFMEEGRGFLFSRLLREIFRAYEQGLQGPRLEKLATEIGLAALEPALE | | |
| TVRLVAAGEPVYEEFELEFENRDRLEEFLEYMQSLGVDVVVAGTYERDGMLV | | |
| ARVRKMRDTWLQVRRMLETGLTPID | | |
| Where X is any amino acid except K. | | |
| MFVSESLGLSKHLGETLEERKILREALISHSFFSDVIEAVRFDKKFGEIEEG | 38 | Geoglobus |
| TVVAKTINGVRIVRGFPKIKRALVLNPTLKKHFENEVAVEEXMNGYNVRIAR | | acetivorans |
| FGKNLYAMTRRGIICPYTTEKARELINPEFFKDHSDLVLCCEAVGEESPYVP | | |
| KSMYGVEGLDFFVFDIREERTNRPLPVEEKLRLCEEYGLRHATYFGTYDVDV | | |
| AHDEIKDIISDLAGKGREGVVIKDPEMKLSPLKYTTSQTNAEDLKYAFRFFN | | |
| DYGKDFMFSRIVREGFQSFEFNEGDKEFRERCLRLGMAILKPMVESIREVAL | | |
| GGKVSEKLRLRFGSLDVMNLFFEQWKRSKVDFEITDIKKDGKDIVVFVNKTM | | |
| RNTTDKIKAHLEGIPW | | |
| Where X is any amino acid except K. | | |
| MKFIAEALGVSQAVIEKLNEKNLIRLAFIKHPFFRDVIEAYKLERKVGEFEP | 39 | Archaeoglobus |
| GTLIAKTVEGLRVVRGYPKIKRALTLYPTIKKHFKGEVVLEEXMNGYNVRLV | | profundus |
| KFGENIYAITRGGFICPYTTEKARRLVNLDFFKDNPKLMLCCEAVGEESPFV | | |
| PKDVYGVKTIDFYVFDIRDQKTNIALPIKQKEKLAEEYGLKLAPILAEVQVS | | |
| KAHEIAKEIILELDKRGREGIVIKDPMMRRPPIKYTTSQCNCSDLSYAFRFF | | |
| EEYGKDFMFSRIIREAFQSFEFRENEEKFKDRCLRLGEAILSMVKSIKEVNE | | |
| GKRIVEKMRLRFYDLEIFELFKEHIRRMGIRAEFSNPKREEDGYVVWVYRHI | | |
| MSTTDKIKYILAGNLY | | |
| Where X is any amino acid except K. | | |
| MVSSHFKSLLLELGISRERIEILESKGGIVEDEFEGIRYLRFKDSAGSLRRG | 40 | Thermococcus |
| TVVFDSHNIILGFPHIKRVVHLENGIKRVFKRKPFYVEEXVDGYNIRVAQIE | | litoralis |
| GRVFAFTRGGFVCPFTTERIEDFVNMEFFKDYPNLVLCGEMAGPESPYLVEG | | |
| PPYVKEDIEFFLFDIQEKKTGKSLIVEERLKIAEEYGIPSVEVFGVYDISKI | | |
| DELKELIEQLSREKREGIVMKSPDMKKIVKYVTPYANVNDIKIGARIFFDLP | | |
| HGYFMQRIKRLAFYLAEKRVQDEEFEKYARALGRALLEPFVESIWDVSAGEE | | |
| IAEVFTVRVKHIETAYKMVSHFERLGLKIHIEDIEEMPQGYWRITFKRVYPD | | |
| ATREIRELWSGHAFVD | | |
| Where X is any amino acid except K. | | |
| MVSSRFKDILTSLGISEERIEILEAKGGIVEDEYEGLRYLRFKDSAGKLRRG | 41 | Pyrococcus sp. |
| TVVFDFDKIILGFPHIKRVVNLEKGIRRIFKRGEFYVEEXVDGYNVRVTKVG | | ST04 |
| ERILAITRGGFICPFTTERITDFVPEEFFKDNPNLVLVGEMAGPESPYLVEG | | |
| PPYVKEDIKFFLFDVQEINTGKSLPVEERLKLAEEYGIPHVEVFGKYTRDDI | | |
| GELYALIEKLSEEGREGIVMKSPDMKKIVKYVTPYANINDIKIGARVFYELP | | |
| PGYFTSRISRLAFYIAERKIRDEELRKLAEDLGKALLQPFVEGILDVEQGEE | | |
| IAETFKIRVKKIETAYKMVTHFEKLGLNIEIVDIEEMDGLWRITFKRVYSDA | | |
| TEKIKELVGGKAFVD | | |
| Where X is any amino acid except K. | | |
| MKSERGIMKYKDFIYYPFKKGGFGKGSVIIYHNDDVKIVPGYPSIKRLVLLS | 42 | Ignicoccus pacificus |
| KVPEHFPEGVSVEEXMNGYNVRAMIVGGDVAFITRGGYLCPYTNARLNTLYG | | DSM 13166 |
| EKVKALLEELPPGSFLAGEVVGVENPYVRVKYPEAPYFDYFIFDIFVKTEDG | | |
| WRQMPVEERHEIVKRHGLRSVRLLGTFESSEAPLKIKEIIDRFDKEGREGVV | | |
| MKDPEYKRSPAKYTGSYTNIGDIREGMRYPFDEGKDYLFPRIVREIFKVYEE | | |
| GLSDKELERRALELGMAILKPAVESLKEVAQGETLFERFVLRFPHEEDLEEY | | |
| LNYTRSLGVKVIVEEKWEEGEWIVVKAKKFKNTSNVYRSMLKSGQTPLD | | |
| Where X is any amino acid except K. | | |
| MVSSYFKGILLNLGLDEERIEVLENKGGIVEDEFEGMRYLRLKDSARSLRRG | 43 | Palaeococcus |
| TVVFDEHNIILGFPHIKRVVQLENGIRRAFKRKPFYVEEXVDGYNVRVAKIG | | pacificus |
| EKILVFTRGGFVCPFTTERIEDFITLDFFKDYPNMVLCGEMAGPESPYLVEG | | |
| PPYVKEDIQFFLFDIQEKKTGRSLPVEERLKLAEEYGIPSVEVFGLYDLSRI | | |
| DELHALIDRLTKEKREGIVMKSPDMKKIVKYVTPYANINDIKIGARIFFDLP | | |
| HGYFMQRIKRLAFYLAERKIRGEEFDEYARALGKVLLEPFVESIWDISSGDD | | |
| EIAELFTVRVKKLETAHKMVTHFERLRLKIHIDDIEVLDNGYWRITFKRVYP | | |
| DATKEMRELWNGHAFVD | | |
| Where X is any amino acid except K. | | |

In some embodiments, the sequences in Table 5 or 6 above are used to perform a sequence search (e.g., using BLAST or PSI-BLAST) to find other thermostable ssDNA/RNA ligases from other species (e.g., by finding those with 30% . . . 0.40% . . . 50% . . . 60% or more homology). For a particular candidate homolog that is identified, the next step is to find out the growth temperature of the species it is from. In general, a useful single strand ligase candidate would come from a species that has a growth temperature range higher than about 65° C. Next, one can perform a multiple sequence alignment, and locate the conserved catalytic motif, EKxxG (x is any amino acid; such as shown in Tables 5 and 6 above). Next, within the catalytic motif, mutate K to any other amino acid (e.g., to make a step 3 ligase mutant). In certain embodiments, the lysine (K) in such Motif I is mutated to another amino acid, preferably an alanine (A), serine (S), cysteine (C), valine (V), threonine (T), and Glycine (G). Such candidate enzymes (e.g., mutant enzymes) can then be screened for ssDNA and ssRNA activities (and thermostability).

FIG. 10 also shows how 5′ and 3′ protruding ends (e.g., generated by a transposase) can be preserved by the methods and compositions herein. Current end repair/A-tailing (ER/AT) or polymerase extension during NGS library preparation results in “writing” and “erasing” activities to the starting material, which abolishes the DNA ends (created by a transposase). Provided herein, in certain embodiments, is a sequencing library preparation workflow that preserve such ends, which may have methylation or other information important to determine in sequence reads.

As used herein, a “transposase” means an enzyme that is capable of forming a functional complex with a transposon containing composition (e.g., transposons, transposon ends, transposon end compositions) and catalyzing insertion or transposition of the transposon end-containing composition into a double-stranded target nucleic acid. Exemplary transposases that can be used with certain embodiments provided herein include (or are encoded by): Tn5 transposase, Sleeping Beauty (SB) transposase, Vibrio harveyi, MuA transposase and a Mu transposase recognition site comprising R1 and R2 end sequences, Staphylococcus aureus Tn552, Ty1, Tn7 transposase, Tn/O and IS10, Mariner transposase, Tc1, P Element, Tn3, bacterial insertion sequences, retroviruses, and retrotransposon of yeast. Additional examples include IS5, Tn10, Tn903, IS911, and engineered versions of transposase family enzymes.

In some embodiments, the transposase is a Tn5, Tn7, MuA, or Vibrio harveyi transposase, or an active mutant thereof. In other embodiments, the transposase is a Tn5 transposase or a mutant thereof. In some embodiments, the Tn5 transposase is a hyperactive Tn5 transposase, or an active mutant thereof. In some embodiments, the Tn5 transposase is a Tn5 transposase as described in U.S. Pat. No. 9,790,476 which is incorporated herein by reference. In some embodiments, the Tn5 transposase is a hyperactive Tn5 with mutations at positions 54, 56, 372, 212, 214, 251, and 338 relative to wild-type Tn5 transposase. In certain embodiments, the Tn5 transposase is a hyperactive Tn5 with the following mutations relative to wild-type Tn5 transposase: E54K, M56A, L372P, K212R, P214R, G251R, and A338V. In some embodiments, the Tn5 transposase is a fusion protein. In some embodiments, the Tn5 transposase fusion protein comprises a fused elongation factor Ts (Tsf) tag. In particular embodiments, the Tn5 transposase is a hyperactive Tn5 transposase comprising mutations at amino acids 54, 56, and 372 relative to the wild type sequence. In some embodiments, the recognition site is a Tn5-type transposase recognition site (Goryshin and Reznikoff, J. Biol. Chem., 273:7367, 1998). In particular embodiments, a transposase recognition site that forms a complex with a hyperactive Tn5 transposase is used (e.g., EZ-Tn5™ Transposase, Epicentre Biotechnologies, Madison, Wis.). In some embodiments, the Tn5 transposase is an engineered mutant with less insertion bias, such as TnX (SeqWell). In some embodiments, the Tn5 transposase is a wild-type Tn5 transposase.

In some embodiments, the transposome complexes herein comprise a dimer of two molecules of a transposase. In some embodiments, the transposome complex is a homodimer, wherein two molecules of a transposase are each bound to first and second transposons of the same type (e.g., the sequences of the two transposons bound to each monomer are the same, forming a “homodimer”). In some embodiments, the compositions and methods described herein employ two or more populations of transposome complexes. In some embodiments, the transposases in each population are the same. In some embodiments, the transposome complexes in each population are homodimers, wherein the first population has a first cleavable region in each monomer and the second population has a different cleavable region in each monomer. In some embodiments, the transposome complexes within each population has the same set of barcodes in transposon strands, but different from each population.

In certain embodiments, the partially double stranded transposon complexes herein have a double-stranded transposon end. As non-limiting examples, transposon ends can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US 2010/0120098, the content of which is incorporated herein by reference in its entirety. Transposon ends can comprise any nucleic acid or nucleic acid analogue suitable for forming a functional complex with the transposase in an in vitro transposition reaction. For example, the transposon ends can comprise DNA, RNA, modified bases, non-natural bases, modified backbone, and can comprise nicks in one or both strands. Although the term “DNA” is used throughout the present disclosure in connection with the composition of transposon ends, it should be understood that any suitable nucleic acid or nucleic acid analogue can be utilized in a transposon end.

In certain embodiments, the transposon complexes herein are used in library preparation (“library prep”) and/or then in sequencing methods, such as in attaching adaptors to library fragments for subsequent sequencing. For example, in some embodiments, the disclosure provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), sequence-by-binding, sequencing-by-expansion (SBX), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

Any number of DNA sequencing techniques are suitable, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the present disclosure finds use in automated sequencing techniques understood in that art. In some embodiments, the present technology finds use in parallel sequencing of partitioned amplicons (PCT Publication No: WO2006084132, herein incorporated by reference in its entirety). In some embodiments, the technology finds use in DNA sequencing by parallel oligonucleotide extension (See, e.g., U.S. Pat. Nos. 5,750,341, and 6,306,597, both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; all of which are herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; all of which are herein incorporated by reference in their entireties), and the Adessi PCR colony technology (Adessi et al. (2000). Nucleic Acid Res. 28, E87; WO 00018957; herein incorporated by reference in its entirety). In certain embodiments, the library preparation and sequencing technologies are as described in any of the following U.S. patents, each of which is herein incorporated by reference: 9,752,188; 10,570,451; 11,479,807; 8,383,345; 10,876,172; 9,598,731; 9,902,992; 10,801,063; 11,091,797; 8,532,930; 9,639,657; and 10,011,870.

In certain embodiments, the 5′ or 3′ end of the transposon strands herein have a 3′ end or 5′ end blocking group such as a phosphate group or a modified noncanonical nucleotide. In certain embodiments, the 5′ end of the first transposon strand is un-phosphorylated, thus rendering it unsuitable for ligation. Before looped ligation, a phosphorylation step (i.e., “deblocking”) is needed to restore it to suitable state. In certain embodiments, the 3′-end of the second transposon strand is blocked (e.g., by C3, C9, C18 spacer), which blocks the 3′-end from participating undesired ligation. If the blocking group is, for example, a nucleotide with 3′-phosphate, such blocking group can be removed by T4 polynucleotide kinase, calf intestinal alkaline phosphatase (CIP), or shrimp alkaline phosphatase (SAP). In certain embodiments, where CIP or SAP are used, one may employ a round of T4 PNK to re-phosphorylate the 5′-end of the proximal strand. Another class of the base blockers and deblocking can leverage reversible terminator technologies widely used in the sequencing industry.

In certain embodiments, the transposons herein comprises a non-canonical base in the cleavable region, such as inosine, uracil, deoxyuridine, 5-formylcytosine, 5-carboxylcytosine, or 8-oxoguanine. In some embodiments, wherein inosine is present in the cleavable region, Endonuclease V or similar enzyme is used to perform strand cleavage. In certain embodiments, wherein uracil, 5-formylcytosine, or 5-carboxylcytosine is present in the cleavage region, a combination of uracil DNA glycosylase (UDG) and Endonuclease VIII (or similar enzymes, such as endonuclease IV) are employed for strand cleavage. In particular embodiments, where 8-oxoguanine is present in the cleavable region, a thermostable OGG (oxoguanine glycosylase) is employed for strand cleavage. In particular embodiment, a cleavable backbone linkage can be, for example, phosphorothioate DNA and cleaved by iodine (I₂) (Qiang Huang et al. Origin of iodine preferential attack at sulfur in phosphorothioate and subsequent P—O or P—S bond dissociation, PNAS, vol 119, 2022).

In particular embodiments, the cleavable region is cut with an endonuclease. For example, a secondary oligo is added to the sample and hybridizes to an adaptor sequence and creates a cleavage site in the cleavable region (e.g., which can be cleaved by a restriction endonuclease). In some embodiments, while most of the adaptor is composed of DNA, the cleavage region contains one or more RNA bases, allowing cleavage by an Rnase H, Rnase H2, or site-specific endonuclease.

In certain embodiments, the first and/or second adapter sequences is/are methylated at every (or almost every) cytosine present. In certain embodiments, the methylation is 5-methylcytosine (5mC) and/or 5-hydroxylmethylcytosine (5hmC). Such methylation, for example, can be introduced during synthesis of the nucleic acid sequences. During bisulfite conversion (e.g., as part of bisulfite sequencing methods herein) 5′-methylcytosine (or 5hmC) will not be converted during the conversion step while unmodified cytosine will be converted to uracil. The conversion can either be chemical based (such as bisulfite conversion) or enzyme based (such as EM-seq). Methylation of the nucleic acid sequences herein prevents, for example, any elements present in the adapters (such as flow cell binding sequences and universal primer binding sites) from having their sequences changed during bisulfite conversion (or other conversion). In this regard, the adapter sequences can still bind to universal primers employed and can still bind to flow cell sequences (e.g., such as in Illumina sequencers).

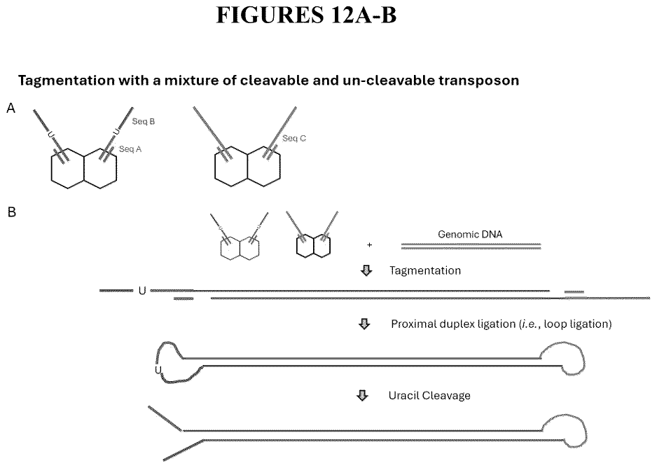

In some embodiments, the library preparation generates targets such as those shown in FIG. 12, which have a closed loop (hairpin) at one end and an two part open adapter at the other end. Such targets (which can be called ‘branched-end hairpin-tethered duplex molecules’) allow, for example, one to reliably obtain genetic and epigenetic sequence data of the entire double stranded template molecule. Often times, traditional duplex sequencing suffers the limitation of low duplex recovery efficiency, in that there is no guarantee that both strands are sequenced from the library as each strand is sequenced separately. Thus, it provides advantage that two strands of the duplex template molecule can be tethered and sequenced together (e.g., as shown in FIG. 12). This is provided, for example, in the CODEC in which one parent strand is directly connected to the daughter of the other parent strand (Bae et al., Nature Genetics, 55(5), 871-879, 2023, herein incorporated by reference). In addition, Roche SBX (sequencing by expansion) sequencing provides the capability of directly sequencing the duplex template by connecting the strands using a stem-loop adaptor (WO2025132779A2, herein incorporated by reference in its entirety). However, in both methods, native duplex template goes through the end repair/A-tailing process (ER/AT) before the oligonucleotide adaptor is ligated. As stated above, the inclusion of ER/AT introduces a variety of artifacts and abolishes the native duplex end information.

In some embodiments, provided herein methods and compositions for preparing duplex molecules that are tethered together at one end using a non-cleavable transposon (e.g., so the top and bottom strands stay together and can be sequenced as a single unit, thereby allowing, for example, top and bottom stranded comparison for correction of errors or artifacts, as well as determination of native duplex ends). In certain embodiments, the methods and compositions herein allow for the preparation of a branched-end hairpin-tethered duplex molecule, such as pictured in FIG. 12, where the duplex strands are tethered together at one end by using a transposon with non-cleavable transferred strand, and have a branched (general V shape) two part adaptor at the other end by using a transposon with cleavable transferred strand. Such branched-end hairpin-tethered duplex molecules can be generated without the need for ER/AT, thus avoiding the risk of introducing ER/AT-associated artifacts and maximizing native information preservation (e.g., fragmentomic end information).

Such branched hairpin library molecules can be sequenced by any suitable methodology, including using sequencing-by-expansion technology commercialized by Roche sequencing solutions, where both strands can be sequenced together in a single read (Kokoris et al., bioRxiv 2025.02.19.639056, herein incorporated by reference).

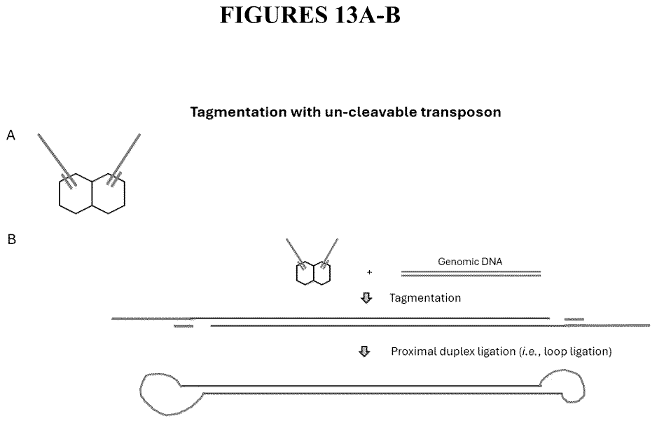

In some embodiments, the library preparation generates targets such as those shown in FIG. 13, which have a closed loop (hairpin) at both ends of the duplex target, by using transposons with non-cleavable transferred strand. Such targets (which can be called dumbbells or similar), can be sequenced. For example, in some embodiments, single-molecule real-time (SMRT) sequencing, as developed and commercialized by Pacific Biosciences of California, Inc. (PacBio), is employed as it allows direct sequencing of individual DNA molecules without the need for clonal amplification, as described, for example, in: U.S. Pat. Nos. 9,910,956 and 11,091,794, which are incorporated herein by reference in their entireties. In PacBio's SMRT sequencing platform, sequencing libraries are prepared by ligating hairpin adapters to both ends of a double-stranded target nucleic acid molecule after ER/AT, thereby forming a dumbbell template referred to by PacBio as a SMRTbell™. Because ER/AT is used, it has the inherent limitation of introducing ER/AT-associated artifacts to sequencing results. The embodiment in FIG. 13 does not involve any ER/AT, thus eliminating these potential artifacts from the root.

### EXAMPLES

### Example 1

### Tagmentation and Looped Ligation Library Preparation

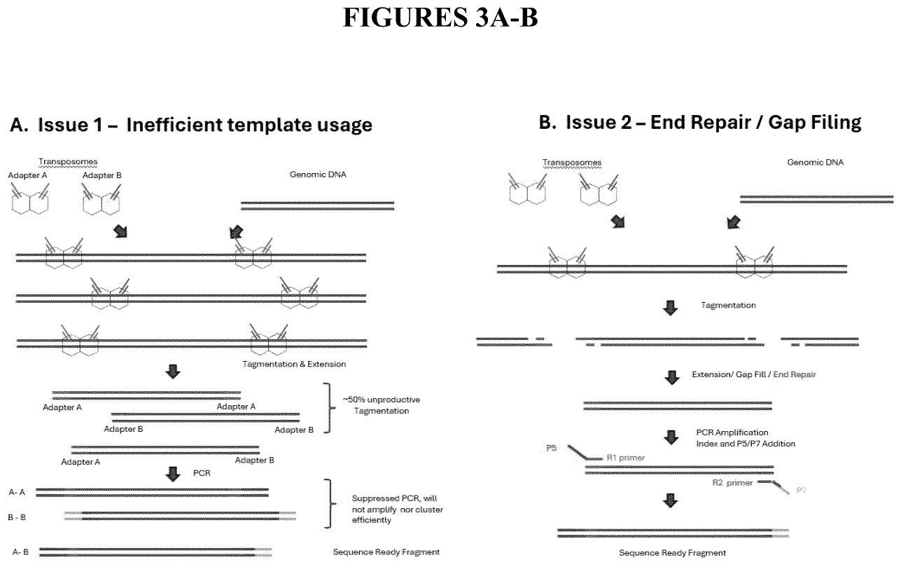

In this Example, a library preparation approach is illustrated called Tagmentation And Looped Ligation (TagALL). In this example, the NGS library preparation method utilizes a 5′ tagmentation followed by direct 3′ proximal looped ligation. This method addresses the problems shown in FIG. 3, such as inefficient template usage (FIG. 3A) and polymerase extension and gap filling that can introduce errors and erase methylation signals that may be present (FIG. 3B). In this regard, this method provides improved library preparation that generates high diversity libraries, retains native methylation signal and tagged sequence ends and displays a reduced error rate.

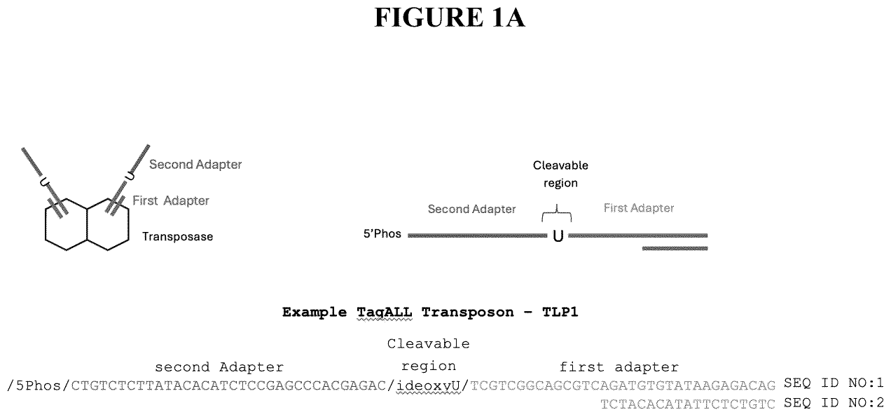

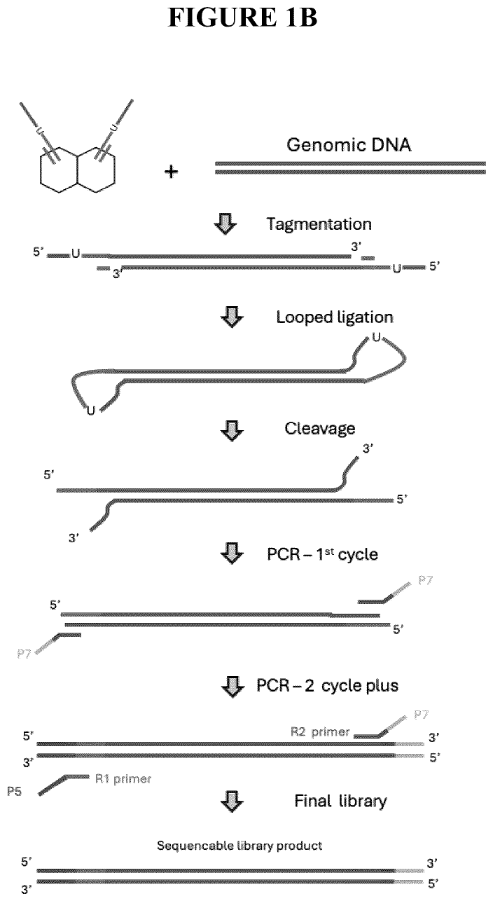

### Results of FIG. 1 Type Methods

### TagALL Library Construction

NGS libraries were prepared following a workflow outlined in FIG. 1, utilizing a novel Transposon sequence that attaches an adapter initially to the 5′ end of the template molecule, and then subsequently via a proximal intra-molecular looped ligation adds the second adapter sequence to the 3′ end of the tagmented molecule. The novel transposon contains both adapter A and adapter B sequences in the same oligo. In this case adapter A and adapter B are Read 1 SP (sequencing primer) and Read 2 SP sequences in an inverted orientation, separated by a cleavable Uracil base. After the initial adapter addition via tagmentation to the 5′ end of the template molecule, the attached adapter's 5′ end is subsequently ligated to the proximal available 3′ end of the complementary strand.

Due to the proximity of the molecule ends, this ligation event is highly efficient and results in a closed looped structure. The closed loops are subsequently linearized by a combination of Uracil-DNA-glycosylase and endonuclease VIII digest (or endonuclease IV) at the uracil base within the adapter. The resulting molecules represent a population with adapter A (read 1 SP) sequence at 5′ end of the template molecule and adapter B (read 2 SP) sequence at 3′ end of template molecules.

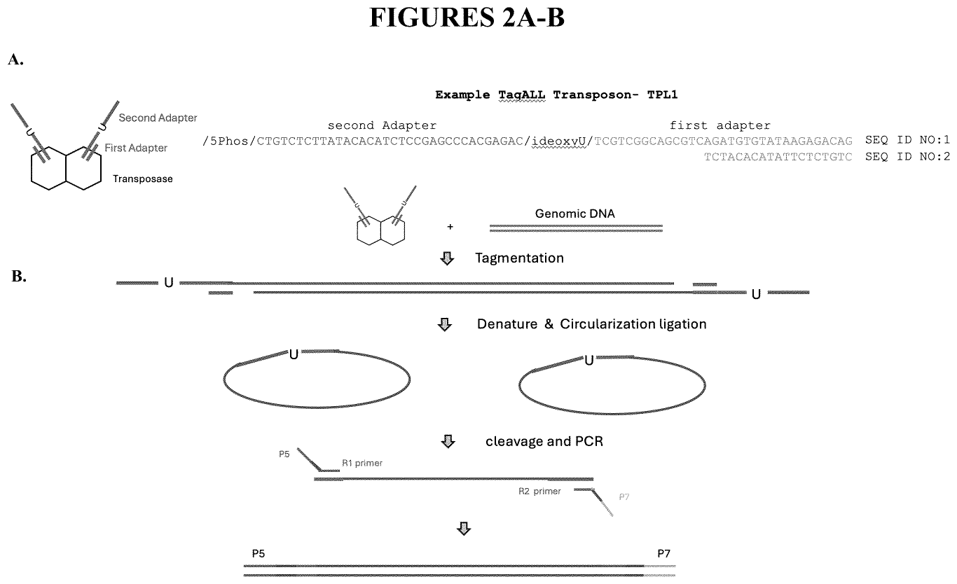

In a second embodiment of the protocol, post tagmentation of TagALL adapter to 5′ end of template, we introduced a template denaturation step, followed by a single strand intra-molecular ligation event (FIG. 2). The molecule is single stranded the opposite 3′ end of the same strand. Due to the proximity of the molecules end this ligation event is efficient and results in a high diversity library product.

### Transposon Adapter Function Design

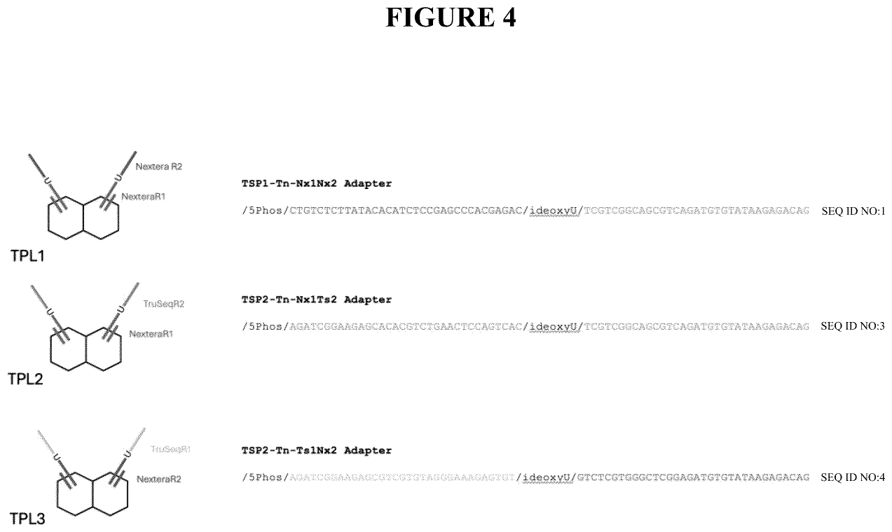

The TagALL Transposon adapters in this example contain several design elements to enable functional library construction (FIG. 1). Libraries are generated from a single adapter, which contains both Read 1 and Read 2 sequencing elements, oriented in opposite directions and includes a cleavable uracil base between the two read sequence elements. After cleavage, one side of the uracil base is attached to the template DNA 5′ end via tagmentation, and the other side of the uracil base is attached to the 3′ end via proximal looped ligation. The nucleotide sequences of the read 1 and read 2 sequencing elements can vary based on user's design, but in the iterations presented in this Example there are three different TagALL Transposon adapters (TSP1, TSP2, TSP3; see FIG. 4). The adapters utilized different combinations of Read 1 and Read 2 core sequences, containing combinations of Illumina Nextera and TruSeq sequencing primer sequences. In order to use a Tn5 Transposase (as in this Example) for tagmentation, the 3′ end of the adapter sequence can use a Nextera compatible read sequence (sometime known as Mosaic End sequence), however the sequence at the 5′ end of the adaptor can be composed of any alternative sequence (e.g., as long a compatible sequencing primer is used in the sequencing process). In the examples shown in this data set, we used Illumina Nextera Read 1 at the 3′ end in combination with Nextera Read 2 (TPL1 design) or TruSeq Read 2 at the 5′ end (TPL2 design), and separately we used Nextera Read 2 at the 3′ end in combination with TruSeq Read 1 at the 5′ end (TPL3 design). All of these read sequences have compatible sequencing primers present in standard Illumina sequencing kits. Alternative read sequences combinations and adapters could be used in conjugation with the use of custom sequencing primers.

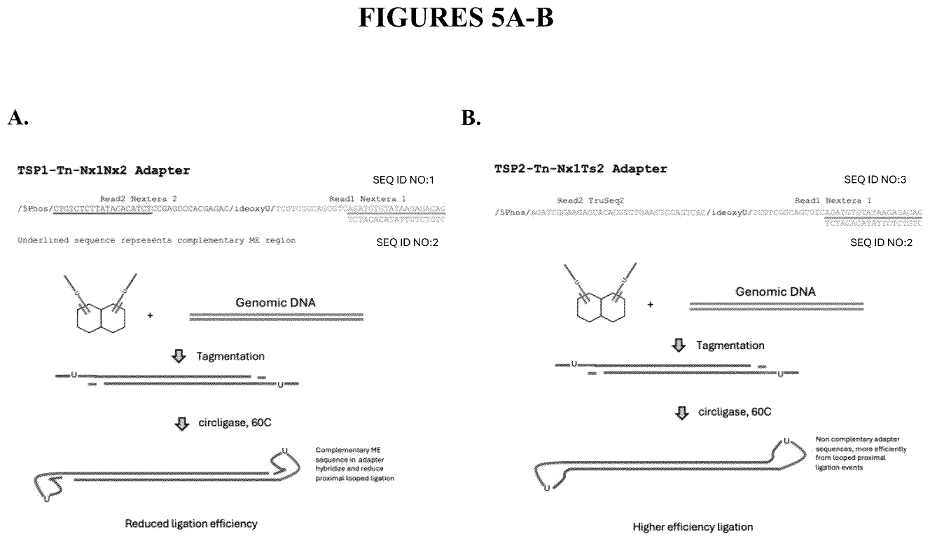

An advantage of using one Nextera read sequence in combination with a TruSeq read sequence or other custom read sequences is that it would reduce interactions of the complementary ME region of the Mosaic End Nextera read sequences (see FIG. 5A). Use of non-complementary read sequences in this context could improve efficiency of the proximal looped ligation reaction, as the presence complementary sequences in adapter form a hairpin structure, effecting the proximal looped ligation by limiting the interactions of the adapter 5′ end and the 3′ template end (see FIG. 5B).

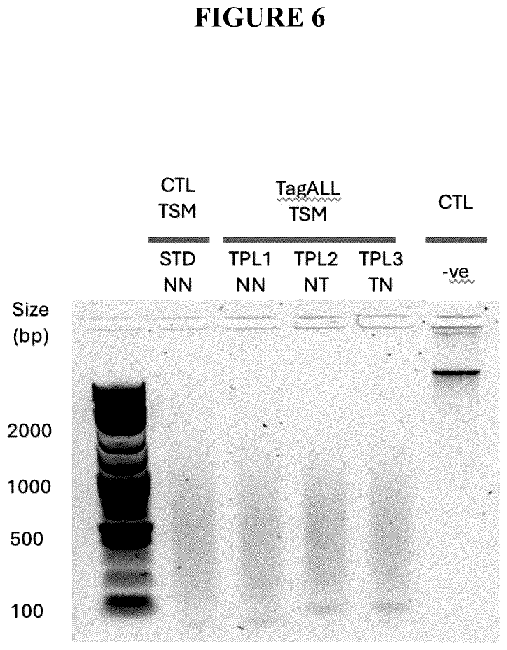

FIG. 6 shows results of some Tagmentation reactions of Human genomic DNA, using several example TagALL Transposome and a control standard Transposon Tagmentation reaction. CTL TSM represents industry standard Transposome with standard transposon sequences. TPL1, TPL2 and TPL3 (see FIG. 4) represent 3 TagALL transposons sequences with alternative A and B sequence combinations (see Table 8 for all Transposon oligo sequences). Tagmentation were carried out with 200 ng of genomic template DNA, and between 12 and 24 pmol of assembled Transposome. Approximately 50 ng of tagmented DNA was loaded on a 0.8% Agarose gel and visualized via SybrSafe staining. All tagmentation reactions show DNA fragmentation profiles of approximately 150-700 bp.

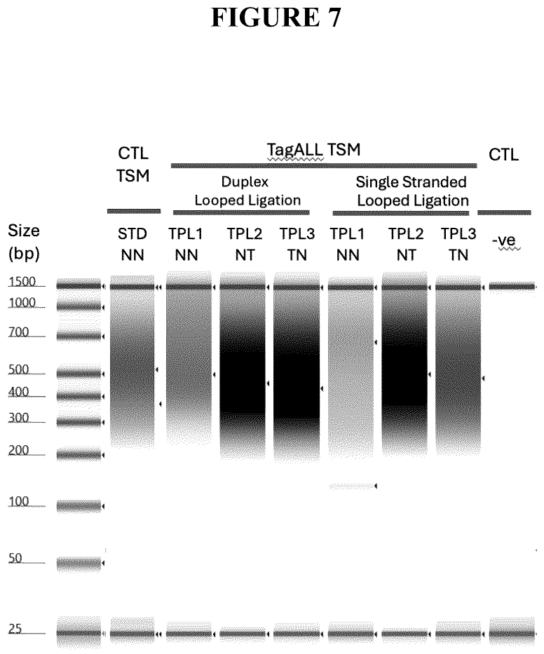

FIG. 7 shows an Agilent High Sensitivity Tapestation analysis of the final amplified libraries prior to sequencing. Control Transposome based libraries representing industry standard library we generated following the protocol described in methods. TagALL Transposome-based libraries were generated from 3 independent Transposome (TSP1, TSP2 & TSP3; FIG. 4). Sample processing was either carried out on the double stranded molecules following the Proximal duplex ligation work flow (FIG. 1) or the Proximal single stranded workflow (FIG. 2). Following proximal ligation all libraries including the standard Transposome Tagmented DNA that did not go through the proximal ligation workflow were all PCR amplified with 12 cycles of PCR using KAPA HiFi hotstart polymerase.

### Results of FIG. 2 Type Methods

### Library Diversity

In order to make all libraries comparable, sequencing data sets were down-sampled to 40 Million read per library and compared for number and percentage or unique and mapped results (Table 7). A high mapping percentage and a high number of unique reads is indicative of a high diversity library preparation. All TagALL libraries had a high number of unique reads, with some TagALL libraries showing higher unique read percentage than control standard Transposon-based library prep method.

TABLE 7

| | Total | | | | |
|---|---|---|---|---|---|
| | input | Unique | % unique | Mapped | |
| Sample | reads | Reads | reads | Reads | % Mapped |
| TSM-STD-NN-DS | 40M | 39M | 98.3% | 40M | 99.5% |
| TSM-TPL1-NN-DS | 40M | 38M | 95.5% | 36M | 89.0% |
| TSM-TPL2-NT-DS | 40M | 40M | 98.9% | 40M | 98.8% |
| TSM-TPL3-TN-DS | 40M | 39M | 98.5% | 40M | 98.9% |
| TSM-TPL1-NN-SS | 40M | 39M | 98.2% | 38M | 95.6% |
| TSM-TPL2-NT-SS | 40M | 40M | 98.8% | 40M | 98.9% |
| TSM-TPL3-TN-SS | 40M | 40M | 98.9% | 39M | 97.5% |

Table 9—Library read mapping QC metrics for 7 NGS libraries with matching input material, 40 Million reads per library. Metrics illustrate that TSM-TPL (TagALL) libraries generated from multiple transposon assemblies with both the single (with SS as suffix) and double stranded protocol (with DS as suffix), generated a comparable number of mapped unique reads to the standard transposon based library (TSM-STD-NN-DS). Some TSM-TPL libraries (e.g., TSM-TPL2-NT-DS, TSM-TPL3-TN-DS) had higher number of unique molecules, indicating that TagALL method is efficient at recovering template and capable of generating highly diverse libraries.

### Evidence of Successful Tagmentation and Looped Ligations

Transposon-based libraries are known to have a slight sequence based insertional bias, which will cause characteristic base composition pattern at the template end (i.e. the insertion site). The polymerase extension step used in standard transposon-based NGS library preparation gap fills the transposition site at the 3′ end, making it complementary to the 5′ insertion site. The consequence of this is that same insertional bias and base composition is present at both ends (i.e. Read 1 and Read 2 of paired end sequencing of an insert) to a library. In TagALL libraries preparation, because the workflow lacks this polymerase gap-filling and instead carries out a looped ligation directly to the tagmented 3′ end of the template, the base composition is expected to be different in Read 1 or Read 2 depending on the composition of the TagALL transposon.

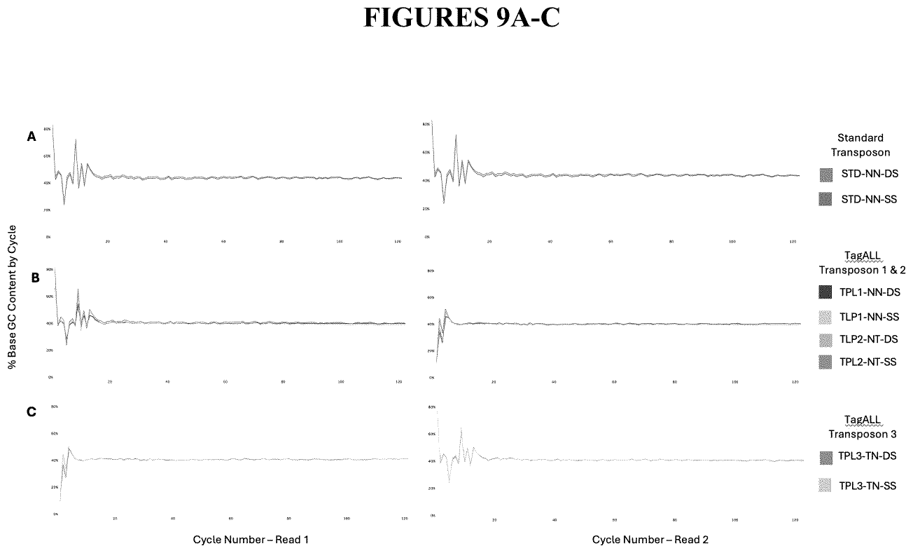

FIG. 9 shows based composition from standard libraries and from two different TagALL compositions. The differing base composition at the start of R1 and R2 demonstrates that a looped ligation event is successfully being completed. In FIG. 9, GC content by read cycle for Read 1 and Read 2 for distinct transposon groups is shown. Illustrating match base-composition on predicted tagmented molecule ends, and a distinct base-composition at looped ligation molecule ends. This evidence indicates successful tagmentation and subsequent ligation events from the TagALL transposons. FIG. 9A shows standard Transposon-based library prep. FIG. 9B shows TagALL TPL1 & TPL2. Read 1 originates from 5′ tagmentation sites and Read 2 from 3′ looped ligation. FIG. 9 C shows TagALL TPL3—Read 1 originates from 3′ looped ligation, and Read 2 originates from 5′ tagmentation fragment end.

### Example of Duplex Identification Using Pre-Defined UMI Pairing

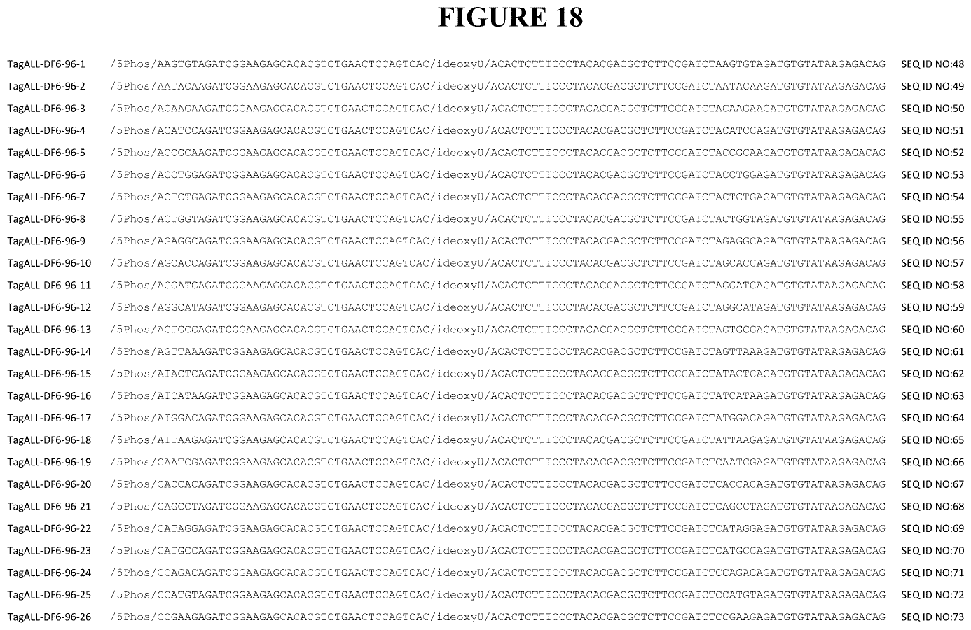

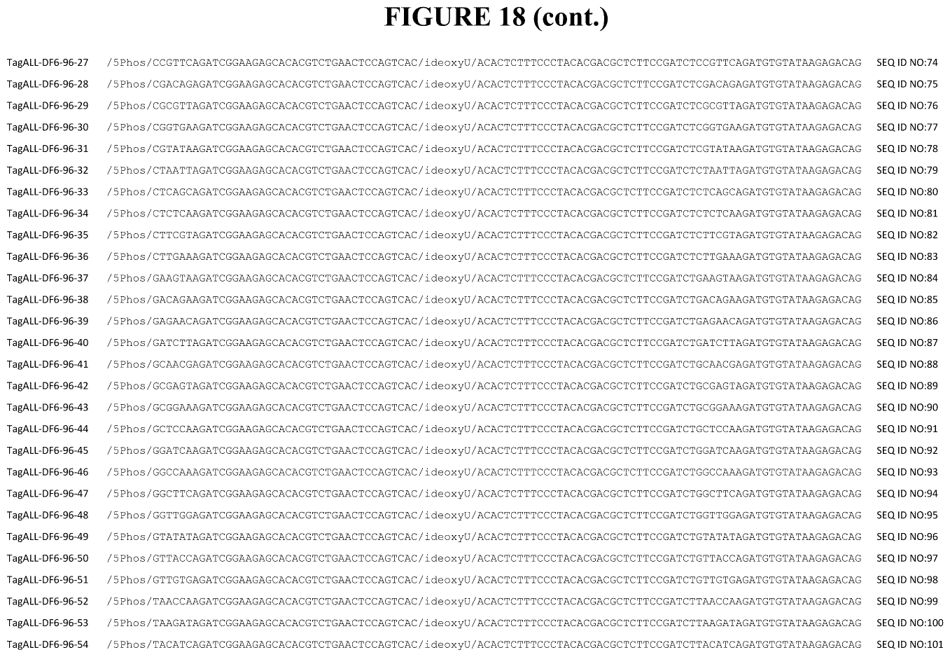

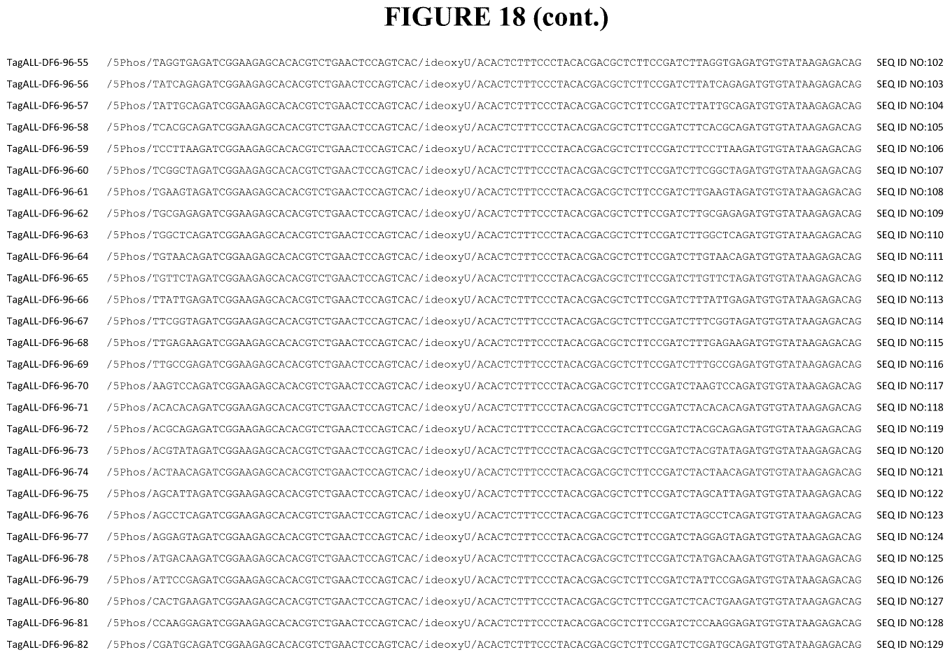

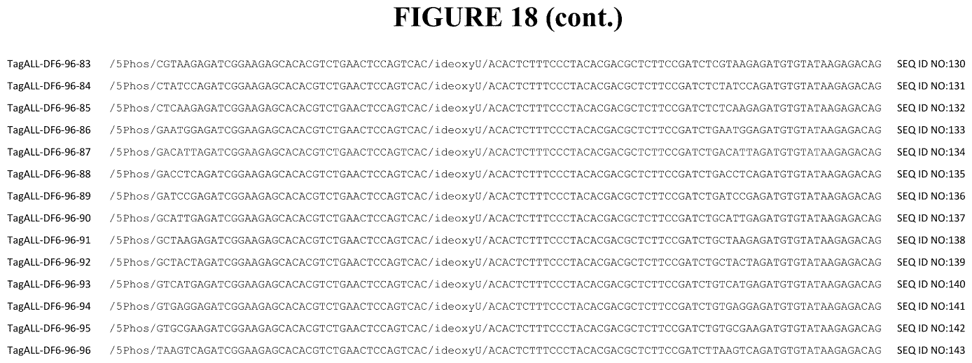

To demonstrate the feasibility of identifying paired duplex molecules, TagALL duplex libraries were generated using a defined pool of duplex transposons comprising 96 unique UMI pairs. Each duplex transposon contained a predefined association between a first UMI and a second UMI (see, UMI pairing table and corresponding oligonucleotide sequences in FIG. 18).

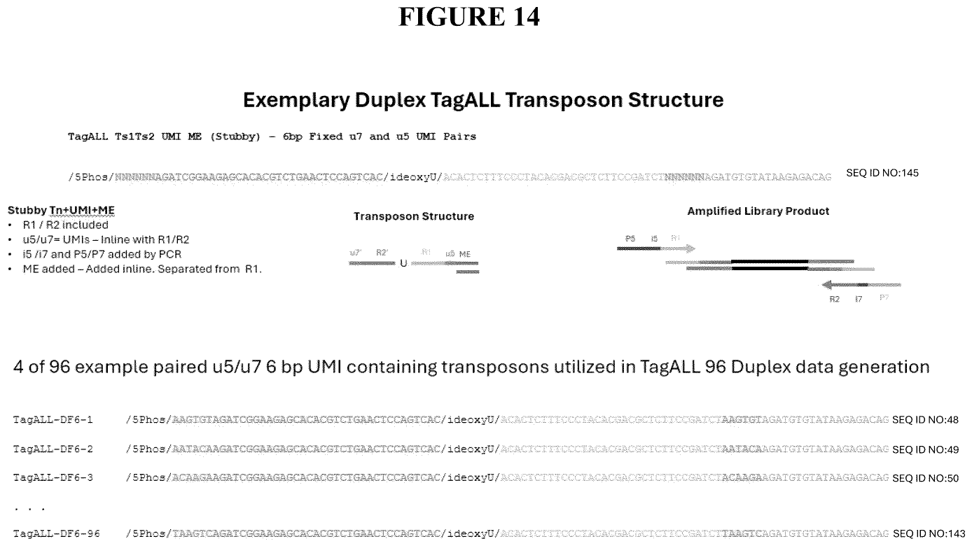

FIG. 14 illustrates the structure of an exemplary TagALL duplex transposon, including the placement of inline duplex UMIs within the adapter sequences and the associated transposon mosaic end (ME) sequence. In this embodiment, the paired UMIs are the same fixed sequence of 6 nucleotides, in a different orientation. A total of 96 distinct duplex transposons were utilized to generate the data described herein (see FIG. 18).

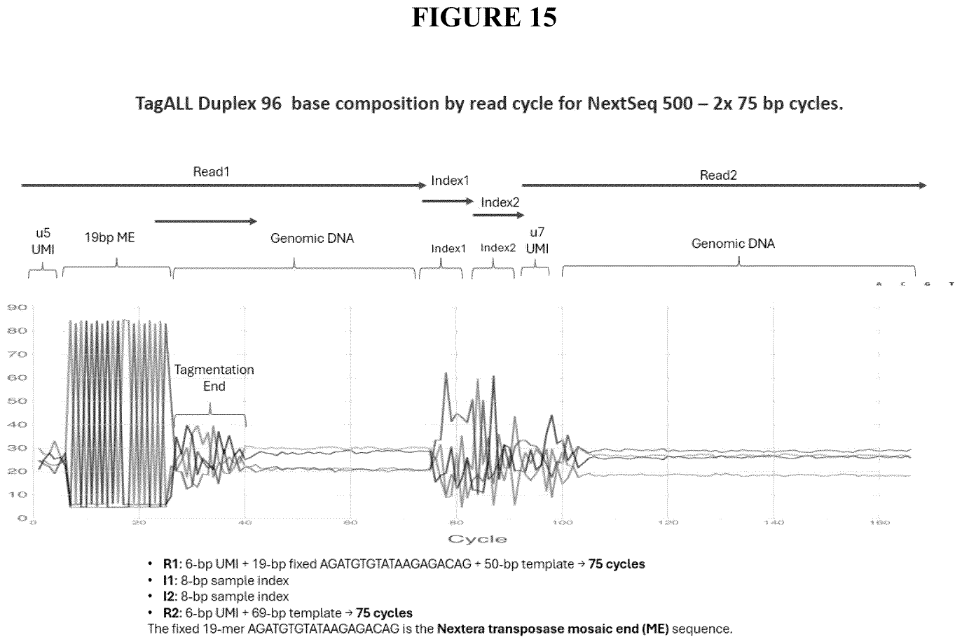

FIG. 15 shows the base composition of sequencing reads generated from a human genomic DNA library prepared using the TagALL duplex 96-UMI transposon pool and sequenced using a 2×75 bp Illumina sequencing run. The observed base composition across read cycles is consistent with successful library construction and confirms the expected read structure. Specifically, Read 1 exhibits base composition consistent with: i) an initial 6 bp UMI sequence (u5), ii) followed by a 19 bp transposon mosaic end (ME) sequence, and iii) followed by genomic insert sequence, wherein the first approximately 10-15 bases of the genomic insert display sequence bias consistent with transposase insertion preferences. Subsequent cycles correspond to sequencing of sample index sequences (e.g., index 1 and index 2). Read 2 begins at a later cycle (e.g., approximately cycle 91) and comprises: i) a second 6 bp UMI sequence (u7), and ii) followed by the opposite end of the genomic insert sequence.

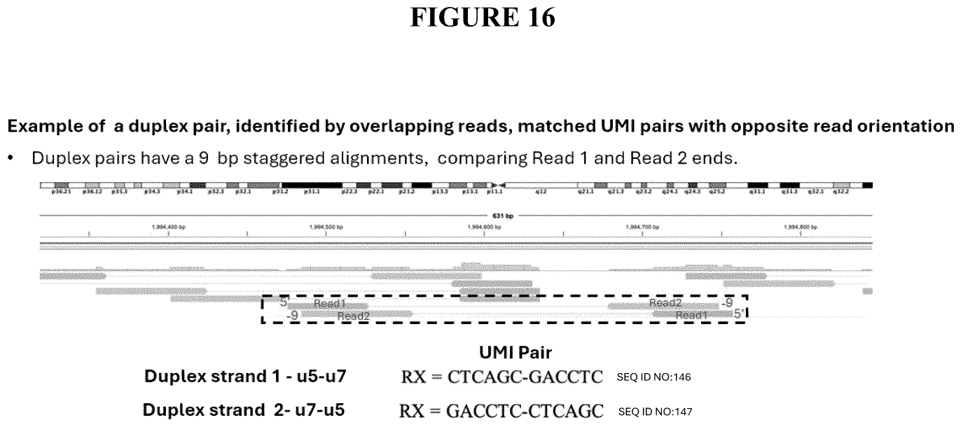

Duplex molecules were identified informatically by detecting paired-end reads corresponding to complementary strands of the same original duplex molecule. Such duplex-matched read pairs were identified based on: i) alignment to overlapping genomic coordinates, ii) opposite strand orientation of Read 1 and Read 2, and iii) and presence of corresponding UMI pairs (e.g., u5 and u7) consistent with duplex origin. For duplex-matched pairs, the relative alignment offset between the paired reads was assessed. FIG. 16 shows an example of a duplex molecule consisting of two paired-end reads derived from opposite strands, exhibiting: i) read alignments in opposite orientations between strands; ii) matching but oppositely orientated UMIs, and iii) an offset between aligned read positions consistent with transposase-mediated fragmentation. In this example, the observed offset is approximately 9 base pairs, consistent with the expected staggered cleavage pattern generated by Tn5 transposase.

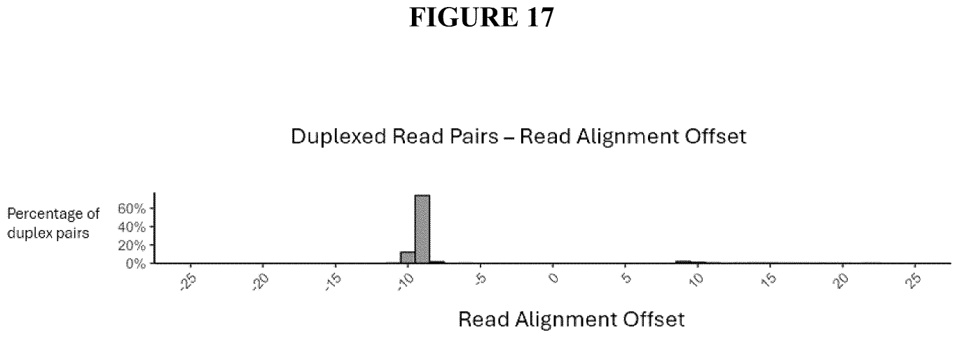

FIG. 17 shows the distribution of alignment offsets for all detected duplex molecules within a representative library. Greater than 60% of identified duplex molecules exhibit an offset of approximately 9 base pairs, consistent with the expected transposase-generated stagger and supporting accurate duplex pairing.

Together, these results demonstrate that TagALL duplex transposon designs with predefined UMI pairing enable reliable identification and reconstruction of duplex nucleic acid molecules.

### Methods

### Standard Tagmentation Library Preparation:

Transposome Assembly. Standard NGS transposon oligos (STD-Tn-oligoA+ME-rev) and (STD-Tn-oligoB+ME-rev) were annealed together at room temperature at a concentration of M each, for 30 minutes, in 1× Annealing Buffer (10 mM TRIS-HCL pH8.0, 50 mM NaCl and 0.1 mM EDTA). Oligo A and oligo B Transposons were then combine together at equal molar ratios. Active Transposomes were assembled by combining in equal molar ratio Tn5 Transposase (AlphaZyme, Jupiter FL) and annealed Transposon oligos, and diluted in Tris HCL pH8.0 Dilution Buffer and Glycerol to a final concentration of 3 M Transposome in 0.1 mM Tris HCL pH8.0, and 40% Glycerol. Combined Transposome was incubated at room temperature for 30 minutes, before storing at minus 20° C.
Library Construction. Standard Transposon-based Illumina compatible sequencing libraries were generated as follows: 200 ng of Human Genomics DNA was tagmented with between 3 and 12 pmol of standard (OligoA and OligoB) assembled Transposome, at 55° C. for 10 minutes, in a 1×DMF Buffer (10 mM Tris HCL pH8.0, 5 mM MgCl₂, 10% Dimethylformamide). The resulting Tagmentation was inactivated by treating with 0.4 μl of Heat liable proteinase (120 units/ml, NEB P8111S), incubating at 37° C. for 15 minutes, and 55° C. for 10 minutes, followed by a AMPure XP Bead clean up at a 2× bead to reaction ratio, washing with 80% ethanol and eluting in 20 μl of Elution Buffer (10 mM Tris-Cl, pH 8.0). 7.5 μl of tagmented material (approximately 75 ng of input) was use in a PCR amplification reaction, with KAPA HiFI master mix (Roche, K1121) and 5 μM of paired P5 and P7 indexing primers containing NxR1 and NxR2 sequences respectively (see PCR primers listed in Table 9). The PCR program run for Standard Transposon Libraries utilized initial extension and denaturation steps of 72° C. for 3 mins, and 95° C. for 30 seconds, followed by 12 cycles of 98° C. for 10 seconds, 55° C. for 30 seconds, 72° C. for 30 seconds, and a final extension step of 72° C. for 5 minutes. The PCR product was purified with a 0.8×AMPure XP clean up, and eluted in 20 ul of Elution Buffer.

### Tagmentation and Proximal Looped Ligation, Library Preparation.

Transposome Assembly. TagALL NGS transposon oligos, TPL1, TPL2, and TPL3 (Table 8) were independently annealed with ME-rev at room temperature at a concentration of 25 μM each, for 30 minutes, in 1× Annealing Buffer (10 mM TRIS-HCL pH8.0, 50 mM NaCl and 0.1 mM EDTA). Active Transposome were assembled by combining in equal molar ratio Tn5 Transposase (AlphaZyme, Jupiter FL) and annealed TSP1:ME-rev Transposon oligos, and diluted in Tris HCL pH8.0 Dilution Buffer and Glycerol to a final concentration of 3 M Transposome in 0.1 mM Tris HCL pH8.0, and 40% Glycerol. Combined Transposome was incubated at room temperature for 30 minutes, before storing at minus 20° C.

Library Construction. TagALL NGS Transposon-based Illumina compatible sequencing libraries were generated as follows: 200 ng of Human genomic DNA was tagmented with between 3 and 12 pmol of an assembled TagALL Transposome, at 55° C. for 10 minutes, in a 1×DMF Buffer (10 mM Tris HCL pH8.0, 5 mM MgCl₂, 10% Dimethylformamide). The resulting Tagmentation was inactivated by treating with 0.4 μl of Heat liable proteinase (120 units/ml, NEB P8111S), incubating at 37° C. for 15 minutes, and 55° C. for 10 minutes, followed by a AMPure XP Bead clean-up at a 2× bead to reaction ratio, washing with 80% ethanol and eluting int 20 μl of Elution Buffer (10 mM Tris-Cl, pH 8.0). 7.5 μl of tagmented material (approximately 75 ng of input) was utilized in downstream library steps to create individual libraries. 7.5 ul of material was heated to 95° C. for 5 minutes, then placed on ice, to create single stranded material and an alternative 7.5 ul of material was left as double stranded tagmented material. The double and single stranded aliquots were processed independently in subsequent steps. Next, tagmented material was circularized at 60° C. for 30 minutes with 0.5 μl Circligase enzyme (E096 Alpha-zyme) in 1× ligation Buffer, supplemented with 2.5 mM MnCl₂. The reactions were inactivated by treating with 0.2 μl of heat liable Proteinase K (120 units/ml, NEB P8111S), incubating at 37° C. for 15 minutes, and 55° C. for 10 minutes, followed by a AMPure XP bead clean up at a 2× bead to reaction ratio, washing with 80% ethanol and eluting int 24 μl of Elution Buffer (10 mM Tris-Cl, pH 8.0).

The eluted circularized material was then linearized with 2 ul of Uracil DNA Glycosylase (Alpha-zyme, E038-B002A) and 1 ul EndoVIII (Alpha-zyme E078-B059A), in 1× rCutsmart Buffer (NEB B6004S.), incubated at 37° C. for 15 minutes. The reactions was then purified using 2× ratio of AMPure XP beads to reaction, washing with 80% ethanol and eluting into 20 μl of Elution Buffer. The purified material was then PCR amplified with KAPA HiFI master mix (Roche, K1121) and 5 M of paired P5 and P7 indexing primers containing TSP Transposome compatible Illumina Read 1 and Read 2 sequences (see PCR primers listed in Table 9). The PCR program run for TagALL transposome utilized an initial denaturation steps of 95° C. for 30 secs, followed by 12 cycles of 98° C. for 10 seconds, 55° C. for 30 seconds, 72° C. for 30 seconds, and a final extension step of 72° C. for 5 minutes. The PCR product was purified with a 0.8×AMPure XP clean up, and eluted in 20 ul of Elution Buffer.

### Sequencing

All generated libraries were quantified on an Agilent Tapestation High Sensitivity Tape and by qPCR prior to pooling and sequencing on an Illumina NextSeq 500 (2×150 bp paired end, with 2×8 bp indexing reads) using standard Illumina reagents.

### Informatics

Sequencing data was demultiplexed, down-sampled and aligned in Basespace using FASTQ Generation v1.0.0, DRAGEN FASTQ Toolkit v1.3.1 and DRAGEN FASTQ Toolkit v1.3.1 aligning to hg38 human genome.

### Oligo Sequences:

TABLE 8

| Oligo Name | Sequence |
|---|---|
| TPL1 (Tn-Nx1Nx2) | /5Phos/CTGTCTCTTATACACATCTCCGAGC |
| (SEQ ID NO: 1) | CCACGAGAC/ideoxyU/TCGTCGGCAGCGTC |
| | AGATGTGTATAAGAGACAG |
| TPL2 (Nx1Ts2) | /5Phos/AGATCGGAAGAGCACACGTCTGAAC |
| (SEQ ID NO: 3) | TCCAGTCAC/ideoxyU/TCGTCGGCAGCGTC |
| | AGATGTGTATAAGAGACAG |
| TPL3 (Tn-Ts1Nx2) | /5Phos/AGATCGGAAGAGCGTCGTGTAGGGA |
| (SEQ ID NO: 4) | AAGAGTGT/ideoxyU/GTCTCGTGGGCTCGG |
| | AGATGTGTATAAGAGACAG |
| STD-Tn-oligoA | TCGTCGGCAGCGTCAGATGTGTATAAGAGACA |
| (SEQ ID NO: 5) | G |
| STD-Tn-oligoB | GTCTCGTGGGCTCGGAGATGTGTATAAGAGAC |
| (SEQ ID NO: 6) | AG |
| ME-rev | CTGTCTCTTATACACATCT |
| (SEQ ID NO: 2) | |

TABLE 9
PCR Amplification Primers

| Name | Sequence |
|---|---|
| P5-15-TSR1_DI50001 | AATGATACGGCGACCACCGAGATCTACACAGCGCTAGACACTCTTTCCCTACACGAC (SEQ ID NO: 7) |
| P5-15-TSR1_DI50002 | AATGATACGGCGACCACCGAGATCTACACGATATCGAACACTCTTTCCCTACACGAC (SEQ ID NO: 8) |
| P5-15-TSR1_DI50003 | AATGATACGGCGACCACCGAGATCTACACCGCAGACGACACTCTTTCCCTACACGAC (SEQ ID NO: 9) |
| P5-15-TSR1_DI50004 | AATGATACGGCGACCACCGAGATCTACACTATGAGTAACACTCTTTCCCTACACGAC (SEQ ID NO: 10) |
| P7-17-TSR2_DI70001 | CAAGCAGAAGACGGCATACGAGATAACCGCGGGTGACTGGAGTTCAGACGTGT (SEQ ID NO: 11) |
| P7-17-TSR2_DI70002 | CAAGCAGAAGACGGCATACGAGATGGTTATAAGTGACTGGAGTTCAGACGTGT (SEQ ID NO: 12) |
| P7-17-TSR2_DI70003 | CAAGCAGAAGACGGCATACGAGATCCAAGTCCGTGACTGGAGTTCAGACGTGT (SEQ ID NO: 13) |
| P7-17-TSR2_DI70004 | CAAGCAGAAGACGGCATACGAGATTTGGACTTGTGACTGGAGTTCAGACGTGT (SEQ ID NO: 14) |
| P5-15-NXR1_DI50005 | AATGATACGGCGACCACCGAGATCTACACAGGTGCGTTCGTCGGCAGCGTC (SEQ ID NO: 15) |
| P5-15-NXR1_DI50006 | AATGATACGGCGACCACCGAGATCTACACGAACATACTCGTCGGCAGCGTC (SEQ ID NO: 16) |
| P5-15-NXR1_DI50007 | AATGATACGGCGACCACCGAGATCTACACACATAGCGTCGTCGGCAGCGTC (SEQ ID NO: 17) |
| P5-15-NXR1_DI50008 | AATGATACGGCGACCACCGAGATCTACACGTGCGATATCGTCGGCAGCGTC (SEQ ID NO: 18) |
| P7-17-NXR2_DI70005 | CAAGCAGAAGACGGCATACGAGATCAGTGGATGTCTCGTGGGCTCGG (SEQ ID NO: 19) |
| P7-17-NXR3_DI70006 | CAAGCAGAAGACGGCATACGAGATTGACAAGCGTCTCGTGGGCTCGG (SEQ ID NO: 20) |
| P7-17-NXR4_DI70007 | CAAGCAGAAGACGGCATACGAGATCTAGCTTGGTCTCGTGGGCTCGG (SEQ ID NO: 21) |
| P7-17-NXR5_DI70008 | CAAGCAGAAGACGGCATACGAGATTCGATCCAGTCTCGTGGGCTCGG (SEQ ID NO: 144) |

Table 10 below lists Read1 and Read 2 sequencing primers that are compatible with Transposons TPL1, TPL2 and TPL3 as indicated. It is noted that Illumina supplies the sequencing primers as a mix and does not specifically disclose the sequence of these primers. As such, it is only presumed this is the sequences they provide.

TABLE 10
Presumed Sequencing Primers

| | | Transposon |
|---|---|---|
| Oligo Name | Sequence | Compatibility |
| Nx Read1 SP | TCGTCGGCAGCGTCAGATGTGT | TPL1 & TPL2 |
| | ATAAGAGACAG | |
| | (SEQ ID NO: 44) | |
| Nx Read2 SP | GTCTCGTGGGCTCGGAGATGTG | TPL1 & TPL3 |
| | TATAAGAGACAG | |
| | (SEQ ID NO: 45) | |
| Ts Read1 SP | ACACTCTTTCCCTACACGACGC | TPL3 |
| | TCTTCCGATCT | |
| | (SEQ ID NO: 46) | |
| Ts Read2 SP | GTGACTGGAGTTCAGACGTGTG | TPL2 |
| | CTCTTCCGATCA | |
| | (SEQ ID NO: 47) | |

### REFERENCES

1. Li, et al., (2020) Tn5 Transposase Applied in Genomics Research. Int J Mol Sci, 21, 8329.
2. Illumina Mate Pair https://www.illumina.com/science/technology/next-generation-sequencing/mate-pair-sequencing.html#:~:text=De %20Novo %20Assembly %20with %20Mate,of %20the %20final % 20consensus %20sequence.
3. Chen, et al. (2016) ATAC-see reveals the accessible genome by transposase-mediated imaging and sequencing. Nat Methods, 13, 1013-1020.
4. U.S. Pat. No. 11,530,436.
5. Xiong, et al., (2022) Duplex-Repair enables highly accurate sequencing, despite DNA damage. Nucleic Acids Res, 50, e1-e1.
6. U.S. Pat. No. 5,965,443
7. U.S. Pat. No. 6,437,109
8. U.S. Pat. No. 10,907,205
9. Pat. Pub. 2024/0026348
10. Pat. Pub. 2023/0340568
11. Pat. Pub. 2007/0178478
12. U.S. Pat. No. 10,184,122
13. WO2019/217785
14. EP Pat. EP3430154B1

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A kit or system comprising:
a) a first transposon strand comprising:
i) a first adapter sequence comprising a 5′ end and a free 3′ end, and a first transposon sequence,
ii) a first cleavable region which comprises a modified non-canonical base, and
iii) a second adapter sequence comprising a free 5′ end and 3′ end,
wherein said first cleavable region is between said first and second adapter sequences;
b) a second transposon strand comprising a sequence all or partially complementary to said first transposon sequence; and
c) a transposase.

2. The kit or system of claim 1, further comprising: d) a single strand ligase.

3. The kit or system of claim 1, further comprising: d) one or more enzymes that cleave said cleavable region.

4. The kit or system of claim 3, wherein said one more enzymes are selected from: an endonuclease, endonuclease V, endonuclease VIII, an endonuclease and uracil DNA glycosylase, and thermostable oxoguanine glycosylase (OGG).

5. The kit or system of claim 1, further comprising: d) an aqueous buffer.

6. The kit or system of claim 1, wherein said first and second adapter sequences do not, or do not substantially, hybridize to each other.

7. The kit or system of claim 2, wherein said single strand ligase enzyme is selected from: Circligase I, Circligase II, Mth DNA ligase, RtcB ligase from E. coli, thermostable RtcB, LoopLigase, Tph ssDNA ligase SDL, Hyperligase, T4 RNA ligase 1, and T4 RNA ligase 2.

8. The kit or system of claim 2, wherein said single strand ligase has step 3 ligase activity, but not step 2 adenylyl transfer activity.

9. The kit or system of claim 8, wherein said single strand ligase is a thermostable lysine-mutant ssDNA/RNA ligase.

10. The kit or system of claim 9, wherein said thermostable lysine-mutant is a mutated version of a precursor thermostable ssDNA/RNA ligase, wherein said precursor thermostable ssDNA/RNA ligase has a Motif I EKx(D/N/H)G, and wherein said thermostable lysine-mutant replaces K in said Motif I with any other amino acid or is selected from alanine (A), serine (S), cysteine (C), valine (V), threonine (T), and Glycine (G).

11. The kit or system of claim 1, wherein said 5′ end of said second adapter sequence is un-phosphorylated, and/or wherein said 5′ end of said second adapter sequence comprises a blocking group.

12. The kit or system of claim 1, wherein said first and/or second adapter sequences further comprise at least one element selected from: a flow cell attachment sequence, a unique barcode sequence, a non-unique barcode sequence, a sample-identifying index sequence, and a universal PCR amplification primer binding sequence.

13. The kit or system of claim 1, where said first and second adapter sequences each comprise a barcode sequence.

14. A kit or system comprising:
a) a first transposon strand comprising:
i) a first adapter sequence comprising a 5′ end and a free 3′ end, and a first transposon sequence,
ii) a first cleavable region, and
iii) a second adapter sequence comprising a free 5′ end and 3′ end,
wherein said first cleavable region is between said first and second adapter sequences;
b) a second transposon strand comprising a sequence all or partially complementary to said first transposon sequence;
c) a transposase; and
d) a single-strand ligase.

15. The kit or system of claim 14, further comprising: e) one or more enzymes that cleave said cleavable region.

16. The kit or system of claim 15, wherein said one more enzymes are selected from: an endonuclease, endonuclease V, endonuclease VIII, an endonuclease and uracil DNA glycosylase, and thermostable oxoguanine glycosylase (OGG).

17. The kit or system of claim 14, wherein said cleavable region comprises: A) a modified non-canonical base, which is optionally deoxyuridine, B) a nucleic acid sequence backbone linkage, which is optionally a phosphorothioate or a phosphodiester bond, or C) an endonuclease recognition site, wherein said endonuclease recognition site is formed at the junction of said first adapter 5′ end and said second adaptor 3′ end, or is composed of a sequence between said first and second adapter, or D) one or more RNA bases, wherein optionally the rest of the first and/or second transposon strands are composed of DNA, or E) comprises a sequence that forms a endonuclease recognition sequence when a secondary oligonucleotide is added.

18. The kit or system of claim 14, wherein said single strand ligase is selected from: Circligase I, Circligase II, Mth DNA ligase, RtcB ligase from E. coli, thermostable RtcB, LoopLigase, Tph ssDNA ligase SDL, Hyperligase, T4 RNA ligase 1, and T4 RNA ligase 2.

19. The kit or system of claim 14, wherein said single strand ligase has step 3 ligase activity, but not step 2 adenylyl transfer activity.

20. The kit or system of claim 14, wherein said single strand ligase is a thermostable lysine-mutant ssDNA/RNA ligase, and optionally wherein said thermostable lysine-mutant is a mutated version of a precursor thermostable ssDNA/RNA ligase, wherein said precursor thermostable ssDNA/RNA ligase has a Motif I EKx(D/N/H)G, and wherein said thermostable lysine-mutant replaces K in said Motif I with any other amino acid or is selected from alanine (A), serine (S), cysteine (C), valine (V), threonine (T), and Glycine (G).


SEQUENCE LISTING (147 sequences)

SEQ ID NO: 1 - DNA, 68 bp, Synthetic construct
  source 1..68: mol_type = other DNA
  misc_feature 1: note = 5' phosphorylation
  modified_base 35: mod_base = OTHER; note = deoxyuridine
ctgtctcttatacacatctccgagcccacgagacttcgtcggcagcgtcagatgtgtataagagacag

SEQ ID NO: 2 - DNA, 19 bp, Synthetic construct
  source 1..19: mol_type = other DNA
ctgtctcttatacacatct

SEQ ID NO: 3 - DNA, 68 bp, Synthetic construct
  source 1..68: mol_type = other DNA
  misc_feature 1: note = 5' phosphorylation
  modified_base 35: mod_base = OTHER; note = deoxyuridine
agatcggaagagcacacgtctgaactccagtcacttcgtcggcagcgtcagatgtgtataagagacag

SEQ ID NO: 4 - DNA, 68 bp, Synthetic construct
  source 1..68: mol_type = other DNA
  misc_feature 1: note = 5' phosphorylation
  modified_base 34: mod_base = OTHER; note = deoxyuridine
agatcggaagagcgtcgtgtagggaaagagtgttgtctcgtgggctcggagatgtgtataagagacag

SEQ ID NO: 5 - DNA, 33 bp, Synthetic construct
  source 1..33: mol_type = other DNA
tcgtcggcagcgtcagatgtgtataagagacag

SEQ ID NO: 6 - DNA, 34 bp, Synthetic construct
  source 1..34: mol_type = other DNA
gtctcgtgggctcggagatgtgtataagagacag

SEQ ID NO: 7 - DNA, 57 bp, Synthetic construct
  source 1..57: mol_type = other DNA
aatgatacggcgaccaccgagatctacacagcgctagacactctttccctacacgac

SEQ ID NO: 8 - DNA, 57 bp, Synthetic construct
  source 1..57: mol_type = other DNA
aatgatacggcgaccaccgagatctacacgatatcgaacactctttccctacacgac

SEQ ID NO: 9 - DNA, 57 bp, Synthetic construct
  source 1..57: mol_type = other DNA
aatgatacggcgaccaccgagatctacaccgcagacgacactctttccctacacgac

SEQ ID NO: 10 - DNA, 57 bp, Synthetic construct
  source 1..57: mol_type = other DNA
aatgatacggcgaccaccgagatctacactatgagtaacactctttccctacacgac

SEQ ID NO: 11 - DNA, 53 bp, Synthetic construct
  source 1..53: mol_type = other DNA
caagcagaagacggcatacgagataaccgcgggtgactggagttcagacgtgt

SEQ ID NO: 12 - DNA, 53 bp, Synthetic construct
  source 1..53: mol_type = other DNA
caagcagaagacggcatacgagatggttataagtgactggagttcagacgtgt

SEQ ID NO: 13 - DNA, 53 bp, Synthetic construct
  source 1..53: mol_type = other DNA
caagcagaagacggcatacgagatccaagtccgtgactggagttcagacgtgt

SEQ ID NO: 14 - DNA, 53 bp, Synthetic construct
  source 1..53: mol_type = other DNA
caagcagaagacggcatacgagatttggacttgtgactggagttcagacgtgt

SEQ ID NO: 15 - DNA, 51 bp, Synthetic construct
  source 1..51: mol_type = other DNA
aatgatacggcgaccaccgagatctacacaggtgcgttcgtcggcagcgtc

SEQ ID NO: 16 - DNA, 51 bp, Synthetic construct
  source 1..51: mol_type = other DNA
aatgatacggcgaccaccgagatctacacgaacatactcgtcggcagcgtc

SEQ ID NO: 17 - DNA, 51 bp, Synthetic construct
  source 1..51: mol_type = other DNA
aatgatacggcgaccaccgagatctacacacatagcgtcgtcggcagcgtc

SEQ ID NO: 18 - DNA, 51 bp, Synthetic construct
  source 1..51: mol_type = other DNA
aatgatacggcgaccaccgagatctacacgtgcgatatcgtcggcagcgtc

SEQ ID NO: 19 - DNA, 47 bp, Synthetic construct
  source 1..47: mol_type = other DNA
caagcagaagacggcatacgagatcagtggatgtctcgtgggctcgg

SEQ ID NO: 20 - DNA, 47 bp, Synthetic construct
  source 1..47: mol_type = other DNA
caagcagaagacggcatacgagattgacaagcgtctcgtgggctcgg

SEQ ID NO: 21 - DNA, 47 bp, Synthetic construct
  source 1..47: mol_type = other DNA
caagcagaagacggcatacgagatctagcttggtctcgtgggctcgg

SEQ ID NO: 22 - DNA, 19 bp, Synthetic construct
  source 1..19: mol_type = other DNA
agatgtgtataagagacag

SEQ ID NO: 23 - protein, 380 aa, Synthetic construct
  VARIANT 92: note = X is any amino acid except K
MVSSYFRNLLLKLGLPEERLEVLEGKGALAEDEFEGIRYVRFRDSARNFRRGTVVFETGEAVLGFPHIKRVVQLENGIRRVFKNKPFYVEEXVDGYNVRVVKVKDKILAITRGGFVCPFTTERIEDFVNFDFFKDYPNLVLVGEMAGPESPYLVEGPPYVKEDIEFFLFDIQEKGTGRSLPAEERYRLAEEYGIPQVERFGLYDSSKVGELKELIEWLSEEKREGIVMKSPDMRRIAKYVTPYANINDIKIGSHIFFDLPHGYFMGRIKRLAFYLAENHVRGEEFENYAKALGTALLRPFVESIHEVANGGEVDETFTVRVKNITTAHKMVTHFERLGVKIHIEDIEDLGNGYWRITFKRVYPDATREIRELWNGLAFVD

SEQ ID NO: 24 - protein, 379 aa, Synthetic construct
  VARIANT 92: note = X is any amino acid except K
MVSSHFKEILMRLGLPEDRIEVLEAKGGITEEEFDGIRYLRFKDSARGLRRGTVVFDEANVILGFPHIKRVVSLRAGVMRIFKRTPFYVEEXVDGYNVRVALVSDRVLAITRGGFVCPFTTERILDFVPEEFFKDYPHLVLVGEMAGPESPYLVEGPPYVEEDIRFFLFDIQEKGTGKSLPVQERLKLAEEYGIPHVKVFGLYTVDRIEDLYDLIERLSREGREGVVMKSPDMKRVVKYVTPFANVNDVKIGAKVFFELPPGYFMSRIMRLAFYVAERRIKGERFEELARNLGKALLEPFVESIWDVEQGDEIAEVFRIRVKRIETAYKMVTHFERLGLNIKIEDIEEVGGMWRITFKRAYDEATREIRELIGGRAFVD

SEQ ID NO: 25 - protein, 379 aa, Synthetic construct
  VARIANT 92: note = X is any amino acid except K
MVSSKFKDILYRLGIPEGKVEDLEARGGLVEDKFDDIKYLRIRNSVGKLRRGTVVLNDKFIILGFPHIKRIVNLKNGIKRTFKRGEFYVEEXVDGYNVRVVKFRGKILGITRGGFICPFTTERISDFIPEEFFKDHPNLILVGEMAGPESPYLVEGPPYVKEDIQFFLFDIQELGTGRSLPVEERLKIAEEYGISHVEVFGKFTYKDLEEIYEIVERLSREGREGIVMKSPDMRKMVKYVTPYANINDIKIGARVFYELPPGYFTSRISRLAFYIAEKRLRGENFEELAKELGKALLQPLVESIHDVEQEDEIAEVFKVRVKKIETAYKMVTHFEKLGLRIEIVDIEEMKGGWRITFKRLYPDATEEIRELIGGKSFVD

SEQ ID NO: 26 - protein, 382 aa, Synthetic construct
  VARIANT 95: note = X is any amino acid except K
MKEVVSSVYKEILVKLGLTEDRIETLEMKGGIIEDEFDGIRYVRFKDSAGKLRRGTVVIDEEYVIPGFPHIKRIINLRSGIRRIFKRGEFYVEEXVDGYNVRVVMYKGKMLGITRGGFICPFTTERIPDFVPQEFFKDNPNLILVGEMAGPESPYLVEGPPYVKEDIQFFLFDVQEIKTGRSLPVEERLKIAEEYGINHVEVFGKYTKDDVDELYQLIERLSKEGREGIIMKSPDMKKIVKYVTPYANINDIKIGARVFYELPPGYFTSRISRLAFYLAEKRIKGEEFERVAKELGSALLQPFVESIFDVEQEEDIHELFKVRVKRIETAYKMVTHFEKLGLKIEIVDIEEIKDGWRITFKRLYPDATNEIRELIGGKAFVD

SEQ ID NO: 27 - protein, 382 aa, Synthetic construct
  VARIANT 95: note = X is any amino acid except K
MENMVSSKFKELLYTLGIPEDKVEILEARGGIMEDEFEGIRYLRFKNSVGKLRRGTVLFEDGTTVFGFPHIKRIVNLSAGVRKIFKSSEFYVEEXVDGYNVRVVKFKDRILGITRGGFICPYTTERIAEFVPEEFFKDHKDLVLVGEMAGPESPYLVEGPPYVKEDIQFFLFDIQDIKTGSSLPVEERLKLAEEYGINHVEVFGRYSYKDIDDLYELIERLSREGREGIVMKSPDMKKIVKYVTPYANINDIKIGARVFYELPGGYFTSRISRLAFYIAEKKIRGEELHNLALQLGKALLQPLVEAIHDVTQGDVIAERFRVRVRKIETAYKMVTHFEKLGLEIEIEDIEEIEGGWRVTFKRVYPEATREIRDLIGGKAFVD

SEQ ID NO: 28 - protein, 390 aa, Synthetic construct
  VARIANT 100: note = X is any amino acid except K
MTWIKNPEPWMVNLVAEKLGLDVERVETLARHGTIRFRGYRDVVYALLRREIAGHPEGTVVLLERNGARLVPGYPPIQRMVLPTIALPRHFIDKVVVEEXMNGYNVRLVMFHRKLLAVTRGGFICPYTTARLERLIGGRVRELFREIDPETYTIAGEVVGLENPYTRYFYPEAPRFDYFVFDLFHELKPLPPLERNELLEKYGLKHVRLLGVIDKNDVEMFKQIVAELDREGREGVVAKDPEYRVPPLKYTTSAVNIGDVRYGMRFFMEEGRSFLFSRLLRELFRAYEEGFGDAQLEKLALEFGRAATEPALESIRKVAMGDMLYEEFELVFADEVELEEFTSYMAELGVDIVVVSTSREDEGLRARMRKIKDTWIQLRKVLDTGLSPVD

SEQ ID NO: 29 - protein, 390 aa, Synthetic construct
MTWIKNPEPWMVNLVAEKLGLDVERVETLARHGTIRFRGYRDVVYALLRREIAGHPEGTVVLLERNGARLVPGYPPIQRMVLPTIALPRHFIDKVVVEEAMNGYNVRLVMFHRKLLAVTRGGFICPYTTARLERLIGGRVRELFREIDPETYTIAGEVVGLENPYTRYFYPEAPRFDYFVFDLFHELKPLPPLERNELLEKYGLKHVRLLGVIDKNDVEMFKQIVAELDREGREGVVAKDPEYRVPPLKYTTSAVNIGDVRYGMRFFMEEGRSFLFSRLLRELFRAYEEGFGDAQLEKLALEFGRAATEPALESIRKVAMGDMLYEEFELVFADEVELEEFTSYMAELGVDIVVVSTSREDEGLRARMRKIKDTWIQLRKVLDTGLSPVD

SEQ ID NO: 30 - protein, 385 aa, Synthetic construct
  VARIANT 99: note = X is any amino acid except K
MASAAEVLASALRAVGVDPGSVDLEALSTRRSVRVSRFEDVVYVGFRRQFRGVPEGTLVAFRRGEQIVVWGYPSIKRMLLPRVAVPRWFPGPTVLVEEXMNGYNVRVFTLGGMVYAATRGGLICPYTTRRLRRLYGGALQKILEDLGAEGSFIAGEVVGLENPYTRYYYEEAPGFGYFIFDIFKGGRQLPPRVKFSLAPEYGLKTVNLLAEIPATASGVERLYTIVEDLEKRGREGVIVKDPEGRVEPLKYTTSRTNIGDIRLGMRYPFEEGRSFLFPRILREIFREWETGRRRYGELGEAILAPAIEAVEAVSRGGRLVEEFELVFANEVEAEEVIAYFASLGVHLEIAGVARGVDGVRVAFRKPRKSEGEIARILETGISPLD

SEQ ID NO: 31 - protein, 381 aa, Synthetic construct
  VARIANT 92: note = X is any amino acid except K
MDENELVNKLSDALGIEYEKLSKHIGRSIRLMKYGELNYVVFRRDLLGYREGTTILLGEEPLIVHGYPSIQRLAFIEGVSKHMIDNVVVEEXMNGYNVRVVYYMNNIYAITRGGYICPYTTARIRKLYSKNIKLAYQEYPDTILVGEVVGTENPYVIYDYPEARGFDYFIFDTMKKDKLQPLRIRDEIAEKYSLKTVRILDIINKRDIDRLKTIINRLEKERREGVVLKDPYQRVPPLKYTTIYINIRDIWEGMRYPFDEGRGYLFSRIVRLIAQGYEYDWNNTELDRIALKLGRAILEPAINSLKKRANGEIIASKYTLVFPSEDDLSKYIEYAESIGMDFIFRVVEKREDGCIVVELFKMKETHNIYTKMLKTGYSPLD

SEQ ID NO: 32 - protein, 390 aa, Synthetic construct
  VARIANT 100: note = X is any amino acid except K
MIRIPLERWMIEKLAEALNVNIEEAERLARRRNVVRLMKWRNVTYFSLRKDVYGLREGTLIAVWPDGYRVVPGYPSIQRVLLPSVALPKHFIDKIVVEEXLNGYNVRVVKLRDEIVAVTRGGLICPYTTQRIRKLYGDKLTSLFREEGEELVVAGEVIGLENPYVRFYYPEAGGFAYFIFDIVHGEKFLPPHERKEIVEKHGLLHVPVLGEIDKNDIKAFRKIIEDLERRGREGVVLKDPEYRVPPLKYTTSFINIHDIEIGMRFPFDEGRNYLFSRILREIFKAVEEGWDDRRLLLAEQNLGKAILEPAIEAVKEVKNGKMLYEEFMLPFDTRDDFEEFLDYMASLGVDIIVAGVEQRSDGSIVARIRKVKDTWREVQKILETGLSPID

SEQ ID NO: 33 - protein, 367 aa, Synthetic construct
  VARIANT 77: note = X is any amino acid except K
MISPELVKEALKKKKVRSEEAFGLEYLRFNDDYKDIPRGTAIFKDFIIWGYPHIGRIFLLETGLREQFEAPFWVEEXVDGYNTRIFKYGDNYYALSRGGFICPFTTDRLPDLIDLRILDENPDLVICAEVAGPENPYIEESPPYVKEDVQLFVFDFMKKNEQGFLSQEEKMELIEKYNLPHVEILGRFTASEEGIKKIKEILKRFNEEGREGVVFKEDSERNKRAKYITSYANLMDIKTNAKNMLQLPPEYYTNRILRLVLFMYEEGLKTTEHLYEELGRAFIDGLFQAIEQFEKEHKVYKTFTCKFRKKENAIALLELLSKTSKHIQVKERRLEKEGDYWRLEFDKVFLNMTGLLGHLLSGGIVYD

SEQ ID NO: 34 - protein, 390 aa, Synthetic construct
  VARIANT 100: note = X is any amino acid except K
MTWIHSPESWMLDVVAEALGIDRERVEHLARHRTIRYRVFRGILYASLRREVAGHPEGTVIVFGRGWWRLIPGYPSIQRMVLPSVALPRHFVDKIVVEEXLNGYNVRVALIDDRIIAVTRGGFICPYTTSRLERIMGNQLKDMLRELGPEEHVAAGEVIGLENPYTRYFYPEAPRFGYFVFDVFREGKPLPPGWRDEVTEKHGVPHVPVLGVLDKNDIEGFKKIVERLNQEGREGVIVKDPEYRVPPLKYTTPATNIGDIRYGMRFFMEEGRGFLFSRLLREIFRVYEEGLTGPRLDALALELGRAALQPAIETVKKVAAGDMVYEEFELEFASRSELEEFMDYMQGLGVDLVLVEIREENGLLKTRIRKMKETWLQVRKMLETGLTPID

SEQ ID NO: 35 - protein, 388 aa, Synthetic construct
  VARIANT 101: note = X is any amino acid except K
MRRDVSQFANKLDIGKVSELLDIPEHRITGALKRKTIQYVWGKKELFRFDKPVSSIEGGTSVFTEPFDIVRGFPKISRTLMLSPALQKHFSSCRKVAVEEXMNGYNVRVALIGDALVALTRGGFICPYTTEKAIDLIGYDFFNDHPDLVLCGEMVGPDSPYVPKTFYDIESLDFFVFDIREKITGKPLSVMERRALVDKYGIKSVRLFGEFEIGETHSEITRIIKDLGGSQHEGVVIKDPQMVVPPMKYTSSESNCADLRYAFEFYNDFGRDFFFGRVCREAFQSVEWDEDEESVEKRCRQLGESLLLPMIKTIKKKKDGERIAENVQIRVKSLDTVKEFEEYLKLVGVDAVFEEPEQTGNEYFVRIRKMHQSTNDRTEAILGGQLWS

SEQ ID NO: 36 - protein, 390 aa, Synthetic construct
  VARIANT 100: note = X is any amino acid except K
MTWIHRPEPWMLDVVADALGLPRERVEELASRRTLRFREFRGLLYASLRRGVAGHHEGTAVVFGRGWWRVVPGYPPIQRMVLPSVALPRHFLDRVVVEEXLNGYNVRVVLVDDRILAVTRGGLICPYTTSRLERLMGDRLREMLRELGPEDHVAAGEVIGLENPYTRYFYPEAPRFGYFVFDIFRGGRPLPPRMRDEAAEKHGVPHVPVLGVLEKTDVEAFKRIVERLDREGREGVVVKDPDYRVPPLKYTTSSTNIGDIRLGMRFFMEEGWSFLFSRILREIFRVYEEGVEGPRLDAIALELGRAALQPAVETVKKVAGGYMVYEEFELEFAGRDELEEFMDYMQSLGVDVVLVEAREEGGVLRARMRKIKETWIRVRRILETGVSPID

SEQ ID NO: 37 - protein, 389 aa, Synthetic construct
  VARIANT 99: note = X is any amino acid except K
MGWVQPEPWMVDAVAEALGLERERVESLAKHRTIRFRVFRGILYASLRRELGGYPEGTVVIFGRGWSRVVHGYPPIQRMVLPSVALPRHFVDRIVVEEXLNGYNVRVVLVDGRLLAVTRGGFICPYTTDRIERLLGGRLREMLRELGEEEHVAAGEVIGLENPYTRYYYPEAPRFGYFVFDIFRSGKPLPPRVRDEATEKHGVPHVPVLGVLDKGDIEGFRSIVEALERRGREGVVVKDPEYRVHPLKYTTHATNVGDIRLGMRFFMEEGRGFLFSRLLREIFRAYEQGLQGPRLEKLATEIGLAALEPALETVRLVAAGEPVYEEFELEFENRDRLEEFLEYMQSLGVDVVVAGTYERDGMLVARVRKMRDTWLQVRRMLETGLTPID

SEQ ID NO: 38 - protein, 380 aa, Synthetic construct
  VARIANT 94: note = X is any amino acid except K
MFVSESLGLSKHLGETLEERKILREALISHSFFSDVIEAVRFDKKFGEIEEGTVVAKTINGVRIVRGFPKIKRALVLNPTLKKHFENEVAVEEXMNGYNVRIARFGKNLYAMTRRGIICPYTTEKARELINPEFFKDHSDLVLCCEAVGEESPYVPKSMYGVEGLDFFVFDIREERTNRPLPVEEKLRLCEEYGLRHATYFGTYDVDVAHDEIKDIISDLAGKGREGVVIKDPEMKLSPLKYTTSQTNAEDLKYAFRFFNDYGKDFMFSRIVREGFQSFEFNEGDKEFRERCLRLGMAILKPMVESIREVALGGKVSEKLRLRFGSLDVMNLFFEQWKRSKVDFEITDIKKDGKDIVVFVNKTMRNTTDKIKAHLEGIPW

SEQ ID NO: 39 - protein, 380 aa, Synthetic construct
  VARIANT 95: note = X is any amino acid except K
MKFIAEALGVSQAVIEKLNEKNLIRLAFIKHPFFRDVIEAYKLERKVGEFEPGTLIAKTVEGLRVVRGYPKIKRALTLYPTIKKHFKGEVVLEEXMNGYNVRLVKFGENIYAITRGGFICPYTTEKARRLVNLDFFKDNPKLMLCCEAVGEESPFVPKDVYGVKTIDFYVFDIRDQKTNIALPIKQKEKLAEEYGLKLAPILAEVQVSKAHEIAKEIILELDKRGREGIVIKDPMMRRPPIKYTTSQCNCSDLSYAFRFFEEYGKDFMFSRIIREAFQSFEFRENEEKFKDRCLRLGEAILSMVKSIKEVNEGKRIVEKMRLRFYDLEIFELFKEHIRRMGIRAEFSNPKREEDGYVVWVYRHIMSTTDKIKYILAGNLY

SEQ ID NO: 40 - protein, 380 aa, Synthetic construct
  VARIANT 92: note = X is any amino acid except K
MVSSHFKSLLLELGISRERIEILESKGGIVEDEFEGIRYLRFKDSAGSLRRGTVVFDSHNIILGFPHIKRVVHLENGIKRVFKRKPFYVEEXVDGYNIRVAQIEGRVFAFTRGGFVCPFTTERIEDFVNMEFFKDYPNLVLCGEMAGPESPYLVEGPPYVKEDIEFFLFDIQEKKTGKSLTVEERLKIAEEYGIPSVEVFGVYDISKIDELKELIEQLSREKREGIVMKSPDMKKIVKYVTPYANVNDIKIGARIFFDLPHGYFMQRIKRLAFYLAEKRVQDEEFEKYARALGRALLEPFVESIWDVSAGEEIAEVFTVRVKHIETAYKMVSHFERLGLKIHIEDIEEMPQGYWRITFKRVYPDATREIRELWSGHAFVD

SEQ ID NO: 41 - protein, 379 aa, Synthetic construct
  VARIANT 92: note = X is any amino acid except K
MVSSRFKDILTSLGISEERIEILEAKGGIVEDEYEGLRYLRFKDSAGKLRRGTVVFDFDKIILGFPHIKRVVNLEKGIRRIFKRGEFYVEEXVDGYNVRVTKVGERILAITRGGFICPFTTERITDFVPEEFFKDNPNLVLVGEMAGPESPYLVEGPPYVKEDIKFFLFDVQEINTGKSLPVEERLKLAEEYGIPHVEVFGKYTRDDIGELYALIEKLSEEGREGIVMKSPDMKKIVKYVTPYANINDIKIGARVFYELPPGYFTSRISRLAFYIAERKIRDEELRKLAEDLGKALLQPFVEGILDVEQGEEIAETFKIRVKKIETAYKMVTHFEKLGLNIEIVDIEEMDGLWRITFKRVYSDATEKIKELVGGKAFVD

SEQ ID NO: 42 - protein, 361 aa, Synthetic construct
  VARIANT 67: note = X is any amino acid except K
MKSERGIMKYKDFIYYPFKKGGFGKGSVIIYHNDDVKIVPGYPSIKRLVLLSKVPEHFPEGVSVEEXMNGYNVRAMIVGGDVAFITRGGYLCPYTNARLNTLYGEKVKALLEELPPGSFLAGEVVGVENPYVRVKYPEAPYFDYFIFDIFVKTEDGWRQMPVEERHEIVKRHGLRSVRLLGTFESSEAPLKIKEIIDRFDKEGREGVVMKDPEYKRSPAKYTGSYTNIGDIREGMRYPFDEGKDYLFPRIVREIFKVYEEGLSDKELERRALELGMAILKPAVESLKEVAQGETLFERFVLRFPHEEDLEEYLNYTRSLGVKVIVEEKWEEGEWIVVKAKKFKNTSNVYRSMLKSGQTPLD

SEQ ID NO: 43 - protein, 381 aa, Synthetic construct
  VARIANT 92: note = X is any amino acid except K
MVSSYFKGILLNLGLDEERIEVLENKGGIVEDEFEGMRYLRLKDSARSLRRGTVVFDEHNIILGFPHIKRVVQLENGIRRAFKRKPFYVEEXVDGYNVRVAKIGEKILVFTRGGFVCPFTTERIEDFITLDFFKDYPNMVLCGEMAGPESPYLVEGPPYVKEDIQFFLFDIQEKKTGRSLPVEERLKLAEEYGIPSVEVFGLYDLSRIDELHALIDRLTKEKREGIVMKSPDMKKIVKYVTPYANINDIKIGARIFFDLPHGYFMQRIKRLAFYLAERKIRGEEFDEYARALGKVLLEPFVESIWDISSGDDEIAELFTVRVKKLETAHKMVTHFERLRLKIHIDDIEVLDNGYWRITFKRVYPDATKEMRELWNGHAFVD

SEQ ID NO: 44 - DNA, 33 bp, Synthetic construct; identical to SEQ ID NO: 5
  source 1..33: mol_type = other DNA

SEQ ID NO: 45 - DNA, 34 bp, Synthetic construct; identical to SEQ ID NO: 6
  source 1..34: mol_type = other DNA

SEQ ID NO: 46 - DNA, 33 bp, Synthetic construct
  source 1..33: mol_type = other DNA
acactctttccctacacgacgctcttccgatct

SEQ ID NO: 47 - DNA, 34 bp, Synthetic construct
  source 1..34: mol_type = other DNA
gtgactggagttcagacgtgtgctcttccgatca

SEQ ID NO: 48 - DNA, 99 bp, Synthetic construct
  source 1..99: mol_type = other DNA
  misc_feature 1: note = 5' phosphorylation
  modified_base 41: mod_base = OTHER; note = deoxyuridine
aagtgtagatcggaagagcacacgtctgaactccagtcactacactctttccctacacgacgctcttccgatctaagtgtagatgtgtataagagacag

SEQ ID NO: 49 - DNA, 99 bp, Synthetic construct
  source 1..99: mol_type = other DNA
  misc_feature 1: note = 5' phosphorylation
  modified_base 41: mod_base = OTHER; note = deoxyuridine
aatacaagatcggaagagcacacgtctgaactccagtcactacactctttccctacacgacgctcttccgatctaatacaagatgtgtataagagacag

SEQ ID NO: 50 - DNA, 99 bp, Synthetic construct
  source 1..99: mol_type = other DNA
  misc_feature 1: note = 5' phosphorylation
  modified_base 41: mod_base = OTHER; note = deoxyuridine
acaagaagatcggaagagcacacgtctgaactccagtcactacactctttccctacacgacgctcttccgatctacaagaagatgtgtataagagacag

SEQ ID NO: 51 - DNA, 99 bp, Synthetic construct
  source 1..99: mol_type = other DNA
  misc_feature 1: note = 5' phosphorylation
  modified_base 41: mod_base = OTHER; note = deoxyuridine
acatccagatcggaagagcacacgtctgaactccagtcactacactctttccctacacgacgctcttccgatctacatccagatgtgtataagagacag

SEQ ID NO: 52 - DNA, 99 bp, Synthetic construct
  source 1..99: mol_type = other DNA
  misc_feature 1: note = 5' phosphorylation
  modified_base 41: mod_base = OTHER; note = deoxyuridine
accgcaagatcggaagagcacacgtctgaactccagtcactacactctttccctacacgacgctcttccgatctaccgcaagatgtgtataagagacag

SEQ ID NO: 53 - DNA, 99 bp, Synthetic construct
  source 1..99: mol_type = other DNA
  misc_feature 1: note = 5' phosphorylation
  modified_base 41: mod_base = OTHER; note = deoxyuridine
acctggagatcggaagagcacacgtctgaactccagtcactacactctttccctacacgacgctcttccgatctacctggagatgtgtataagagacag

SEQ ID NO: 54 - DNA, 99 bp, Synthetic construct
  source 1..99: mol_type = other DNA
  misc_feature 1: note = 5' phosphorylation
  modified_base 41: mod_base = OTHER; note = deoxyuridine
actctgagatcggaagagcacacgtctgaactccagtcactacactctttccctacacgacgctcttccgatctactctgagatgtgtataagagacag

SEQ ID NO: 55 - DNA, 99 bp, Synthetic construct
  source 1..99: mol_type = other DNA
  misc_feature 1: note = 5' phosphorylation
  modified_base 41: mod_base = OTHER; note = deoxyuridine
actggtagatcggaagagcacacgtctgaactccagtcactacactctttccctacacgacgctcttccgatctactggtagatgtgtataagagacag

SEQ ID NO: 56 - DNA, 99 bp, Synthetic construct
  source 1..99: mol_type = other DNA
  misc_feature 1: note = 5' phosphorylation
  modified_base 41: mod_base = OTHER; note = deoxyuridine
agaggcagatcggaagagcacacgtctgaactccagtcactacactctttccctacacgacgctcttccgatctagaggcagatgtgtataagagacag

SEQ ID NO: 57 - DNA, 99 bp, Synthetic construct
  source 1..99: mol_type = other DNA
  misc_feature 1: note = 5' phosphorylation
  modified_base 41: mod_base = OTHER; note = deoxyuridine
agcaccagatcggaagagcacacgtctgaactccagtcactacactctttccctacacgacgctcttccgatctagcaccagatgtgtataagagacag

SEQ ID NO: 58 - DNA, 99 bp, Synthetic construct
  source 1..99: mol_type = other DNA
  misc_feature 1: note = 5' phosphorylation
  modified_base 41: mod_base = OTHER; note = deoxyuridine
aggatgagatcggaagagcacacgtctgaactccagtcactacactctttccctacacgacgctcttccgatctaggatgagatgtgtataagagacag

SEQ ID NO: 59 - DNA, 99 bp, Synthetic construct
  source 1..99: mol_type = other DNA
  misc_feature 1: note = 5' phosphorylation
  modified_base 41: mod_base = OTHER; note = deoxyuridine
aggcatagatcggaagagcacacgtctgaactccagtcactacactctttccctacacgacgctcttccgatctaggcatagatgtgtataagagacag

SEQ ID NO: 60 - DNA, 99 bp, Synthetic construct
  source 1..99: mol_type = other DNA
  misc_feature 1: note = 5' phosphorylation
  modified_base 41: mod_base = OTHER; note = deoxyuridine
agtgcgagatcggaagagcacacgtctgaactccagtcactacactctttccctacacgacgctcttccgatctagtgcgagatgtgtataagagacag

SEQ ID NO: 61 - DNA, 99 bp, Synthetic construct
  source 1..99: mol_type = other DNA
  misc_feature 1: note = 5' phosphorylation
  modified_base 41: mod_base = OTHER; note = deoxyuridine
agttaaagatcggaagagcacacgtctgaactccagtcactacactctttccctacacgacgctcttccgatctagttaaagatgtgtataagagacag

SEQ ID NO: 62 - DNA, 99 bp, Synthetic construct
  source 1..99: mol_type = other DNA
  misc_feature 1: note = 5' phosphorylation
  modified_base 41: mod_base = OTHER; note = deoxyuridine
atactcagatcggaagagcacacgtctgaactccagtcactacactctttccctacacgacgctcttccgatctatactcagatgtgtataagagacag

SEQ ID NO: 63 - DNA, 99 bp, Synthetic construct
  source 1..99: mol_type = other DNA
  misc_feature 1: note = 5' phosphorylation
  modified_base 41: mod_base = OTHER; note = deoxyuridine
atcataagatcggaagagcacacgtctgaactccagtcactacactctttccctacacgacgctcttccgatctatcataagatgtgtataagagacag

SEQ ID NO: 64 - DNA, 99 bp, Synthetic construct
  source 1..99: mol_type = other DNA
  misc_feature 1: note = 5' phosphorylation
  modified_base 41: mod_base = OTHER; note = deoxyuridine
atggacagatcggaagagcacacgtctgaactccagtcactacactctttccctacacgacgctcttccgatctatggacagatgtgtataagagacag

SEQ ID NO: 65 - DNA, 99 bp, Synthetic construct
  source 1..99: mol_type = other DNA
  misc_feature 1: note = 5' phosphorylation
  modified_base 41: mod_base = OTHER; note = deoxyuridine
attaagagatcggaagagcacacgtctgaactccagtcactacactctttccctacacgacgctcttccgatctattaagagatgtgtataagagacag

SEQ ID NO: 66 - DNA, 99 bp, Synthetic construct
  source 1..99: mol_type = other DNA
  misc_feature 1: note = 5' phosphorylation
  modified_base 41: mod_base = OTHER; note = deoxyuridine
caatcgagatcggaagagcacacgtctgaactccagtcactacactctttccctacacgacgctcttccgatctcaatcgagatgtgtataagagacag

SEQ ID NO: 67 - DNA, 99 bp, Synthetic construct
  source 1..99: mol_type = other DNA
  misc_feature 1: note = 5' phosphorylation
  modified_base 41: mod_base = OTHER; note = deoxyuridine
caccacagatcggaagagcacacgtctgaactccagtcactacactctttccctacacgacgctcttccgatctcaccacagatgtgtataagagacag

SEQ ID NO: 68 - DNA, 99 bp, Synthetic construct
  source 1..99: mol_type = other DNA
  misc_feature 1: note = 5' phosphorylation
  modified_base 41: mod_base = OTHER; note = deoxyuridine
cagcctagatcggaagagcacacgtctgaactccagtcactacactctttccctacacgacgctcttccgatctcagcctagatgtgtataagagacag

SEQ ID NO: 69 - DNA, 99 bp, Synthetic construct
  source 1..99: mol_type = other DNA
  misc_feature 1: note = 5' phosphorylation
  modified_base 41: mod_base = OTHER; note = deoxyuridine
cataggagatcggaagagcacacgtctgaactccagtcactacactctttccctacacgacgctcttccgatctcataggagatgtgtataagagacag

SEQ ID NO: 70 - DNA, 99 bp, Synthetic construct
  source 1..99: mol_type = other DNA
  misc_feature 1: note = 5' phosphorylation
  modified_base 41: mod_base = OTHER; note = deoxyuridine
catgccagatcggaagagcacacgtctgaactccagtcactacactctttccctacacgacgctcttccgatctcatgccagatgtgtataagagacag

SEQ ID NO: 71 - DNA, 99 bp, Synthetic construct
  source 1..99: mol_type = other DNA
  misc_feature 1: note = 5' phosphorylation
  modified_base 41: mod_base = OTHER; note = deoxyuridine
ccagacagatcggaagagcacacgtctgaactccagtcactacactctttccctacacgacgctcttccgatctccagacagatgtgtataagagacag

SEQ ID NO: 72 - DNA, 99 bp, Synthetic construct
  source 1..99: mol_type = other DNA
  misc_feature 1: note = 5' phosphorylation
  modified_base 41: mod_base = OTHER; note = deoxyuridine
ccatgtagatcggaagagcacacgtctgaactccagtcactacactctttccctacacgacgctcttccgatctccatgtagatgtgtataagagacag

SEQ ID NO: 73 - DNA, 99 bp, Synthetic construct
  source 1..99: mol_type = other DNA
  misc_feature 1: note = 5' phosphorylation
  modified_base 41: mod_base = OTHER; note = deoxyuridine
ccgaagagatcggaagagcacacgtctgaactccagtcactacactctttccctacacgacgctcttccgatctccgaagagatgtgtataagagacag

SEQ ID NO: 74 - DNA, 99 bp, Synthetic construct
  source 1..99: mol_type = other DNA
  misc_feature 1: note = 5' phosphorylation
  modified_base 41: mod_base = OTHER; note = deoxyuridine
ccgttcagatcggaagagcacacgtctgaactccagtcactacactctttccctacacgacgctcttccgatctccgttcagatgtgtataagagacag

SEQ ID NO: 75 - DNA, 99 bp, Synthetic construct
  source 1..99: mol_type = other DNA
  misc_feature 1: note = 5' phosphorylation
  modified_base 41: mod_base = OTHER; note = deoxyuridine
cgacagagatcggaagagcacacgtctgaactccagtcactacactctttccctacacgacgctcttccgatctcgacagagatgtgtataagagacag

SEQ ID NO: 76 - DNA, 99 bp, Synthetic construct
  source 1..99: mol_type = other DNA
  misc_feature 1: note = 5' phosphorylation
  modified_base 41: mod_base = OTHER; note = deoxyuridine
cgcgttagatcggaagagcacacgtctgaactccagtcactacactctttccctacacgacgctcttccgatctcgcgttagatgtgtataagagacag

SEQ ID NO: 77 - DNA, 99 bp, Synthetic construct
  source 1..99: mol_type = other DNA
  misc_feature 1: note = 5' phosphorylation
  modified_base 41: mod_base = OTHER; note = deoxyuridine
cggtgaagatcggaagagcacacgtctgaactccagtcactacactctttccctacacgacgctcttccgatctcggtgaagatgtgtataagagacag

SEQ ID NO: 78 - DNA, 99 bp, Synthetic construct
  source 1..99: mol_type = other DNA
  misc_feature 1: note = 5' phosphorylation
  modified_base 41: mod_base = OTHER; note = deoxyuridine
cgtataagatcggaagagcacacgtctgaactccagtcactacactctttccctacacgacgctcttccgatctcgtataagatgtgtataagagacag

SEQ ID NO: 79 - DNA, 99 bp, Synthetic construct
  source 1..99: mol_type = other DNA
  misc_feature 1: note = 5' phosphorylation
  modified_base 41: mod_base = OTHER; note = deoxyuridine
ctaattagatcggaagagcacacgtctgaactccagtcactacactctttccctacacgacgctcttccgatctctaattagatgtgtataagagacag

SEQ ID NO: 80 - DNA, 99 bp, Synthetic construct
  source 1..99: mol_type = other DNA
  misc_feature 1: note = 5' phosphorylation
  modified_base 41: mod_base = OTHER; note = deoxyuridine
ctcagcagatcggaagagcacacgtctgaactccagtcactacactctttccctacacgacgctcttccgatctctcagcagatgtgtataagagacag

SEQ ID NO: 81 - DNA, 99 bp, Synthetic construct
  source 1..99: mol_type = other DNA
  misc_feature 1: note = 5' phosphorylation
  modified_base 41: mod_base = OTHER; note = deoxyuridine
ctctcaagatcggaagagcacacgtctgaactccagtcactacactctttccctacacgacgctcttccgatctctctcaagatgtgtataagagacag

SEQ ID NO: 82 - DNA, 99 bp, Synthetic construct
  source 1..99: mol_type = other DNA
  misc_feature 1: note = 5' phosphorylation
  modified_base 41: mod_base = OTHER; note = deoxyuridine
cttcgtagatcggaagagcacacgtctgaactccagtcactacactctttccctacacgacgctcttccgatctcttcgtagatgtgtataagagacag

SEQ ID NO: 83 - DNA, 99 bp, Synthetic construct
  source 1..99: mol_type = other DNA
  misc_feature 1: note = 5' phosphorylation
  modified_base 41: mod_base = OTHER; note = deoxyuridine
cttgaaagatcggaagagcacacgtctgaactccagtcactacactctttccctacacgacgctcttccgatctcttgaaagatgtgtataagagacag

SEQ ID NO: 84 - DNA, 99 bp, Synthetic construct
  source 1..99: mol_type = other DNA
  misc_feature 1: note = 5' phosphorylation
  modified_base 41: mod_base = OTHER; note = deoxyuridine
gaagtaagatcggaagagcacacgtctgaactccagtcactacactctttccctacacgacgctcttccgatctgaagtaagatgtgtataagagacag

SEQ ID NO: 85 - DNA, 99 bp, Synthetic construct
  source 1..99: mol_type = other DNA
  misc_feature 1: note = 5' phosphorylation
  modified_base 41: mod_base = OTHER; note = deoxyuridine
gacagaagatcggaagagcacacgtctgaactccagtcactacactctttccctacacgacgctcttccgatctgacagaagatgtgtataagagacag

SEQ ID NO: 86 - DNA, 99 bp, Synthetic construct
  source 1..99: mol_type = other DNA
  misc_feature 1: note = 5' phosphorylation
  modified_base 41: mod_base = OTHER; note = deoxyuridine
gagaacagatcggaagagcacacgtctgaactccagtcactacactctttccctacacgacgctcttccgatctgagaacagatgtgtataagagacag

SEQ ID NO: 87 - DNA, 99 bp, Synthetic construct
  source 1..99: mol_type = other DNA
  misc_feature 1: note = 5' phosphorylation
  modified_base 41: mod_base = OTHER; note = deoxyuridine
gatcttagatcggaagagcacacgtctgaactccagtcactacactctttccctacacgacgctcttccgatctgatcttagatgtgtataagagacag

SEQ ID NO: 88 - DNA, 99 bp, Synthetic construct
  source 1..99: mol_type = other DNA
  misc_feature 1: note = 5' phosphorylation
  modified_base 41: mod_base = OTHER; note = deoxyuridine
gcaacgagatcggaagagcacacgtctgaactccagtcactacactctttccctacacgacgctcttccgatctgcaacgagatgtgtataagagacag

SEQ ID NO: 89 - DNA, 99 bp, Synthetic construct
  source 1..99: mol_type = other DNA
  misc_feature 1: note = 5' phosphorylation
  modified_base 41: mod_base = OTHER; note = deoxyuridine
gcgagtagatcggaagagcacacgtctgaactccagtcactacactctttccctacacgacgctcttccgatctgcgagtagatgtgtataagagacag

SEQ ID NO: 90 - DNA, 99 bp, Synthetic construct
  source 1..99: mol_type = other DNA
  misc_feature 1: note = 5' phosphorylation
  modified_base 41: mod_base = OTHER; note = deoxyuridine
gcggaaagatcggaagagcacacgtctgaactccagtcactacactctttccctacacgacgctcttccgatctgcggaaagatgtgtataagagacag

SEQ ID NO: 91 - DNA, 99 bp, Synthetic construct
  source 1..99: mol_type = other DNA
  misc_feature 1: note = 5' phosphorylation
  modified_base 41: mod_base = OTHER; note = deoxyuridine
gctccaagatcggaagagcacacgtctgaactccagtcactacactctttccctacacgacgctcttccgatctgctccaagatgtgtataagagacag

SEQ ID NO: 92 - DNA, 99 bp, Synthetic construct
  source 1..99: mol_type = other DNA
  misc_feature 1: note = 5' phosphorylation
  modified_base 41: mod_base = OTHER; note = deoxyuridine
ggatcaagatcggaagagcacacgtctgaactccagtcactacactctttccctacacgacgctcttccgatctggatcaagatgtgtataagagacag

SEQ ID NO: 93 - DNA, 99 bp, Synthetic construct
  source 1..99: mol_type = other DNA
  misc_feature 1: note = 5' phosphorylation
  modified_base 41: mod_base = OTHER; note = deoxyuridine
ggccaaagatcggaagagcacacgtctgaactccagtcactacactctttccctacacgacgctcttccgatctggccaaagatgtgtataagagacag

SEQ ID NO: 94 - DNA, 99 bp, Synthetic construct
  source 1..99: mol_type = other DNA
  misc_feature 1: note = 5' phosphorylation
  modified_base 41: mod_base = OTHER; note = deoxyuridine
ggcttcagatcggaagagcacacgtctgaactccagtcactacactctttccctacacgacgctcttccgatctggcttcagatgtgtataagagacag

SEQ ID NO: 95 - DNA, 99 bp, Synthetic construct
  source 1..99: mol_type = other DNA
  misc_feature 1: note = 5' phosphorylation
  modified_base 41: mod_base = OTHER; note = deoxyuridine
ggttggagatcggaagagcacacgtctgaactccagtcactacactctttccctacacgacgctcttccgatctggttggagatgtgtataagagacag

SEQ ID NO: 96 - DNA, 99 bp, Synthetic construct
  source 1..99: mol_type = other DNA
  misc_feature 1: note = 5' phosphorylation
  modified_base 41: mod_base = OTHER; note = deoxyuridine
gtatatagatcggaagagcacacgtctgaactccagtcactacactctttccctacacgacgctcttccgatctgtatatagatgtgtataagagacag

SEQ ID NO: 97 - DNA, 99 bp, Synthetic construct
  source 1..99: mol_type = other DNA
  misc_feature 1: note = 5' phosphorylation
  modified_base 41: mod_base = OTHER; note = deoxyuridine
gttaccagatcggaagagcacacgtctgaactccagtcactacactctttccctacacgacgctcttccgatctgttaccagatgtgtataagagacag

SEQ ID NO: 98 - DNA, 99 bp, Synthetic construct
  source 1..99: mol_type = other DNA
  misc_feature 1: note = 5' phosphorylation
  modified_base 41: mod_base = OTHER; note = deoxyuridine
gttgtgagatcggaagagcacacgtctgaactccagtcactacactctttccctacacgacgctcttccgatctgttgtgagatgtgtataagagacag

SEQ ID NO: 99 - DNA, 99 bp, Synthetic construct
  source 1..99: mol_type = other DNA
  misc_feature 1: note = 5' phosphorylation
  modified_base 41: mod_base = OTHER; note = deoxyuridine
taaccaagatcggaagagcacacgtctgaactccagtcactacactctttccctacacgacgctcttccgatcttaaccaagatgtgtataagagacag

SEQ ID NO: 100 - DNA, 99 bp, Synthetic construct
  source 1..99: mol_type = other DNA
  misc_feature 1: note = 5' phosphorylation
  modified_base 41: mod_base = OTHER; note = deoxyuridine
taagatagatcggaagagcacacgtctgaactccagtcactacactctttccctacacgacgctcttccgatcttaagatagatgtgtataagagacag

SEQ ID NO: 101 - DNA, 99 bp, Synthetic construct
  source 1..99: mol_type = other DNA
  misc_feature 1: note = 5' phosphorylation
  modified_base 41: mod_base = OTHER; note = deoxyuridine
tacatcagatcggaagagcacacgtctgaactccagtcactacactctttccctacacgacgctcttccgatcttacatcagatgtgtataagagacag

SEQ ID NO: 102 - DNA, 99 bp, Synthetic construct
  source 1..99: mol_type = other DNA
  misc_feature 1: note = 5' phosphorylation
  modified_base 41: mod_base = OTHER; note = deoxyuridine
taggtgagatcggaagagcacacgtctgaactccagtcactacactctttccctacacgacgctcttccgatcttaggtgagatgtgtataagagacag

SEQ ID NO: 103 - DNA, 99 bp, Synthetic construct
  source 1..99: mol_type = other DNA
  misc_feature 1: note = 5' phosphorylation
  modified_base 41: mod_base = OTHER; note = deoxyuridine
tatcagagatcggaagagcacacgtctgaactccagtcactacactctttccctacacgacgctcttccgatcttatcagagatgtgtataagagacag

SEQ ID NO: 104 - DNA, 99 bp, Synthetic construct
  source 1..99: mol_type = other DNA
  misc_feature 1: note = 5' phosphorylation
  modified_base 41: mod_base = OTHER; note = deoxyuridine
tattgcagatcggaagagcacacgtctgaactccagtcactacactctttccctacacgacgctcttccgatcttattgcagatgtgtataagagacag

SEQ ID NO: 105 - DNA, 99 bp, Synthetic construct
  source 1..99: mol_type = other DNA
  misc_feature 1: note = 5' phosphorylation
  modified_base 41: mod_base = OTHER; note = deoxyuridine
tcacgcagatcggaagagcacacgtctgaactccagtcactacactctttccctacacgacgctcttccgatcttcacgcagatgtgtataagagacag

SEQ ID NO: 106 - DNA, 99 bp, Synthetic construct
  source 1..99: mol_type = other DNA
  misc_feature 1: note = 5' phosphorylation
  modified_base 41: mod_base = OTHER; note = deoxyuridine
tccttaagatcggaagagcacacgtctgaactccagtcactacactctttccctacacgacgctcttccgatcttccttaagatgtgtataagagacag

SEQ ID NO: 107 - DNA, 99 bp, Synthetic construct
  source 1..99: mol_type = other DNA
  misc_feature 1: note = 5' phosphorylation
  modified_base 41: mod_base = OTHER; note = deoxyuridine
tcggctagatcggaagagcacacgtctgaactccagtcactacactctttccctacacgacgctcttccgatcttcggctagatgtgtataagagacag

SEQ ID NO: 108 - DNA, 99 bp, Synthetic construct
  source 1..99: mol_type = other DNA
  misc_feature 1: note = 5' phosphorylation
  modified_base 41: mod_base = OTHER; note = deoxyuridine
tgaagtagatcggaagagcacacgtctgaactccagtcactacactctttccctacacgacgctcttccgatcttgaagtagatgtgtataagagacag

SEQ ID NO: 109 - DNA, 99 bp, Synthetic construct
  source 1..99: mol_type = other DNA
  misc_feature 1: note = 5' phosphorylation
  modified_base 41: mod_base = OTHER; note = deoxyuridine
tgcgagagatcggaagagcacacgtctgaactccagtcactacactctttccctacacgacgctcttccgatcttgcgagagatgtgtataagagacag

SEQ ID NO: 110 - DNA, 99 bp, Synthetic construct
  source 1..99: mol_type = other DNA
  misc_feature 1: note = 5' phosphorylation
  modified_base 41: mod_base = OTHER; note = deoxyuridine
tggctcagatcggaagagcacacgtctgaactccagtcactacactctttccctacacgacgctcttccgatcttggctcagatgtgtataagagacag

SEQ ID NO: 111 - DNA, 99 bp, Synthetic construct
  source 1..99: mol_type = other DNA
  misc_feature 1: note = 5' phosphorylation
  modified_base 41: mod_base = OTHER; note = deoxyuridine
tgtaacagatcggaagagcacacgtctgaactccagtcactacactctttccctacacgacgctcttccgatcttgtaacagatgtgtataagagacag

SEQ ID NO: 112 - DNA, 99 bp, Synthetic construct
  source 1..99: mol_type = other DNA
  misc_feature 1: note = 5' phosphorylation
  modified_base 41: mod_base = OTHER; note = deoxyuridine
tgttctagatcggaagagcacacgtctgaactccagtcactacactctttccctacacgacgctcttccgatcttgttctagatgtgtataagagacag

SEQ ID NO: 113 - DNA, 99 bp, Synthetic construct
  source 1..99: mol_type = other DNA
  misc_feature 1: note = 5' phosphorylation
  modified_base 41: mod_base = OTHER; note = deoxyuridine
ttattgagatcggaagagcacacgtctgaactccagtcactacactctttccctacacgacgctcttccgatctttattgagatgtgtataagagacag

SEQ ID NO: 114 - DNA, 99 bp, Synthetic construct
  source 1..99: mol_type = other DNA
  misc_feature 1: note = 5' phosphorylation
  modified_base 41: mod_base = OTHER; note = deoxyuridine
ttcggtagatcggaagagcacacgtctgaactccagtcactacactctttccctacacgacgctcttccgatctttcggtagatgtgtataagagacag

SEQ ID NO: 115 - DNA, 99 bp, Synthetic construct
  source 1..99: mol_type = other DNA
  misc_feature 1: note = 5' phosphorylation
  modified_base 41: mod_base = OTHER; note = deoxyuridine
ttgagaagatcggaagagcacacgtctgaactccagtcactacactctttccctacacgacgctcttccgatctttgagaagatgtgtataagagacag

SEQ ID NO: 116 - DNA, 99 bp, Synthetic construct
  source 1..99: mol_type = other DNA
  misc_feature 1: note = 5' phosphorylation
  modified_base 41: mod_base = OTHER; note = deoxyuridine
ttgccgagatcggaagagcacacgtctgaactccagtcactacactctttccctacacgacgctcttccgatctttgccgagatgtgtataagagacag

SEQ ID NO: 117 - DNA, 99 bp, Synthetic construct
  source 1..99: mol_type = other DNA
  misc_feature 1: note = 5' phosphorylation
  modified_base 41: mod_base = OTHER; note = deoxyuridine
aagtccagatcggaagagcacacgtctgaactccagtcactacactctttccctacacgacgctcttccgatctaagtccagatgtgtataagagacag

SEQ ID NO: 118 - DNA, 99 bp, Synthetic construct
  source 1..99: mol_type = other DNA
  misc_feature 1: note = 5' phosphorylation
  modified_base 41: mod_base = OTHER; note = deoxyuridine
acacacagatcggaagagcacacgtctgaactccagtcactacactctttccctacacgacgctcttccgatctacacacagatgtgtataagagacag

SEQ ID NO: 119 - DNA, 99 bp, Synthetic construct
  source 1..99: mol_type = other DNA
  misc_feature 1: note = 5' phosphorylation
  modified_base 41: mod_base = OTHER; note = deoxyuridine
acgcagagatcggaagagcacacgtctgaactccagtcactacactctttccctacacgacgctcttccgatctacgcagagatgtgtataagagacag

SEQ ID NO: 120 - DNA, 99 bp, Synthetic construct
  source 1..99: mol_type = other DNA
  misc_feature 1: note = 5' phosphorylation
  modified_base 41: mod_base = OTHER; note = deoxyuridine
acgtatagatcggaagagcacacgtctgaactccagtcactacactctttccctacacgacgctcttccgatctacgtatagatgtgtataagagacag

SEQ ID NO: 121 - DNA, 99 bp, Synthetic construct
  source 1..99: mol_type = other DNA
  misc_feature 1: note = 5' phosphorylation
  modified_base 41: mod_base = OTHER; note = deoxyuridine
actaacagatcggaagagcacacgtctgaactccagtcactacactctttccctacacgacgctcttccgatctactaacagatgtgtataagagacag

SEQ ID NO: 122 - DNA, 99 bp, Synthetic construct
  source 1..99: mol_type = other DNA
  misc_feature 1: note = 5' phosphorylation
  modified_base 41: mod_base = OTHER; note = deoxyuridine
agcattagatcggaagagcacacgtctgaactccagtcactacactctttccctacacgacgctcttccgatctagcattagatgtgtataagagacag

SEQ ID NO: 123 - DNA, 99 bp, Synthetic construct
  source 1..99: mol_type = other DNA
  misc_feature 1: note = 5' phosphorylation
  modified_base 41: mod_base = OTHER; note = deoxyuridine
agcctcagatcggaagagcacacgtctgaactccagtcactacactctttccctacacgacgctcttccgatctagcctcagatgtgtataagagacag

SEQ ID NO: 124 - DNA, 99 bp, Synthetic construct
  source 1..99: mol_type = other DNA
  misc_feature 1: note = 5' phosphorylation
  modified_base 41: mod_base = OTHER; note = deoxyuridine
aggagtagatcggaagagcacacgtctgaactccagtcactacactctttccctacacgacgctcttccgatctaggagtagatgtgtataagagacag

SEQ ID NO: 125 - DNA, 99 bp, Synthetic construct
  source 1..99: mol_type = other DNA
  misc_feature 1: note = 5' phosphorylation
  modified_base 41: mod_base = OTHER; note = deoxyuridine
atgacaagatcggaagagcacacgtctgaactccagtcactacactctttccctacacgacgctcttccgatctatgacaagatgtgtataagagacag

SEQ ID NO: 126 - DNA, 99 bp, Synthetic construct
  source 1..99: mol_type = other DNA
  misc_feature 1: note = 5' phosphorylation
  modified_base 41: mod_base = OTHER; note = deoxyuridine
attccgagatcggaagagcacacgtctgaactccagtcactacactctttccctacacgacgctcttccgatctattccgagatgtgtataagagacag

SEQ ID NO: 127 - DNA, 99 bp, Synthetic construct
  source 1..99: mol_type = other DNA
  misc_feature 1: note = 5' phosphorylation
  modified_base 41: mod_base = OTHER; note = deoxyuridine
cactgaagatcggaagagcacacgtctgaactccagtcactacactctttccctacacgacgctcttccgatctcactgaagatgtgtataagagacag

SEQ ID NO: 128 - DNA, 99 bp, Synthetic construct
  source 1..99: mol_type = other DNA
  misc_feature 1: note = 5' phosphorylation
  modified_base 41: mod_base = OTHER; note = deoxyuridine
ccaaggagatcggaagagcacacgtctgaactccagtcactacactctttccctacacgacgctcttccgatctccaaggagatgtgtataagagacag

SEQ ID NO: 129 - DNA, 99 bp, Synthetic construct
  source 1..99: mol_type = other DNA
  misc_feature 1: note = 5' phosphorylation
  modified_base 41: mod_base = OTHER; note = deoxyuridine
cgatgcagatcggaagagcacacgtctgaactccagtcactacactctttccctacacgacgctcttccgatctcgatgcagatgtgtataagagacag

SEQ ID NO: 130 - DNA, 99 bp, Synthetic construct
  source 1..99: mol_type = other DNA
  misc_feature 1: note = 5' phosphorylation
  modified_base 41: mod_base = OTHER; note = deoxyuridine
cgtaagagatcggaagagcacacgtctgaactccagtcactacactctttccctacacgacgctcttccgatctcgtaagagatgtgtataagagacag

SEQ ID NO: 131 - DNA, 99 bp, Synthetic construct
  source 1..99: mol_type = other DNA
  misc_feature 1: note = 5' phosphorylation
  modified_base 41: mod_base = OTHER; note = deoxyuridine
ctatccagatcggaagagcacacgtctgaactccagtcactacactctttccctacacgacgctcttccgatctctatccagatgtgtataagagacag

SEQ ID NO: 132 - DNA, 99 bp, Synthetic construct
  source 1..99: mol_type = other DNA
  misc_feature 1: note = 5' phosphorylation
  modified_base 41: mod_base = OTHER; note = deoxyuridine
ctcaagagatcggaagagcacacgtctgaactccagtcactacactctttccctacacgacgctcttccgatctctcaagagatgtgtataagagacag

SEQ ID NO: 133 - DNA, 99 bp, Synthetic construct
  source 1..99: mol_type = other DNA
  misc_feature 1: note = 5' phosphorylation
  modified_base 41: mod_base = OTHER; note = deoxyuridine
gaatggagatcggaagagcacacgtctgaactccagtcactacactctttccctacacgacgctcttccgatctgaatggagatgtgtataagagacag

SEQ ID NO: 134 - DNA, 99 bp, Synthetic construct
  source 1..99: mol_type = other DNA
  misc_feature 1: note = 5' phosphorylation
  modified_base 41: mod_base = OTHER; note = deoxyuridine
gacattagatcggaagagcacacgtctgaactccagtcactacactctttccctacacgacgctcttccgatctgacattagatgtgtataagagacag

SEQ ID NO: 135 - DNA, 99 bp, Synthetic construct
  source 1..99: mol_type = other DNA
  misc_feature 1: note = 5' phosphorylation
  modified_base 41: mod_base = OTHER; note = deoxyuridine
gacctcagatcggaagagcacacgtctgaactccagtcactacactctttccctacacgacgctcttccgatctgacctcagatgtgtataagagacag

SEQ ID NO: 136 - DNA, 99 bp, Synthetic construct
  source 1..99: mol_type = other DNA
  misc_feature 1: note = 5' phosphorylation
  modified_base 41: mod_base = OTHER; note = deoxyuridine
gatccgagatcggaagagcacacgtctgaactccagtcactacactctttccctacacgacgctcttccgatctgatccgagatgtgtataagagacag

SEQ ID NO: 137 - DNA, 99 bp, Synthetic construct
  source 1..99: mol_type = other DNA
  misc_feature 1: note = 5' phosphorylation
  modified_base 41: mod_base = OTHER; note = deoxyuridine
gcattgagatcggaagagcacacgtctgaactccagtcactacactctttccctacacgacgctcttccgatctgcattgagatgtgtataagagacag

SEQ ID NO: 138 - DNA, 99 bp, Synthetic construct
  source 1..99: mol_type = other DNA
  misc_feature 1: note = 5' phosphorylation
  modified_base 41: mod_base = OTHER; note = deoxyuridine
gctaagagatcggaagagcacacgtctgaactccagtcactacactctttccctacacgacgctcttccgatctgctaagagatgtgtataagagacag

SEQ ID NO: 139 - DNA, 99 bp, Synthetic construct
  source 1..99: mol_type = other DNA
  misc_feature 1: note = 5' phosphorylation
  modified_base 41: mod_base = OTHER; note = deoxyuridine
gctactagatcggaagagcacacgtctgaactccagtcactacactctttccctacacgacgctcttccgatctgctactagatgtgtataagagacag

SEQ ID NO: 140 - DNA, 99 bp, Synthetic construct
  source 1..99: mol_type = other DNA
  misc_feature 1: note = 5' phosphorylation
  modified_base 41: mod_base = OTHER; note = deoxyuridine
gtcatgagatcggaagagcacacgtctgaactccagtcactacactctttccctacacgacgctcttccgatctgtcatgagatgtgtataagagacag

SEQ ID NO: 141 - DNA, 99 bp, Synthetic construct
  source 1..99: mol_type = other DNA
  misc_feature 1: note = 5' phosphorylation
  modified_base 41: mod_base = OTHER; note = deoxyuridine
gtgaggagatcggaagagcacacgtctgaactccagtcactacactctttccctacacgacgctcttccgatctgtgaggagatgtgtataagagacag

SEQ ID NO: 142 - DNA, 99 bp, Synthetic construct
  source 1..99: mol_type = other DNA
  misc_feature 1: note = 5' phosphorylation
  modified_base 41: mod_base = OTHER; note = deoxyuridine
gtgcgaagatcggaagagcacacgtctgaactccagtcactacactctttccctacacgacgctcttccgatctgtgcgaagatgtgtataagagacag

SEQ ID NO: 143 - DNA, 99 bp, Synthetic construct
  source 1..99: mol_type = other DNA
  misc_feature 1: note = 5' phosphorylation
  modified_base 41: mod_base = OTHER; note = deoxyuridine
taagtcagatcggaagagcacacgtctgaactccagtcactacactctttccctacacgacgctcttccgatcttaagtcagatgtgtataagagacag

SEQ ID NO: 144 - DNA, 47 bp, Synthetic construct
  source 1..47: mol_type = other DNA
caagcagaagacggcatacgagattcgatccagtctcgtgggctcgg

SEQ ID NO: 145 - DNA, 99 bp, Synthetic construct
  source 1..99: mol_type = other DNA
  misc_feature 1: note = 5' phosphorylation
  modified_base 41: mod_base = OTHER; note = deoxyuridine
nnnnnnagatcggaagagcacacgtctgaactccagtcactacactctttccctacacgacgctcttccgatctnnnnnnagatgtgtataagagacag

SEQ ID NO: 146 - DNA, 12 bp, Synthetic construct
  source 1..12: mol_type = other DNA
ctcagcgacctc

SEQ ID NO: 147 - DNA, 12 bp, Synthetic construct
  source 1..12: mol_type = other DNA
gacctcctcagc